(19) United States
(12) Patent Application Publication
(10) Pub. No.: US 2010/0068197 A1
(43) Pub. Date: Mar. 18, 2010

(54) PHARMACEUTICAL COMPOUNDS AS INHIBITORS OF CELL PROLIFERATION AND THE USE THEREOF

(75) Inventors: MARK B. ANDERSON, Salt Lake City, UT (US); J. Adam Willardsen, Salt Lake City, UT (US); Warren S. Weiner, Salt Lake City, UT (US); Kazuyuki Suzuki, Salt Lake City, UT (US); Robert J. Halter, Salt Lake City, UT (US); In Chul Kim, Salt Lake City, UT (US); Matthew Reeder, Salt Lake City, UT (US); Ashantai Yungai, Salt Lake City, UT (US)

Correspondence Address: Myriad PHARMACEUTICALS, Inc.;c/o CPA Global, P.O. Box 52050, Minneapolis, MN, 55402, US

(73) Assignee: Myriad Pharmaceuticals, Inc., Salt Lake City, UT (US)

(21) Appl. No.: 12/500,449

(22) Filed: Jul. 9, 2009

Related U.S. Application Data
(60) Provisional application No. 61/079,890, filed on Jul. 11, 2008.

(51) Int. Cl. A61K 31/517; C07D 239/95; C07D 403/12; C07D 487/04; A61K 31/5025; A61P 35/00; A61K 39/395; C12N 5/00

(52) U.S. Cl. 424/130.1; 544/286; 544/291; 544/236; 544/284; 514/266.3; 514/266.4; 514/252.17; 514/248; 514/266.2; 435/375

(57) ABSTRACT

Disclosed are compounds of Formula I effective as cytotoxic agents. The compounds of this invention are useful in the treatment of a variety of clinical conditions in which uncontrolled growth and spread of abnormal cells occurs.

### CROSS-REFERENCE TO RELATED APPLICATIONS

This application claims the benefit under 35 U.S.C. §119(e) of U.S. provisional application Ser. No. 61/079,890, filed Jul. 11, 2008, the contents of which are hereby incorporated by reference in their entirety.

### FIELD OF THE INVENTION

This invention is in the field of medicinal chemistry. In particular, the invention relates to compounds that are cytotoxic agents. The invention also relates to the use of these compounds as therapeutically effective anti-cancer agents.

### BACKGROUND OF THE INVENTION

Cancer is a common cause of death in the world; about 10 million new cases occur each year, and cancer is responsible for 12% of deaths worldwide, making cancer the third leading cause of death. World Health Organization, National Cancer Control Programmes Policies and Managerial Guidelines (2d ed. 2002)

Despite advances in the field of cancer treatment, the leading therapies to date include surgery, radiation, and chemotherapy. Chemotherapeutic approaches are said to fight cancers that are metastasized or that are particularly aggressive. Most of the cancer chemotherapy agents currently in clinical use are cytotoxins. Cytotoxic agents work by damaging or killing cells that exhibit rapid growth. Ideal cytotoxic agents would have specificity for cancer and tumor cells, while not affecting normal cells. Unfortunately, none have been found and instead agents that target especially rapidly dividing cells (both tumor and normal) have been used.

Accordingly, discovery of new and effective treatments for cancer is a high priority for health care researchers. Materials that are cytotoxic to cancer cells while exerting only mild effects on normal cells are highly desirable. For this reason, there remains a definite need in the art for new effective chemotherapeutic agents.

### BRIEF SUMMARY OF THE INVENTION

The present invention is related to the discovery that compounds of Formula I below, are cytotoxic agents. Thus, they are useful in treating or delaying the onset of diseases and disorders that are responsive to cytotoxic agents.

Accordingly, one aspect of the present invention is directed to the use of compounds of the present invention in treating or ameliorating neoplasm and cancer, by administering the compounds to cells in vitro or in vivo in warm-blooded animals, particularly mammals.

Many of the compounds as represented by Formula I below are novel compounds. Therefore, another aspect of the present invention is to provide novel compounds.

Yet another aspect of the present invention is to provide a pharmaceutical composition useful for treating disorders responsive to cytotoxic agents, containing an effective amount of a compound of the present invention, preferably in admixture with one or more pharmaceutically acceptable carriers or diluents.

In yet another aspect of the present invention, methods are provided for the preparation of the novel compounds of the present invention.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

It has been discovered that compounds of the present invention are potent and highly efficacious cytotoxic agents. Therefore, the compounds are useful for treating diseases and disorders responsive to cytotoxic agents.

The above various methods of the present invention can be practiced by or comprise treating cells in vitro or a warm-blooded animal, particularly mammal, more particularly a human with an effective amount of a compound according to the present invention. As used herein, the phrase “treating . . . with . . . a compound” means either administering the compound to cells or an animal, or administering to cells or an animal the compound or another agent to cause the presence or formation of the compound inside the cells or the animal. Preferably, the methods of the present invention comprise administering to cells in vitro or to a warm-blooded animal, particularly mammal, more particularly a human a pharmaceutical composition comprising an effective amount of a compound according to the present invention.

Specifically, the methods of the present invention comprise treating cells in vitro or a warm-blooded animal, particularly mammal, more particularly a human with an effective amount of a compound according to Formula I:

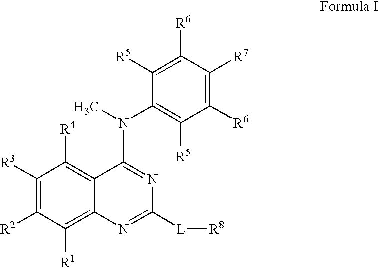

wherein,

R¹, R², R³, R⁴, R⁵, and R⁶ are each independently chosen from the group consisting of H, C₁₋₃alkyl, C₁₋₃alkoxy, and halo;

R⁷ is C₁₋₃alkoxy;

L is chosen from —OCH₂CH₂—, —NHCH₂CH₂—, —CH₂NHC(═O)—, —CH₂NHS(═O)₂—, or —CH₂OS(═O)₂—; each optionally substituted with one or more H or C₁₋₃alkyl;

with R⁸ chosen from H, OH, amino, 2,6-diamino-hexanoic acid, 2-amino-3-(1H-indol-3-yl)-propionic acid, 2,6-diamino-hexanoic acid, 2-amino-4-methylsulfanyl-butyric acid, 2-amino-succinamic acid, 2-benzyloxycarbonylamino-3-(1H-indol-3-yl)-propionic acid, 2-(2-amino-ethoxy)-ethanol, 2-(2-ethoxy-ethoxy)-ethanol, 2-(2-methoxy-ethoxy)-ethanol, 2-amino-acetamide, 2-methoxy-ethanol, 2-piperazin-1-yl-ethanol, 8-methyl-1,3-dioxo-2,3,5,8-tetrahydro-1H-[1,2,4]triazolo[1,2-a]pyridazine-5-carboxylic acid ethyl ester, acetamide, amino-acetic acid, carbamic acid methyl ester, carbamic acid tert-butyl ester, carbamoylmethyl-carbamic acid tert-butyl ester, carbonic acid mono-[2-(2-methoxy-ethoxy)-ethyl]ester, ethyl-carbamic acid tert-butyl ester, isobutyric acid 1-hydroxy-ethyl ester, methoxy-benzene, methyl-urea, N¹-(2-Amino-ethyl)-ethane-1,2-diamine, phenol, phenyl-methanol, phosphoric acid di-tent-butyl ester, phosphoric acid mono-tent-butyl ester, (2-Carbamoyl-ethyl)-carbamic acid tert-butyl ester, 3-amino-propionamide, acetic acid 3-acetoxy-2-hydroxy-propyl ester, or urea;

each optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle;

or L is a direct bond; with R⁸ chosen from 3-({4-[(4-methoxy-phenyl)-methyl-amino]-quinazolin-2-ylmethyl}-methyl-amino)-propan-1-ol, N⁴-(4-methoxy-phenyl)-N⁴-methyl-N²-(3-methylamino-propyl)-quinazoline-2,4-diamine, 2-amino-1-phenyl-propan-1-ol, 2-amino-2-phenyl-ethanol, 2-amino-3-(4-hydroxy-phenyl)-propionic acid methyl ester, 2-amino-3-phenyl-propan-1-ol, 2-amino-propionic acid, 2-amino-propionic acid tert-butyl ester, 2-amino-3-methyl-butyric acid ethyl ester, N-(2-acetylamino-ethyl)-acetamide, 1-amino-3-{4-[(4-methoxy-phenyl)-methyl-amino]-quinazolin-2-ylamino}-propan-2-ol, 1,3-diamino-propan-2-ol, or piperazine; optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle.

In some embodiments of Formula I, R¹, R², R⁴, and R⁵ are each independently H or C₁₋₃alkyl. In some embodiments R¹, R², R⁴ and R⁵ are H.

In some embodiments of Formula I, R³ is chosen from —OCH₃, —CH₃, and F. In specific embodiments, R³ is chosen from —CH₃ or —OCH₃. In one embodiment, R³ is F.

In some embodiments of Formula I, each R⁶ is independently chosen from H and C₁₋₃alkoxy. In some embodiments, R⁷ is —OCH₃.

In some embodiments of Formula I, L is chosen from —OCH₂CH₂—, —NHCH₂CH₂—, —CH₂NHC(═O)—, or —CH₂NHS(═O)₂—; with R⁸ chosen from H, (R)-2,6-diamino-hexanoic acid, (R)-2-amino-3-(1H-indol-3-yl)-propionic acid, (S)-2,6-diamino-hexanoic acid, (S)-2-amino-4-methylsulfanyl-butyric acid, (S)-2-amino-succinamic acid, (S)-2-benzyloxycarbonylamino-3-(1H-indol-3-yl)-propionic acid, 2-(2-amino-ethoxy)-ethanol, 2-(2-ethoxy-ethoxy)-ethanol, 2-(2-methoxy-ethoxy)-ethanol, 2-amino-acetamide, 2-methoxy-ethanol, 2-piperazin-1-yl-ethanol, 8-methyl-1,3-dioxo-2,3,5,8-tetrahydro-1H-[1,2,4]triazolo[1,2-a]pyridazine-5-carboxylic acid ethyl ester, acetamide, amino-acetic acid, carbamic acid methyl ester, carbamic acid tert-butyl ester, carbamoylmethyl-carbamic acid tert-butyl ester, carbonic acid mono-[2-(2-methoxy-ethoxy)-ethyl]ester, ethyl-carbamic acid tert-butyl ester, isobutyric acid 1-hydroxy-ethyl ester, methoxy-benzene, methyl-urea, phenol, phenyl-methanol, phosphoric acid di-tent-butyl ester, phosphoric acid mono-tent-butyl ester, (2-carbamoyl-ethyl)-carbamic acid tert-butyl ester, 3-amino-propionamide, acetic acid 3-acetoxy-2-hydroxy-propyl ester, or urea; optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆ alkyl, C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle;

or L is a direct bond; with R⁸ chosen from N⁴-(4-methoxy-phenyl)-N⁴-methyl-N²-(3-methylamino-propyl)-quinazoline-2,4-diamine, (1R,2S)-2-amino-1-phenyl-propan-1-ol, (S)-2-amino-2-phenyl-ethanol, (S)-2-amino-3-(4-hydroxy-phenyl)-propionic acid methyl ester, (S)-2-amino-3-phenyl-propan-1-ol, (S)-2-amino-propionic acid, (S)-2-amino-propionic acid tert-butyl ester, 2-amino-3-methyl-butyric acid ethyl ester, N-(2-acetylamino-ethyl)-acetamide, 1-amino-3-{4-[(4-methoxy-phenyl)-methyl-amino]-quinazolin-2-ylamino}-propan-2-ol, 1,3-diamino-propan-2-ol, or piperazine; optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle.

In some embodiments of Formula I, L is —NHCH₂CH₂—, and R⁸ is chosen from 2-(2-amino-ethoxy)-ethanol, N¹-(2-amino-ethyl)-ethane-1,2-diamine, 8-methyl-1,3-dioxo-2,3,5,8-tetrahydro-1H-[1,2,4]triazolo[1,2-a]pyridazine-5-carboxylic acid ethyl ester, carbonic acid mono-[2-(2-methoxy-ethoxy)-ethyl]ester, phosphoric acid di-tent-butyl ester, (S)-2,6-diamino-hexanoic acid, phosphoric acid mono-tent-butyl ester, amino-acetic acid, carbamic acid tert-butyl ester, (R)-2,6-diamino-hexanoic acid, carbamic acid benzyl ester, carbamic acid methyl ester, (S)-2-amino-4-methylsulfanyl-butyric acid, (S)-2-benzyloxycarbonylamino-3-(1H-indol-3-yl)-propionic acid, carbamoylmethyl-carbamic acid tert-butyl ester, (S)-2-amino-succinamic acid, (R)-2-amino-3-(1H-indol-3-yl)-propionic acid, methyl-urea, 2-amino-acetamide, acetamide, and urea.

In other embodiments, of Formula I, L is —OCH₂CH₂—, and R⁸ is chosen from 2-methoxy-ethanol, 2-(2-methoxy-ethoxy)-ethanol, 2-piperazin-1-yl-ethanol, and 2-(2-ethoxy-ethoxy)-ethanol. In another embodiment, L is —CH₂NHC(═O)—, and R⁸ is chosen from ethyl-carbamic acid tert-butyl ester, isobutyric acid 1-hydroxy-ethyl ester, phenyl-methanol, and phenol. In one embodiment, L is —CH₂NHS(═O)₂—, and R⁸ is methoxy-benzene.

Exemplary compounds of the present invention are compounds provided in Examples 1-55, and pharmaceutically acceptable salts or prodrugs thereof. Specific exemplary compounds include but are not limited to:
{2-[2-(2-methoxy-ethoxy)-ethoxy]-quinazolin-4-yl}-(4-methoxy-phenyl)-methyl-amine;
(2-{2-[2-(2-methoxy-ethoxy)-ethoxy]-ethoxy}-quinazolin-4-yl)-(4-methoxy-phenyl)-methyl-amine;
N²-{2-[2-(2-amino-ethoxy)-ethoxy]-ethyl}-N⁴-(4-methoxy-phenyl)-N⁴-methyl-quinazoline-2,4-diamine;
N²-{2-[2-(2-amino-ethylamino)-ethylamino]-ethyl}-N⁴-(4-methoxy-phenyl)-N⁴-methyl-quinazoline-2,4-diamine;
2-[4-(2-{4-[(4-methoxy-phenyl)-methyl-amino]-quinazolin-2-yloxy}-ethyl)-piperazin-1-yl]-ethanol;
2-(2-{4-[(4-methoxy-phenyl)-methyl-amino]-quinazolin-2-ylamino}-ethyl)-8-methyl-1,3-dioxo-2,3,5,8-tetrahydro-1-[1,2,4]triazolo[1,2-a]pyridazine-5-carboxylic acid ethyl ester;
(2-{2-[2-(2-ethoxy-ethoxy)-ethoxy]-ethoxy}-quinazolin-4-yl)-(4-methoxy-phenyl)-methyl-amine;
carbonic acid 2-(2-methoxy-ethoxy)-ethyl ester 2-{4-[(4-methoxy-phenyl)-methyl-amino]-quinazolin-2-ylamino}-ethyl ester;
(4-methoxy-phenyl)-methyl-(2-piperazin-1-yl-quinazolin-4-yl)-amine;
phosphoric acid di-tent-butyl ester 2-{4-[(4-methoxy-phenyl)-methyl-amino]-quinazolin-2-ylamino}-ethyl ester;
(S)-2,6-diamino-hexanoic acid 2-{4-[(4-methoxy-phenyl)-methyl-amino]-quinazolin-2-ylamino}-ethyl ester;
phosphoric acid tert-butyl ester 2-{4-[(4-methoxy-phenyl)-methyl-amino]-quinazolin-2-ylamino}-ethyl ester;
amino-acetic acid 2-{4-[(4-methoxy-phenyl)-methyl-amino]-quinazolin-2-ylamino}-ethyl ester;
(2-{4-[(3,4-dimethoxy-phenyl)-methyl-amino]-quinazolin-2-ylamino}-ethyl)-carbamic acid tert-butyl ester;
(R)-2,6-diamino-hexanoic acid 2-{4-[(4-methoxy-phenyl)-methyl-amino]-quinazolin-2-ylamino}-ethyl ester;
(2-{6-methoxy-4-[(4-methoxy-phenyl)-methyl-amino]-quinazolin-2-ylamino}-ethyl)-carbamic acid tert-butyl ester;
(2-{6-methoxy-4-[(4-methoxy-phenyl)-methyl-amino]-quinazolin-2-ylamino}-ethyl)-carbamic acid benzyl ester;
N-(2-{4-[(4-methoxy-phenyl)-methyl-amino]-6-methyl-quinazolin-2-ylamino}-ethyl)-acetamide;
(2-{4-[(4-methoxy-phenyl)-methyl-amino]-quinazolin-2-ylamino}-ethyl)-carbamic acid tert-butyl ester;
N-(2-{4-[(3,4-dimethoxy-phenyl)-methyl-amino]-quinazolin-2-ylamino}-ethyl)-acetamide;
(2-{4-[(3,4-dimethoxy-phenyl)-methyl-amino]-quinazolin-2-ylamino}-ethyl)-carbamic acid methyl ester;
(2-{6-methoxy-4-[(4-methoxy-phenyl)-methyl-amino]-quinazolin-2-ylamino}-ethyl)-urea;
(S)-2-{4-[(4-methoxy-phenyl)-methyl-amino]-quinazolin-2-ylamino}-2-phenyl-ethanol;
(1R,2S)-2-{4-[(4-methoxy-phenyl)-methyl-amino]-quinazolin-2-ylamino}-1-phenyl-propan-1-ol;
(S)-2-{4-[(4-methoxy-phenyl)-methyl-amino]-quinazolin-2-ylamino}-3-phenyl-propan-1-ol;
(2-{6-methoxy-4-[(4-methoxy-phenyl)-methyl-amino]-quinazolin-2-ylamino}-ethyl)-carbamic acid methyl ester;
(S)-2-amino-4-methylsulfanyl-butyric acid 2-{4-[(4-methoxy-phenyl)-methyl-amino]-quinazolin-2-ylamino}-ethyl ester;
(S)-2-{4-[(4-methoxy-phenyl)-methyl-amino]-quinazolin-2-ylamino}-propionic acid tert-butyl ester;
(S)-2-benzyloxycarbonylamino-3-(1H-indol-3-yl)-propionic acid 2-{4-[(4-methoxy-phenyl)-methyl-amino]-quinazolin-2-ylamino}-ethyl ester;
(2-{4-[(4-methoxy-phenyl)-methyl-amino]-6-methyl-quinazolin-2-ylamino}-ethyl)-carbamic acid methyl ester;
[(2-{4-[(4-methoxy-phenyl)-methyl-amino]-quinazolin-2-ylamino}-ethylcarbamoyl)-methyl]-carbamic acid tert-butyl ester;
2-{4-[(4-methoxy-phenyl)-methyl-amino]-quinazolin-2-ylamino}-3-methyl-butyric acid ethyl ester;
(S)-2-amino-succinamic acid 2-{4-[(4-methoxy-phenyl)-methyl-amino]-quinazolin-2-ylamino}-ethyl ester;
(S)-3-(4-hydroxy-phenyl)-2-{4-[(4-methoxy-phenyl)-methyl-amino]-quinazolin-2-ylamino}-propionic acid methyl ester;
(R)-2-amino-3-(1H-indol-3-yl)-propionic acid 2-{4-[(4-methoxy-phenyl)-methyl-amino]-quinazolin-2-ylamino}-ethyl ester;
(S)-2-{4-[(4-methoxy-phenyl)-methyl-amino]-quinazolin-2-ylamino}-propionic acid;
1-(2-{6-methoxy-4-[(4-methoxy-phenyl)-methyl-amino]-quinazolin-2-ylamino}-ethyl)-3-methyl-urea;
(2-{4-[(4-methoxy-phenyl)-methyl-amino]-quinazolin-2-ylamino}-ethyl)-carbamic acid methyl ester;
N-[2-(acetyl-{4-[(4-methoxy-phenyl)-methyl-amino]-quinazolin-2-yl}-amino)-ethyl]-acetamide;
(2-{4-[(4-methoxy-phenyl)-methyl-amino]-quinazolin-2-ylamino}-ethyl)-urea;
2-Amino-N-(2-{4-[(4-methoxy-phenyl)-methyl-amino]-quinazolin-2-ylamino}-ethyl)-acetamide;
1,3-bis-{4-[(4-methoxy-phenyl)-methyl-amino]-quinazolin-2-ylamino}-propan-2-ol;
{6-fluoro-4-[(4-methoxy-phenyl)-methyl-amino]-quinazolin-2-ylmethyl}-carbamic acid phenyl ester;
1-amino-3-{4-[(4-methoxy-phenyl)-methyl-amino]-quinazolin-2-ylamino}-propan-2-ol;
4-methoxy-N-{4-[(4-methoxy-phenyl)-methyl-amino]-quinazolin-2-ylmethyl}-benzenesulfonamide;
{4-[(4-methoxy-phenyl)-methyl-amino]-quinazolin-2-ylmethyl}-carbamic acid phenyl ester;
{4-[(4-methoxy-phenyl)-methyl-amino]-quinazolin-2-ylmethyl}-carbamic acid benzyl ester;
3-{4-[(4-methoxy-phenyl)-methyl-amino]-quinazolin-2-ylamino}-1-({4-[(4-methoxy-phenyl)-methyl-amino]-quinazolin-2-yl}-methyl-amino)-propane;
isobutyric acid 1-{4-[(4-methoxy-phenyl)-methyl-amino]-quinazolin-2-ylmethylcarbamoyloxy}-ethyl ester;
[1-({4-[(4-methoxy-phenyl)-methyl-amino]-quinazolin-2-ylmethyl}-carbamoyl)-ethyl]-carbamic acid tert-butyl ester;
[(S)-2-carbamoyl-1-({4-[(4-methoxy-phenyl)-methyl-amino]-quinazolin-2-ylmethyl}-carbamoyl)-ethyl]-carbamic acid tert-butyl ester;
(S)-2-amino-N¹-{4-[(4-methoxy-phenyl)-methyl-amino]-quinazolin-2-ylmethyl}-succinamide;
[(R)-1-({4-[(4-methoxy-phenyl)-methyl-amino]-quinazolin-2-ylmethyl}-carbamoyl)-ethyl]-carbamic acid tert-butyl ester;
3-(bis-{4-[(4-methoxy-phenyl)-methyl-amino]-quinazolin-2-ylmethyl}-amino)-propan-1-ol; and
acetic acid 3-acetoxy-2-{4-[(4-methoxy-phenyl)-methyl-amino]-quinazolin-2-ylmethylcarbamoyloxy}-propyl ester.

With the exception of example 48, all compounds were named using the AutoNom 2000 (MDL Information Systems) add-in for MDL ISIS™/Draw 2.5 SP 1 (Symyx Technologies, Inc., Sunnyvale, Calif.).

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

The term “alkyl” as employed herein by itself or as part of another group refers to both straight and branched chain radicals of up to ten carbons. Useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tent-butyl, 3-pentyl, hexyl and octyl groups, which may be optionally substituted.

The term “alkenyl” as employed herein by itself or as part of another group means a straight or branched chain radical of 2-10 carbon atoms, unless the chain length is limited thereto, including at least one double bond between two of the carbon atoms in the chain. Typical alkenyl groups include ethenyl, 1-propenyl, 2-propenyl, 2-methyl-1-propenyl, 1-butenyl and 2-butenyl.

The term “alkynyl” is used herein to mean a straight or branched chain radical of 2-10 carbon atoms, unless the chain length is limited thereto, wherein there is at least one triple bond between two of the carbon atoms in the chain. Typical alkynyl groups include ethynyl, 1-propynyl, 1-methyl-2-propynyl, 2-propynyl, 1-butynyl and 2-butynyl.

Useful alkoxy groups include oxygen substituted by one of the C₁₋₁₀ alkyl groups mentioned above, which may be optionally substituted.

Useful alkylthio groups include sulfur substituted by one of the C₁₋₁₀ alkyl groups mentioned above, which may be optionally substituted. Also included are the sulfoxides and sulfones of such alkylthio groups.

Useful amino groups include NH₂, NHRₓ and NRₓR_{y}, wherein Rₓ and R_{y} are C₁₋₁₀ alkyl or cycloalkyl groups. The alkyl group may be optionally substituted.

Optional substituents include one or more substituents chosen from hydroxyl, halo, alkyl, alkenyl, alkynyl, alkoxy, haloalkyl, haloalkoxy, amino, —C(═O)OH, —C(═O)O(C₁₋₃ alkyl), C₁₋₆ alkyl-C(═O)O(C₁₋₃ alkyl), C₁₋₆ alkyl-C(═O)OH, C₁₋₆ alkyl-C(═O)NH(C₁₋₃ alkyl), C₁₋₆ alkyl-C(═O)N(C₁₋₃ alkyl)₂, —C(═O)NH₂, —C(═O)NH(C₁₋₃ alkyl), —C(═O)N(C₁₋₃alkyl)₂, —S(═O)₂(C₁₋₃alkyl), —S(═O)₂NH₂, —S(═O)₂N(C₁₋₃alkyl)₂, —S(═O)₂NH(C₁₋₃ alkyl), —SH, —SCF₃, —CN, —NH₂, and —NO₂.

The term “aryl” as employed herein by itself or as part of another group refers to monocyclic, bicyclic or tricyclic aromatic groups containing from 6 to 14 carbons in the ring portion.

Useful aryl groups include C₆₋₁₄ aryl, preferably C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

The term “carbocycle” as employed herein include cycloalkyl and partially saturated carbocyclic groups. Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as described above, as well as cycloalkenyl groups, such as cyclopentenyl, cycloheptenyl and cyclooctenyl.

Useful halo or halogen groups include fluorine, chlorine, bromine and iodine.

The term “arylalkyl” is used herein to mean any of the above-mentioned C₁₋₁₀ alkyl groups substituted by any of the above-mentioned C₆₋₁₄ aryl groups. Preferably the arylalkyl group is benzyl, phenethyl or naphthylmethyl.

The term “arylalkenyl” is used herein to mean any of the above-mentioned C₂₋₁₀ alkenyl groups substituted by any of the above-mentioned C₆₋₁₄ aryl groups.

The term “arylalkynyl” is used herein to mean any of the above-mentioned C₂₋₁₀ alkynyl groups substituted by any of the above-mentioned C₆₋₁₄ aryl groups.

The term “aryloxy” is used herein to mean oxygen substituted by one of the above-mentioned C₆₋₁₄ aryl groups, which may be optionally substituted. Useful aryloxy groups include phenoxy and 4-methylphenoxy.

The term “arylalkoxy” is used herein to mean any of the above mentioned C₁₋₁₀ alkoxy groups substituted by any of the above-mentioned aryl groups, which may be optionally substituted. Useful arylalkoxy groups include benzyloxy and phenethyloxy.

The term “haloalkyl” is used herein to mean C₁₋₁₀ alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino (acylamido) groups are any C₁₋₆ acyl (alkanoyl) attached to an amino nitrogen, e.g., acetamido, chloroacetamido, propionamido, butanoylamido, pentanoylamido and hexanoylamido, as well as aryl-substituted C₁₋₆ acylamino groups, e.g., benzoylamido, and pentafluorobenzoylamido.

Useful acyloxy groups are any C₁₋₆ acyl (alkanoyl) attached to an oxy (—O—) group, e.g., formyloxy, acetoxy, propionoyloxy, butanoyloxy, pentanoyloxy and hexanoyloxy.

The term heterocycle is used herein to mean a saturated or partially saturated 3-7 membered monocyclic, or 7-10 membered bicyclic ring system, which consists of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on a carbon or on a nitrogen atom if the resulting compound is stable, including an oxo substituent (“═O”) wherein two hydrogen atoms are replaced.

Useful saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

The term “heteroaryl” as employed herein refers to groups having 5 to 14 ring atoms; 6, 10 or 14 π electrons shared in a cyclic array; and containing carbon atoms and 1, 2 or 3 oxygen, nitrogen or sulfur heteroatoms.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

The term “heteroaryloxy” is used herein to mean oxygen substituted by one of the above-mentioned heteroaryl groups, which may be optionally substituted. Useful heteroaryloxy groups include pyridyloxy, pyrazinyloxy, pyrrolyloxy, pyrazolyloxy, imidazolyloxy and thiophenyloxy.

The term “heteroarylalkoxy” is used herein to mean any of the above-mentioned C₁₋₁₀ alkoxy groups substituted by any of the above-mentioned heteroaryl groups, which may be optionally substituted.

Some of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

Examples of pharmaceutically acceptable addition salts include inorganic and organic acid addition salts, and inorganic and organic base addition salts with bases.

Examples of prodrugs of the compounds of the invention include the simple esters of carboxylic acid containing compounds; esters of hydroxy containing compounds; imines of amino containing compounds; carbamate of amino containing compounds; and acetals and ketals of alcohol containing compounds.

In the compounds of the invention, reference to any bound hydrogen atom can also encompass a deuterium atom bound at the same position. Substitution of hydrogen atoms with deuterium atoms is conventional in the art. See, e.g., U.S. Pat. Nos. 5,149,820 & 7,317,039. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms dramatically slows the catabolism of the deuterated compound, relative to the non-deuterated compound, such that the deuterated compound exhibit a significantly longer half-life in the bodies of individuals administered such compounds. This is particularly so when the catabolism of the hydrogenated compound is mediated by cytochrome P450 systems. Kushner et al., Can. J. Physiol. Pharmacol. (1999) 77:79-88.

The compounds of this invention may be prepared using methods known to those skilled in the art, or the novel methods of this invention. In one embodiment of a process for the manufacture of a compound according to Formula I the method comprises reacting a first compound of the following formula:

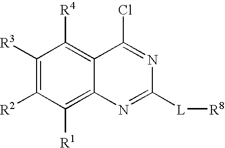

wherein R₁, R₂, R₃, R₄, L, and R₈ are as defined in Formula I previously;
with a second compound of the following formula:

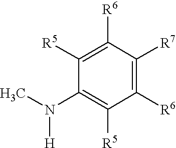

wherein R₅, R₆, and R₇ are as defined in Formula I previously, to form a product according to Formula I. In additional embodiments, the compounds of this invention with Formula I can be prepared as illustrated by the exemplary reactions in Schemes 1-7.

Appropriately substituted nucleophiles can be obtained as shown in scheme 1. The aniline precursor can be homologated through reductive amination with formaldehyde or other appropriate aldehydes or ketones. Numerous reducing agents can be employed, including, but not limited to sodium borohydride, sodium cyanoborohydride, lithium aluminum hydride, borane, etc.

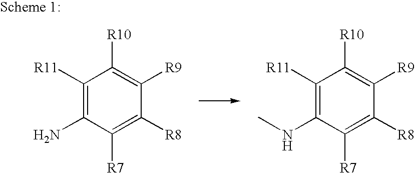

As shown in scheme 2, an appropriately substituted anthranilic acid can be converted to a benzoyleneurea. Treatment with a chlorinating reagent (phosphorus oxychloride, sulfuryl chloride, thionyl chloride or the like) yields the corresponding dichloroquinazoline. The appropriately substituted nucleophile (from scheme 1 or any other) can be used to displace one chloro group on the dichloroquinazoline. The remaining chloride can be displaced with a second nucleophile (including, but not limited to, anilines, amines, alcohols, ethanolamine, potassium cyanide, ethylenediamine, metal alkoxide, and the like).

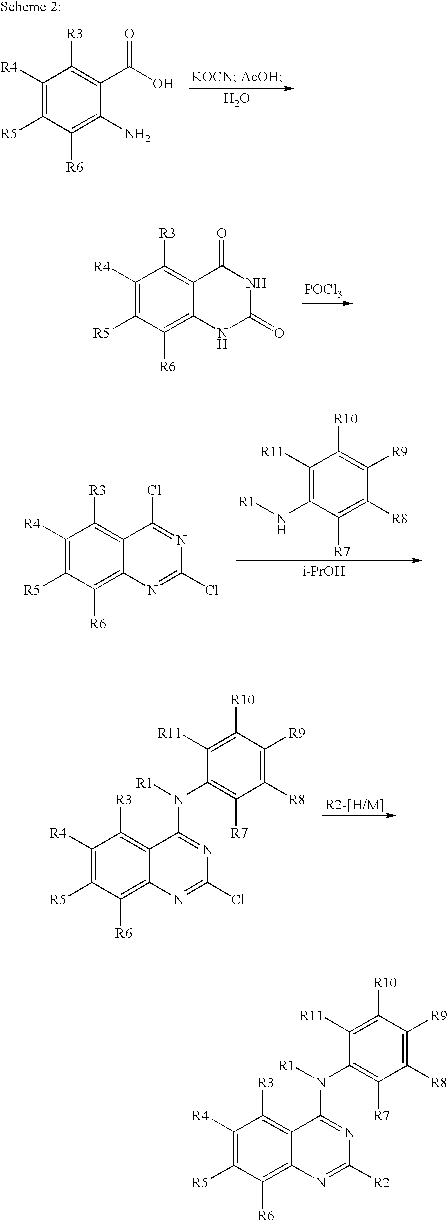

As depicted in Scheme 3, treatment of a substituted anthranilic ester with a substituted nitrile under acidic conditions, yields the 2-substituted quinazolinone. Chlorination of the quinazolinone with phosphorus oxychloride results in a 4-chloroquinazoline. Displacement with an appropriately substituted nucleophile (aniline, amine, alcohol, metal alkoxide, etc.) leads to compounds of Formula I.

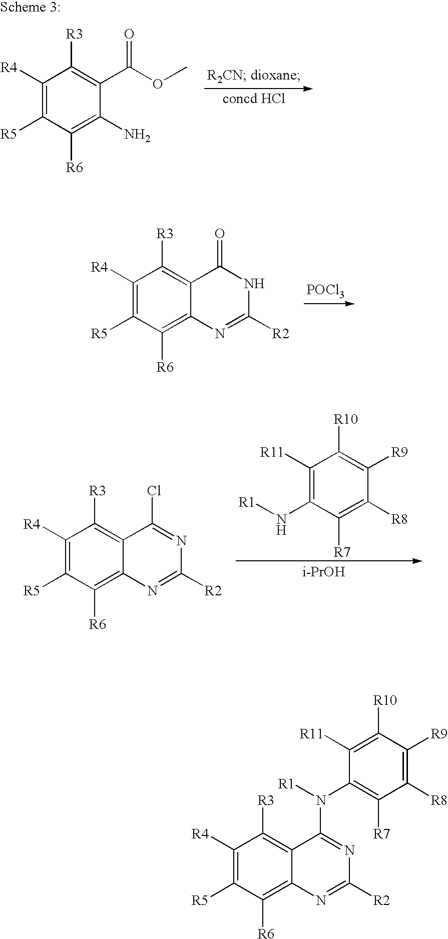

Similar to above (scheme 3) scheme 4 depicts that an appropriately substituted anthranilic ester can be converted to a dichloroquinazolinone via treatment with a chloro-substituted nitrile. The two dichloroquinazoline can be sequentially substituted with nucleophiles. The phthalimide protecting group can be removed to reveal an amine which can undergo further substitution to give compounds of Formula 1.

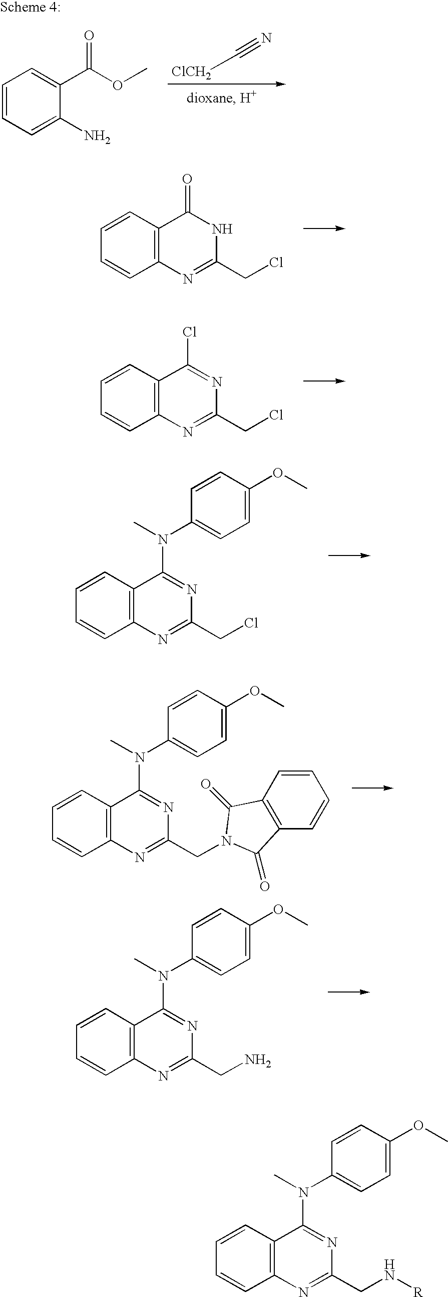

Similar to above (scheme 3) scheme 5 depicts that an appropriately substituted anthranilic ester can be converted to the quinazolinone via treatment with a substituted nitrile. For instance, using chloroacetonitrile the resultant quinazoline can be further modified through treatment with nucleophiles. Under appropriate conditions, dimer-like structures can be formed as shown in scheme 5.

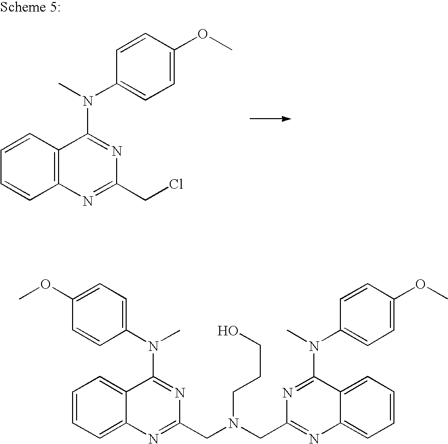

Dimeric and monomeric species can also be obtained from the 2-chloroquinazoline as outlined in scheme 6. Treating an appropriately substituted 2-chloroquinazoline with a bisnucleophile (for example but not limited to, diamine, aminoalcohol) species can be obtained as shown in scheme 6.

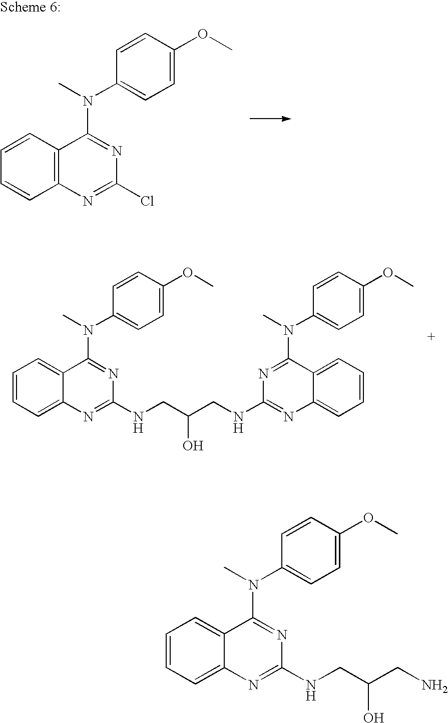

A quinazoline methylamine as shown in scheme 7 can be further elaborated through acylation and further extension to give compounds of Formula 1.

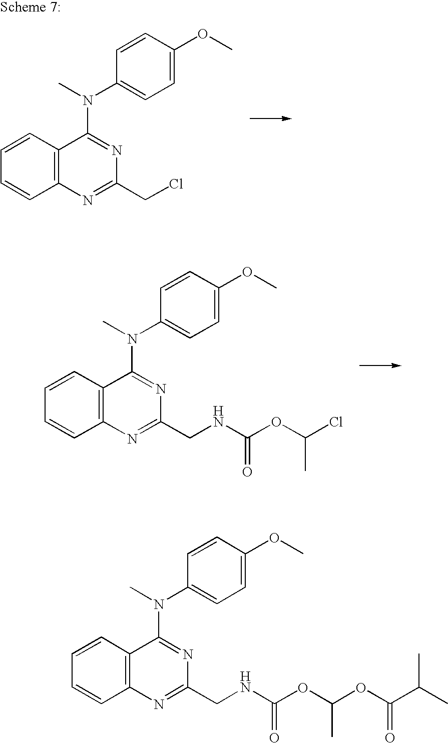

In the above Schemes, where R2 contains reactive functionalities it can be further transformed; for instance, an amine can be transformed into an amide, urea or carbamate; a carbamate can be cleaved to an amine; a nitrile can be reduced; etc.

An important aspect of the present invention is the discovery that compounds having Formula I are cytotoxic agents. Therefore, these compounds are useful in treating diseases that are responsive to cytotoxic agents. For example, these compounds are useful in a variety of clinical conditions in which there is uncontrolled cell growth and spread of abnormal cells, such as in the case of cancer.

The present invention also includes a therapeutic method comprising administering to an animal an effective amount of a compound, or a pharmaceutically acceptable salt or prodrug of said compound of Formula I, wherein said therapeutic method is useful to treat cancer, which is a group of diseases characterized by the uncontrolled growth and spread of abnormal cells.

In practicing the therapeutic methods, effective amounts of compositions containing therapeutically effective concentrations of the compounds formulated for oral, intravenous, local and topical application, for the treatment of neoplastic diseases and other diseases, are administered to an individual exhibiting the symptoms of one or more of these disorders. The amounts are effective to ameliorate or eliminate one or more symptoms of the disorders. An effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the symptoms of the disease. Typically, repeated administration is required to achieve the desired amelioration of symptoms.

Another aspect of the present invention is to provide a pharmaceutical composition, containing an effective amount of a compound of Formula I, or a pharmaceutically acceptable salt of said compound, in admixture with one or more pharmaceutically acceptable carriers or diluents.

In one embodiment, a pharmaceutical composition comprising a compound of Formula I disclosed herein, or a pharmaceutically acceptable salt of said compound, in combination with a pharmaceutically acceptable vehicle is provided.

Preferred pharmaceutical compositions comprise compounds of Formula I, and pharmaceutically acceptable salts, esters, or prodrugs thereof, that are cytotoxic as determined by the method described in Example 14, preferably at an EC₅₀ no greater than 1,000 nM, more preferably at an EC₅₀ no greater than 500 nM, more preferably at an EC₅₀ no greater than 200 nM, more preferably at an EC₅₀ no greater than 100, and most preferably at an EC₅₀ no greater than 10 nM.

Another embodiment of the present invention is directed to a composition effective to inhibit neoplasia comprising a compound, or a pharmaceutically acceptable salt or prodrug of said compound of Formula I, which functions as a cytotoxic agent, in combination with at least one known cancer chemotherapeutic agent, or a pharmaceutically acceptable salt of said agent. Examples of known cancer chemotherapeutic agents which may be used for combination therapy include, but not are limited to alkylating agents, antimitotic agents, topoisomerase I inhibitors, topoisomerase II inhibitors, RNA/DNA antimetabolites, DNA antimetabolites, EGFR inhibitors, proteosome inhibitors, and antibodies.

In practicing the methods of the present invention, the compound of the invention may be administered together with at least one known chemotherapeutic agent as part of a unitary pharmaceutical composition. Alternatively, the compound of the invention may be administered apart from at least one known cancer chemotherapeutic agent. In one embodiment, the compound of the invention and at least one known cancer chemotherapeutic agent are administered substantially simultaneously; i.e. the compounds are administered at the same time or one after the other, so long as the compounds reach therapeutic levels in the blood at the same time. On another embodiment, the compound of the invention and at least one known cancer chemotherapeutic agent are administered according to their individual dose schedule, so long as the compounds reach therapeutic levels in the blood.

It has been reported that alpha-1-adrenoceptor antagonists can inhibit the growth of prostate cancer cell via induction of apoptosis (Kyprianou, N., et al., Cancer Res 60:4550-4555, (2000)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known alpha-1-adrenoceptor antagonists, or a pharmaceutically acceptable salt of said agent.

It has been reported that sigma-2 receptors are expressed in high densities in a variety of tumor cell types (Vilner, B. J., et al., Cancer Res. 55: 408-413 (1995)) and that sigma-2 receptor agonists activate a novel apoptotic pathway and potentiate antineoplastic drugs in breast tumor cell lines. (Kyprianou, N., et al., Cancer Res. 62:313-322 (2002)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known sigma-2 receptor agonist, or a pharmaceutically acceptable salt of said agonist.

It has been reported that combination therapy with a HMG-CoA reductase inhibitor, and butyrate, an inducer of apoptosis in the Lewis lung carcinoma model in mice, showed potentiating antitumor effects (Giermasz, A., et al., Int. J. Cancer 97:746-750 (2002)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known HMG-CoA reductase inhibitor, or a pharmaceutically acceptable salt of said agent.

It has been reported that HIV protease inhibitors have potent anti-angiogenic activities and promote regression of Kaposi sarcoma (Sgadari, C., et al., Nat. Med. 8:225-232 (2002)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known HIV protease inhibitor, or a pharmaceutically acceptable salt of said agent.

It has been reported that synthetic retinoids have good activity in combination with other chemotherapeutic agents in small-cell lung cancer cell lines (Kalemkerian, G. P., et al., Cancer Chemother. Pharmacol. 43:145-150 (1999)). Synthetic retinoids have also been reported to have good activity in combination with gamma-radiation on bladder cancer cell lines (Zou, C., et al., Int. J. Oncol. 13:1037-1041 (1998)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known retinoid and synthetic retinoid, or a pharmaceutically acceptable salt of said agent.

It has been reported that proteasome inhibitors exert anti-tumor activity in vivo and in tumor cells in vitro, including those resistant to conventional chemotherapeutic agents. By inhibiting NF-kappaB transcriptional activity, proteasome inhibitors may also prevent angiogenesis and metastasis in vivo and further increase the sensitivity of cancer cells to apoptosis (Almond, J. B., et al., Leukemia 16:433-443 (2002)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known proteasome inhibitor, or a pharmaceutically acceptable salt of said agent.

It has been reported that tyrosine kinase inhibitors have potent synergetic effect in combination with other anti-leukemic agents (Liu, W. M., et al. Br. J. Cancer 86:1472-1478 (2002)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known tyrosine kinase inhibitor, or a pharmaceutically acceptable salt of said agent.

It has been reported that prenyl-protein transferase inhibitors possess preclinical antitumor activity against human breast cancer (Kelland, L. R., et. al., Clin. Cancer Res. 7:3544-3550 (2001)). Synergy of a protein farnesyltransferase inhibitor and cisplatin in human cancer cell lines also has been reported (Adjei, A. A., et al., Clin. Cancer. Res. 7:1438-1445 (2001)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known prenyl-protein transferase inhibitor, including farnesyl protein transferase inhibitor, inhibitors of geranylgeranyl-protein transferase type I (GGPTase-I) and geranylgeranyl-protein transferase type-II, or a pharmaceutically acceptable salt of said agent.

It has been reported that cyclin-dependent kinase (CDK) inhibitors have potent synergetic effect in combination with other anticancer agents, such as a DNA topoisomerase I inhibitor in human colon cancer cells (Motwani, M., et al., Clin. Cancer Res. 7:4209-4219, (2001)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known cyclin-dependent kinase inhibitor, or a pharmaceutically acceptable salt of said agent.

It has been reported that in preclinical studies COX-2 inhibitors were found to block angiogenesis, suppress solid tumor metastases, and slow the growth of implanted gastrointestinal cancer cells (Blanke, C. D., Oncology (Huntingt) 16(No. 4 Suppl. 3):17-21 (2002)). Therefore, another embodiment of the present invention is directed to a composition effective to inhibit neoplasia comprising a compound, or a pharmaceutically acceptable salt or prodrug of a compound described herein, which functions as a cytotoxic agent, in combination with at least one known COX-2 inhibitor, or a pharmaceutically acceptable salt of said inhibitor.

Another embodiment of the present invention is directed to a composition effective to inhibit neoplasia comprising a bioconjugate of a compound described herein, which functions as a cytotoxic agent, in bioconjugation with at least one known therapeutically useful antibody, growth factors, cytokines, or any molecule that binds to the cell surface. The antibodies and other molecules will deliver a compound described herein to its targets and make it an effective anticancer agent. The bioconjugates could also enhance the anticancer effect of therapeutically useful antibodies.

Similarly, another embodiment of the present invention is directed to a composition effective to inhibit neoplasia comprising a compound, or a pharmaceutically acceptable salt or prodrug of a compound described herein, which functions as a cytotoxic agent, in combination with radiation therapy. In this embodiment, the compound of the invention may be administered at the same time as the radiation therapy is administered or at a different time.

Yet another embodiment of the present invention is directed to a composition effective for post-surgical treatment of cancer, comprising a compound, or a pharmaceutically acceptable salt or prodrug of a compound described herein, which functions as a cytotoxic agent. The invention also relates to a method of treating cancer by surgically removing the cancer and then treating the animal with one of the pharmaceutical compositions described herein.

Stent implantation has become the new standard angioplasty procedure. However, in-stent restenosis remains the major limitation of coronary stenting. New approaches have been developed to target pharmacological modulation of local vascular biology by local administration of drugs. This allows for drug applications at the precise site and time of vessel injury. Numerous pharmacological agents with antiproliferative properties are currently under clinical investigation, including actinomycin D, rapamycin or paclitaxel coated stents (Regar E., et al., Br. Med. Bull. 59:227-248 (2001)). Therefore, apoptosis inducers, which are antiproliferative, are useful as therapeutics for the prevention or reduction of in-stent restenosis.

Another important aspect of the present invention is the surprising discovery that compounds of the present invention are potent and highly efficacious cytotoxic agents even in drug resistant cancer cells, which enables these compounds to inhibit the growth and proliferation of drug resistant cancer cells, and to cause cell death in the drug resistant cancer cells. Specifically, the compounds of the present invention are not substrates for the MDR transporters such as Pgp-1 (MDR-1), MRP-1 and BCRP. This is particularly surprising in view of the fact that many commercially available chemotherapeutics are substrates for multidrug resistance transporters (MDRs).

Multidrug resistance is the major cause of chemotherapy failure. Drug resistance is typically caused by ATP-dependent efflux of drug from cells by ATP-binding cassette (ABC) transporters. In particular, the ABC transporters ABCB1 (MDR-1, P glycoprotein); ABCC1 (MRP1); and ABCG2 (BCRP, MXR) are typically over-expressed in drug resistant tumors and thus are implicated in drug resistance. In comparison to most standard anti-cancer drugs, which are not effective in killing drug resistant cancer cells, the compounds of the present invention are effective in killing drug resistant cancer cells. Therefore, compounds of this invention are useful for the treatment of drug resistant cancer.

Thus, another aspect of the present invention is the application of the methods and compounds of the present invention as described above to tumors that have acquired resistance to other anticancer drugs. In one embodiment, a compound of the present invention is administered to a cancer patient who has been treated with another anti-cancer drug. In another embodiment, a compound of the present invention is administered to a patient who has been treated with and is not responsive to another anti-cancer drug or developed resistance to such other anti-cancer compound. In another embodiment, a compound of the present invention is administered to a patient who has been treated with another anti-cancer drug and is refractory to said other anti-cancer drug. The compounds of the present invention can be used in treating cancer in a patient who is not responsive or is resistant to any other anti-cancer agent. Examples of such other anti-cancer agent may include alkylating agents, antimitotic agents, topoisomerase I inhibitors, topoisomerase II inhibitors, RNA/DNA antimetabolites, EGFR inhibitors, angiogenesis inhibitors, tubulin inhibitors, proteosome inhibitors, etc.

Pharmaceutical compositions within the scope of this invention include all compositions wherein the compounds of the present invention are contained in an amount that is effective to achieve its intended purpose. While individual needs vary, determination of optimal ranges of effective amounts of each component is within the skill of the art. Typically, the compounds may be administered to animals, e.g., mammals, orally at a dose of 0.0025 to 50 mg/kg of body weight, per day, or an equivalent amount of the pharmaceutically acceptable salt thereof, to a mammal being treated. Preferably, approximately 0.01 to approximately 10 mg/kg of body weight is orally administered. For intramuscular injection, the dose is generally approximately one-half of the oral dose. For example, a suitable intramuscular dose would be approximately 0.0025 to approximately 25 mg/kg of body weight, and most preferably, from approximately 0.01 to approximately 5 mg/kg of body weight. If a known cancer chemotherapeutic agent is also administered, it is administered in an amount that is effective to achieve its intended purpose. The amounts of such known cancer chemotherapeutic agents effective for cancer are well known to those skilled in the art.

The unit oral dose may comprise from approximately 0.01 to approximately 50 mg, preferably approximately 0.1 to approximately 10 mg of the compound of the invention. The unit dose may be administered one or more times daily, as one or more tablets, each containing from approximately 0.1 to approximately 10 mg, conveniently approximately 0.25 to 50 mg of the compound or its solvates.

In a topical formulation, the compound may be present at a concentration of approximately 0.01 to 100 mg per gram of carrier.

In addition to administering the compound as a raw chemical, the compounds of the invention may be administered as part of a pharmaceutical preparation containing suitable pharmaceutically acceptable carriers comprising excipients and auxiliaries, which facilitate processing of the compounds into preparations that may be used pharmaceutically. Preferably, the preparations, particularly those preparations which may be administered orally and that may be used for the preferred type of administration, such as tablets, dragees, and capsules, and also preparations that may be administered rectally, such as suppositories, as well as suitable solutions for administration by injection or orally, contain from approximately 0.01 to 99 percent, preferably from approximately 0.25 to 75 percent of active compound(s), together with the excipient.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid. Basic salts are formed by mixing a solution of the compounds of the present invention with a solution of a pharmaceutically acceptable non-toxic base.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use may be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular: fillers, cellulose preparations and/or calcium phosphates, as well as binders. If desired, disintegrating agents may be added, such as starches and also carboxymethyl-starch, cross-linked polyvinyl pyrrolidone, agar, or alginic acid or a salt thereof. Auxiliaries are, above all, flow-regulating agents and lubricants. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations are used. Dye stuffs or pigments may be added to the tablets or dragee coatings, e.g., for identification or in order to characterize combinations of active compound doses.

Other pharmaceutical preparations, which may be used orally include push-fit capsules made of gelatin, as well as soft, sealed capsules made of gelatin and a plasticizer. The push-fit capsules may contain the active compounds in the form of: granules, which may be mixed with fillers, binders, and/or lubricants, and, optionally, stabilizers. In soft capsules, the active compounds are preferably dissolved or suspended in suitable liquids, such as fatty oils, or liquid paraffin. In addition, stabilizers may be added.

Possible pharmaceutical preparations, which may be used rectally include, e.g., suppositories, which consist of a combination of one or more of the active compounds with a suppository base. Suitable suppository bases are, e.g., natural or synthetic triglycerides, or paraffin hydrocarbons. In addition, it is also possible to use gelatin rectal capsules, which consist of a combination of the active compounds with a base. Possible base materials include, e.g., liquid triglycerides, polyethylene glycols, or paraffin hydrocarbons.

Suitable formulations for parenteral administration include aqueous solutions of the active compounds in water-soluble form, e.g., water-soluble salts and alkaline solutions. In addition, suspensions of the active compounds as appropriate oily injection suspensions may be administered. Suitable lipophilic solvents or vehicles include fatty oils or synthetic fatty acid esters. Aqueous injection suspensions may contain substances which increase the viscosity of the suspension include. Optionally, the suspension may also contain stabilizers.

In accordance with one aspect of the present invention, compounds of the invention are employed in topical and parenteral formulations and are used for the treatment of skin cancer.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention. In the following examples, all nuclear magnetic resonance spectra were obtained at a frequency of 400 MHz. Typical preparative RPLC purification conditions, where applicable, involve a C-18 stationary phase and a gradient of (0.01% TFA in acetonitrile) in (0.01% TFA in water).

### EXAMPLES

### Example 1

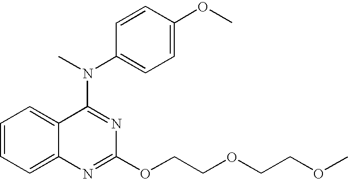

### {2-[2-(2-Methoxy-ethoxy)-ethoxy]-quinazolin-4-yl}-(4-methoxy-phenyl)-methyl-amine

2,4-Dichloro-quinazoline: A suspension of 1H-quinazoline-2,4-dione (10 g, 62 mmol), POCl₃ (50 mL, 546 mmol) and N,N-dimethylaniline (1 mL, 7.9 mmol) was heated to reflux for 18 h. The reaction mixture was cooled to room temperature and poured slowly onto ice and extracted with CH₂Cl₂. The combined extracts were filtered through Na₂SO₄ and concentrated to give 4.2 g (34%) of 2,4-dichloro-quinazoline as a white solid.

(2-Chloro-quinazolin-4-yl)-(4-methoxy-phenyl)-methyl-amine hydrochloride: A solution of 2,4-dichloro-quinazoline (1 g, 5 mmol) and (4-methoxy-phenyl)-methyl-amine (0.823 g, 6 mmol) in i-PrOH (17 mL) with concd HCl (10 drops) was stirred at room temperature overnight. The reaction was filtered, washed with i-PrOH and dried under vacuum to provide 1 g (66%) of the title compound as a white solid. ¹H NMR (CDCl₃) δ 8.65 (d, 1H), 7.7 (t, 1H), 7.23 (d, 2H), 7.18 (t, 1H), 7.08 (d, 2H), 6.75 (d, 1H), 3.9 (s, 3H) 3.8 (s, 3H).

{2-[2-(2-M ethoxy-ethoxy)-ethoxy]-quinazolin-4-yl}-(4-methoxy-phenyl)-methyl-amine: NaH (60% dispersion, 30.0 mg, 0.75 mmol) was placed in a 25 mL roundbottom flask under a blanket of inert gas, and washed three times with hexanes. After decanting the last hexanes wash, the residue was dried with flowing N₂ gas. The residue was then treated with 2 mL of di(ethylene glycol)-monomethyl ether, after which (2-chloro-quinazolin-4-yl)-(4-methoxy-phenyl)-methyl-amine hydrochloride (100 mg, 0.3 mmol) was added in 1 mL of DMF. The mixture was heated at 100° C. with stirring for two hours. The mixture was cooled, diluted with ethyl acetate (˜10 mL), and washed with water. The organic layer was dried over anhydrous sodium sulfate and concentrated, yielding the title compound (114 mg; 90%). ¹H NMR (CDCl₃) δ 7.59 (d, 1H, J=9 Hz), 7.46 (t, 1H, J=9 Hz), 7.10-7.14 (m, 2H), 6.87-6.96 (m, 3H), 6.83 (t, 1H, J=9 Hz), 4.66 (t, 2H, J=6.4 Hz), 3.98 (t, 2H, J=6.4 Hz), 3.84 (s, 3H), 3.74-3.78 (m, 2H), 3.58-3.61 (m, 2H), 3.57 (s, 3H), 3.40 (s, 3H); LC-MS (ESI⁺; 384 ([M+H]⁺).

### Example 2

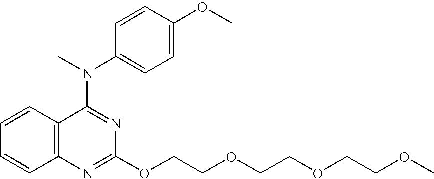

### (2-{2-[2-(2-Methoxy-ethoxy)-ethoxy]-ethoxy}-quinazolin-4-yl)-(4-methoxy-phenyl)-methyl-amine

The procedure found in Example 1 was used to prepare the title compound (127 mg, 90%). To remove excess solvent (triethylene glycol monomethyl ether), the stripped organic layer was placed on high vacuum for several days. ¹H NMR (CDCl₃) δ 7.59 (d, 1H, J=9 Hz), 7.46 (t, 1H, J=9 Hz), 7.10-7.15 (m, 2H), 6.87-6.96 (m, 3H), 6.83 (t, 1H, J=9 Hz), 4.65 (t, 2H, J=5.5 Hz), 3.95 (t, 2H, J=5.1 Hz), 3.84 (s, 3H), 3.75-3.79 (m, 2H), 3.65-3.72 (m, 4H), 3.57 (s, 3H), 3.53-3.57 (m, 2H), 3.38 (s, 3H); LC-MS (ESI⁺; 428 ([M+H]⁺).

### Example 3

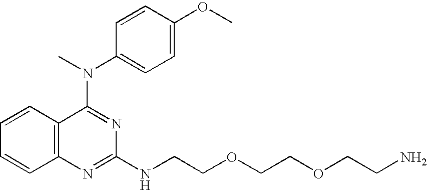

### N²-{2-[2-(2-Amino-ethoxy)-ethoxy]-ethyl}-N⁴-(4-methoxy-phenyl)-N⁴-methyl-quinazoline-2,4-diamine

(2-Chloro-quinazolin-4-yl)-(4-methoxy-phenyl)-methyl-amine hydrochloride (200 mg, 0.67 mmol) was placed in 3 mL of 2-[2-(2-amino-ethoxy)-ethoxy]-ethylamine and heated at 100° C. for 3 hours. The crude reaction mixture was loaded onto a 40 g silica cartridge and flushed with 5-10% acetone/hexanes (˜250 mL), then the pure product was eluted with 10% MeOH/1% TEA/chloroform to yield the title compound. ¹H NMR (CDCl₃) δ 7.35 (d, 1H, J=8.3 Hz), 7.23-7.30 (m, 1H), 6.97-7.04 (m, 2H), 6.76-6.83 (m, 3H), 6.58 (t, 1H, J=7.9 Hz), 3.72 (s, 3H), 3.55-3.63 (m, 4H), 3.55-3.63 (m, 4H), 3.43-3.47 (m, 4H), 3.41 (s, 3H), 2.80 (t, 2H, J=5.3 Hz).

### Example 4

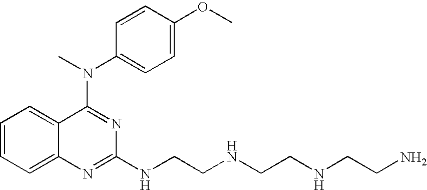

### N²-{2-[2-(2-Amino-ethylamino)-ethylamino]-ethyl}-N⁴-(4-methoxy-phenyl)-N⁴-methyl-quinazoline-2,4-diamine

The title compound was synthesized in a manner analogous to that used in Example 3. LC-MS (ESI⁻; 408 ([M−H]⁻).

### Example 5

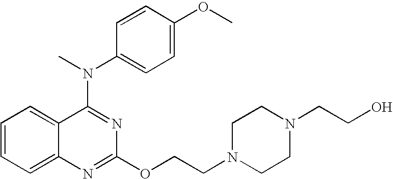

### 2-[4-(2-{4-[(4-Methoxy-phenyl)-methyl-amino]-quinazolin-2-yloxy}-ethyl)-piperazin-1-yl]-ethanol

NaH (60% dispersion, 100 mg, 2.5 mmol) was placed in a 25 mL roundbottom flask under a blanket of inert gas and washed three times with hexanes. After decanting the last hexanes wash, the residue was dried with flowing N₂ gas. A slurry of NaH was made in 3 mL THF. To this was added 2-[4-(2-hydroxy-ethyl)-piperazin-1-yl]-ethanol (871 mg, 10 eq.) in 3 mL DMF, then (2-chloro-quinazolin-4-yl)-(4-methoxy-phenyl)-methyl-amine hydrochloride (150 mg, 0.5 mmol). The mixture was stirred at 65° C. in a sealed vial for 2 hours. The mixture was cooled, diluted with ethyl acetate (˜10 mL) and washed with water. The organic layer was separated and passed through a 1 g silica cartridge, stripped, dissolved in DMSO (˜1 mL), passed through a 0.45 μm filter, then purified by preparative RPLC, to yield the title compound. ¹H NMR (CDCl₃) δ 7.60 (d, 1H, J=8.2 Hz), 7.43-7.49 (m, 1H), 7.13 (d, 2H, J=9.0 Hz), 6.89-6.96 (m, 3H), 6.80-6.86 (m, 1H), 4.63 (t, 2H, J=6.5 Hz), 3.84 (s, 3H), 3.62 (t, 2H, J=5.5 Hz), 3.58 (s, 3H), 2.92 (t, 2H, J=6.4 Hz), 2.58-2.75 (m, 6H), 2.56 (t, 2H, J=5.4 Hz).

### Example 6

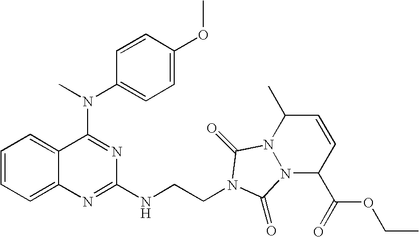

### 2-(2-{4-[(4-Methoxy-phenyl)-methyl-amino]-quinazolin-2-ylamino}-ethyl)-8-methyl-1,3-dioxo-2,3,5,8-tetrahydro-1H-[1,2,4]triazolo[1,2-a]pyridazine-5-carboxylic acid ethyl ester

8-Methyl-1,3-dioxo-2,3,5,8-tetrahydro-1-[1,2,4]triazolo[1,2-a]pyridazine-5-carboxylic acid ethyl ester: To a solution of urazole (0.202 g, 2.0 mmol) in DMF (4 mL) was added iodobenzene diacetate (0.86 g, 2.0 mmol) at 23° C., and the mixture was stirred at 23° C. for 10 minutes. After addition of ethyl sorbate (0.28 g, 2.0 mmol), the reaction mixture was stirred at 23° C. for 4 h. The precipitate was formed by addition of hexanes, and the crude product was further purified by recrystallization in chloroform/hexanes to give the title compound (0.30 g) in 63% yield. ¹H NMR (CD₃OD) δ 9.52 (s, 1H), 5.96-6.00 (m, 1H), 5.79-5.82 (m, 1H), 4.94-4.97 (m, 1H), 4.31-4.37 (m, 1H), 4.20-4.27 (m, 2H), 1.60 (d, J=6.7 Hz, 3H), 1.28 (t, J=7.2 Hz, 3H); HRMS m/z 240.1088 [M+H]⁺.

2-{4-[(4-Methoxy-phenyl)-methyl-amino]-quinazolin-2-ylamino}-ethanol: A mixture of (2-chloro-quinazolin-4-yl)-(4-methoxy-phenyl)-methyl-amine hydrochloride (0.336 g; 1.0 mmol), ethanolamine (0.183 g; 3.0 mmol) and Et₃N (0.1 g; 1.0 mmol) in isopropanol (5 mL) was stirred at 85° C. for 48 h. The reaction mixture was concentrated, EtOAc was added and this was washed with water, then dried over anhydrous MgSO₄. The crude product was then purified by MPLC (SiO₂, 0 to 100% EtOAc in hexanes) to give the title compound as a yellowish oil (0.26 g; 80%). ¹H NMR (CDCl₃) δ 7.34-7.42 (m, 2H), 7.07-7.10 (m, 2H), 6.87-6.91 (m, 2H), 6.83-6.85 (m, 1H), 6.66-6.70 (m, 1H), 3.89-3.91 (m, 2H), 3.82 (s, 3H), 3.65-3.68 (m, 2H), 3.48 (s, 3H); HRMS m/z 325.1657 [M+H]⁺.

2-(2-{4-[(4-Methoxy-phenyl)-methyl-amino]-quinazolin-2-ylamino}-ethyl)-8-methyl-1,3-dioxo-2,3,5,8-tetrahydro-1-[1,2,4]triazolo[1,2-a]pyridazine-5-carboxylic acid ethyl ester: A mixture of 2-{4-[(4-methoxy-phenyl)-methyl-amino]-quinazolin-2-ylamino}-ethanol (0.160 g, 0.5 mmol), 8-methyl-1,3-dioxo-2,3,5,8-tetrahydro-1-[1,2,4]triazolo[1,2-a]pyridazine-5-carboxylic acid ethyl ester (0.176 g, 0.6 mmol), DEAD (40% in toluene; 0.435 g, 1.0 mmol), Ph₃P (0.264 g, 1.0 mmol) and Et₃N (1.0 mL, 7.2 mmol) in THF (10 mL) was stirred at 23° C. for 16 h. The reaction mixture was directly purified with silica gel gradient column chromatography (EtOAc/hexanes, 0% to 100%) to give the crude product in 50% yield, which was further purified with preparative RPLC. ¹H NMR (CD₃OD) δ 7.63-7.56 (m, 1H), 7.40-7.30 (m, 3H), 7.09-7.00 (m, 3H), 6.95-6.88 (m, 1H), 6.11-6.07 (m, 1H), 6.03-5.98 (m, 1H), 5.08 (apparent d, 1H), 4.55-4.45 (m, 2H), 4.23 (q, 2H, J=7.0 Hz), 3.90-3.80 (m, 2H), 3.87 (s, 3H), 3.85 (s, 3H), 3.68 (d, 3H, J=8.2 Hz), 3.65-3.58 (m, 2H), 1.29-1.23 (m, 3H); HRMS m/z 546.2457 [M+H]⁺.

### Example 7

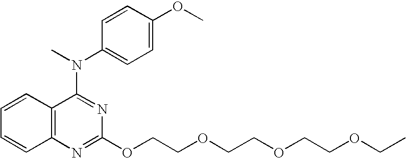

### (2-{2-[2-(2-Ethoxy-ethoxy)-ethoxy]-ethoxy}-quinazolin-4-yl)-(4-methoxy-phenyl)-methyl-amine

To a solution of (2-chloro-quinazolin-4-yl)-(4-methoxy-phenyl)-methyl-amine hydrochloride (335 mg, 1.00 mmol) in 7 mL of triethylene glycol was added 18-crown-6 (264 mg, 1.00 mmol) and potassium tert-butoxide (336 mg, 3.00 mmol). The mixture was heated at 100° C. for 1 h with stirring and then diluted with 5 mL of water and 20 mL of ethyl acetate. The organic layer was washed with water, then dried and concentrated. The resulting material was dissolved in 5 mL of DMSO, and then potassium hydroxide (168 mg, 3.00 mmol) and ethyl iodide (0.16 mL, 2.00 mmol) were added. The mixture was stirred at room temperature overnight and then diluted with 5 mL of water and 20 mL of ethyl acetate. The organic layer was washed with water, then dried and concentrated. The resulting crude material was purified by silica gel column chromatography to give the title compound (384 mg, 87%). ¹H NMR (CDCl₃) δ 7.59 (d, 1H, J=8.4 Hz), 7.45 (t, 1H, J=6.8 Hz), 7.10 (d, 2H, J=8.8 Hz), 6.92 (m, 3H), 6.83 (t, 1H, J=6.8 Hz), 4.65 (t, 2H, J=5.6 Hz), 3.94 (t, 2H, J=5.6 Hz), 3.83 (s, 3H), 3.77-3.57 (m, 8H), 3.57 (s, 3H), 3.52 (q, 2H, J=6.8 Hz), 1.20 (t, 3H, J=6.8 Hz); LC-MS (ESI⁺; 442 ([M+H]⁺).

### Example 8

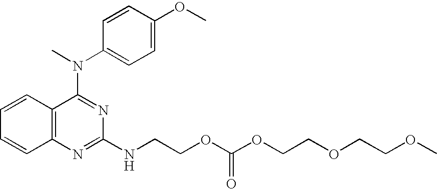

### Carbonic acid 2-(2-methoxy-ethoxy)-ethyl ester 2-{4-[(4-methoxy-phenyl)-methyl-amino]-quinazolin-2-ylamino}-ethyl ester

Carbonic acid 2,5-dioxo-pyrrolidin-1-yl ester 2-(2-methoxy-ethoxy)-ethyl ester: Carbonic acid 2-(2-methoxy-ethoxy)-ethyl ester 2-{4-[(4-methoxy-phenyl)-methyl-amino]-quinazolin-2-ylamino}-ethyl ester: A solution of (2-chloro-quinazolin-4-yl)-(4-methoxy-phenyl)-methyl-amine hydrochloride (105 mg, 0.31 mmol) in 3 mL of ethanolamine was heated at 100° C. overnight with stirring. The solution was diluted with 5 mL of water and 20 mL of ethyl acetate. The organic layer was washed with water, then dried and concentrated. The resulting material was dissolved in 3 mL of chloroform and then carbonic acid 2,5-dioxo-pyrrolidin-1-yl ester 2-(2-methoxy-ethoxy)-ethyl ester (97 mg, 0.37 mmol), triethylamine (0.065 mL, 0.47 mmol) and DMAP (4 mg, 0.031 mmol) were added. The mixture was stirred for 1 day at room temperature, and then diluted with 5 mL of water and 10 mL of methylene chloride. The organic layer was washed with water, then dried and concentrated. The resulting crude material was purified by silica gel column chromatography to give the title compound (109 mg, 75%). ¹H NMR (CDCl₃) δ 7.36 (s(br), 2H), 7.16 (d, 2H, J=8.8 Hz), 6.91 (d, 2H, J=8.8 Hz), 6.91 (d, 2H, J=8.4 Hz), 6.67 (m, 1H), 4.65 (t, 2H, J=5.6 Hz), 4.22 (m, 2H), 3.82 (s, 3H), 3.80 (m, 2H), 3.67 (m, 2H), 3.58 (m, 2H), 3.47 (m, 2H), 3.44 (s(br), 3H), 3.28 (s, 3H); LC-MS (ESI⁺; 471 ([M+H]⁺).

### Example 9

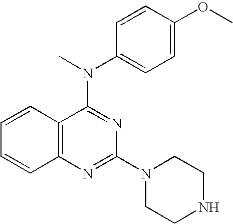

### (4-Methoxy-phenyl)-methyl-(2-piperazin-1-yl-quinazolin-4-yl)-amine

A solution of (2-chloro-quinazolin-4-yl)-(4-methoxy-phenyl)-methyl-amine hydrochloride (120 mg, 0.36 mmol) and piperazine (109 mg, 1.26 mmol) in 2 mL of 1-butanol was heated at 100° C. overnight with stirring. The mixture was concentrated and purified by silica gel column chromatography to give the title compound (107 mg, 85%). ¹H NMR (CDCl₃) δ 7.44 (d, 1H, J=8.4 Hz), 7.34 (t, 1H, J=6.8 Hz), 7.10 (d, 2H, J=8.8 Hz), 6.87 (m, 3H), 6.65 (t, 1H, J=6.8 Hz), 3.93 (t, 4H, J=5.2 Hz), 3.82 (s, 3H), 3.50 (s, 3H), 3.00 (t, 4H, J=5.2 Hz); LC-MS (ESI⁺; 350 ([M+H]⁺).

### Example 10

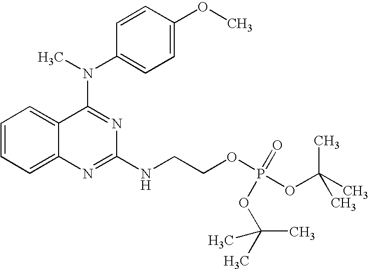

### Phosphoric acid di-tent-butyl ester 2-{4-[(4-methoxy-phenyl)-methyl-amino]-quinazolin-2-ylamino}-ethyl ester

To a solution of 2-{4-[(4-methoxy-phenyl)-methyl-amino]-quinazolin-2-ylamino}-ethanol (0.203 g, 0.62 mmol), tetrazole (0.45 M in CH₃CN; 0.36 mL) and DMAP (0.040 g, 0.3 mmol) in CH₂Cl₂ (5 mL) was added di-tent-butyl N,N-diisopropylphosphoramidite (0.347 g, 1.25 mmol), and the reaction mixture was stirred at 23° C. for 16 h. The reaction mixture was cooled to 0° C., then hydrogen peroxide (30%; 0.8 mL) was added, and the reaction stirred for another 0.5 h. The reaction mixture was diluted with CH₂Cl₂ (20 mL), and the organic layer was separated. After removal of solvents, the crude product was purified with silica gel gradient column chromatography (EtOAc/hexanes, 0% to 100%) to isolate the title compound in 85% yield with purity of 95%. A portion of the product was further purified with preparative RPLC, which gave the title compound and phosphoric acid tent-butyl ester 2-{4-[(4-methoxy-phenyl)-methyl-amino]-quinazolin-2-ylamino}-ethyl ester. ¹H NMR (CD₃OD) δ 7.38-7.34 (m, 2H), 7.11 (d, 2H, J=9.0 Hz), 6.95 (d, 2H, J=9.0 Hz), 6.88 (d, 1H, J=8.6 Hz), 6.68-6.63 (m, 1H), 4.20 (q, 2H, J=5.7 Hz), 3.81 (s, 3H), 3.77 (t, 2H, J=5.5 Hz), 3.51 (s, 3H), 1.46 (s, 9H), 1.45 (s, 9H); LC-MS (ESI⁺; 405 [M−2C₄H₈+2H+H]⁺).

### Example 11

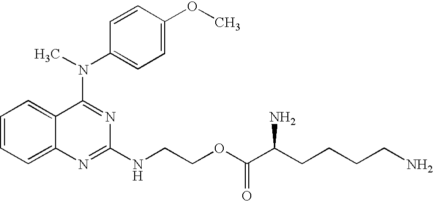

### (S)-2,6-Diamino-hexanoic acid 2-{4-[(4-methoxy-phenyl)-methyl-amino]-quinazolin-2-ylamino}-ethyl ester

A mixture of 2-{4-[(4-methoxy-phenyl)-methyl-amino]-quinazolin-2-ylamino}-ethanol (0.200 g, 0.617 mmol), Boc-L-Lys(Boc)-OH (0.277 g, 0.8 mmol), BOP-Cl (0.256 g, 1.0 mmol) and Et₃N (0.2 mL, 1.4 mmol) in CH₂Cl₂ (10 mL) was stirred at 23° C. for 16 h. Then BOP-Cl (0.256 g, 1.0 mmol) was added and the reaction mixture was stirred at 23° C. for another 4 h. The reaction mixture was diluted with EtOAc (30 mL), and the organic layer was separated, washed with satd Na₂CO₃, satd NaCl, and dried over anhydrous MgSO₄. After removal of solvents, the crude product was treated with 10% TFA solution in CH₂Cl₂, and stirred at 23° C. for 3 h. After removal of excess TFA and CH₂Cl₂, the crude product was purified with preparative RPLC to give the title compound in 45% yield as the corresponding TFA salt. ¹H NMR (CD₃OD) δ 7.62-7.58 (m, 1H), 7.44 (d, 1H, J=8.0 Hz), 7.32 (d, 2H, J=9.0 Hz), 7.09 (d, 2H, J=9.0 Hz), 6.92 (t, 1H, J=7.5 Hz), 6.76 (d, 1H, J=8.4 Hz), 4.57-4.53 (m, 2H), 4.14 (t, 1H, J=6.6 Hz), 4.10-3.86 (m, 2H), 3.87 (s, 3H), 3.72 (s, 3H), 2.94 (apparent t, 2H), 2.09-1.88 (m, 2H), 1.76-1.68 (m, 2H), 1.62-1.51 (m, 2H); LC-MS (ESI⁺; 453 ([M+H]⁺).

### Example 12

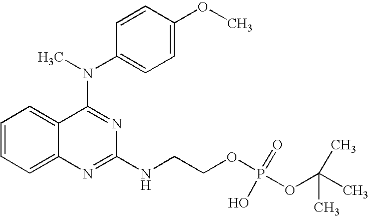

### Phosphoric acid tent-butyl ester 2-{4-[(4-methoxy-phenyl)-methyl-amino]-quinazolin-2-ylamino}-ethyl ester

The title compound was produced in the reaction of Example 10. ¹H NMR (CDCl₃) δ 10.13 (s(br), 1H), 7.43 (d, 1H, J=8.4 Hz), 7.17-7.11 (m, 3H), 6.94 (d, 2H, J=8.8 Hz), 6.58-6.54 (m, 1H), 6.49 (d, 1H, J=8.6 Hz), 4.29-4.25 (m, 2H), 3.89-3.86 (m, 2H), 3.85 (s, 3H), 3.58 (s, 3H), 1.50 (s, 9H); HRMS m/z 461.1955 [M+H]⁺.

### Example 13

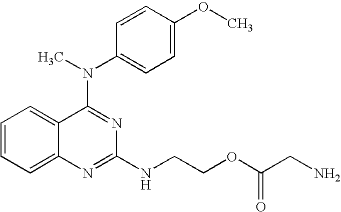

### Amino-acetic acid 2-{4-[(4-methoxy-phenyl)-methyl-amino]-quinazolin-2-ylamino}-ethyl ester

A mixture of 2-{4-[(4-methoxy-phenyl)-methyl-amino]-quinazolin-2-ylamino}-ethanol (0.196 g, 0.605 mmol), Boc-Gly-OH (0.140 g, 0.8 mmol), BOP-Cl (0.256 g, 1.0 mmol) and Et₃N (0.2 mL, 1.4 mmol) in CH₂Cl₂ (5 mL) was stirred at 23° C. for 16 h. The reaction mixture was diluted with EtOAc (20 mL) and the organic layer was separated then washed with satd Na₂CO₃, satd NaCl, and dried over anhydrous MgSO₄. After removal of solvents, the crude product was treated with 10% TFA solution in CH₂Cl₂, and stirred at 23° C. for 3 h. After removal of excess TFA and CH₂Cl₂, the crude product was purified with preparative RPLC to give the title compound as the corresponding TFA salt. ¹H NMR (CD₃OD) δ 7.64-7.60 (m, 2H), 7.45 (s, 1H), 7.34 (d, 2H, J=8.9 Hz), 7.09 (d, 2H, J=8.9 Hz), 6.95 (t, 1H, J=8.3 Hz), 6.78 (d, 1H, J=8.6 Hz), 4.46 (t, 2H, J=5.3 Hz), 4.10 (s, 2H), 3.92 (apparent t, 2H), 3.87 (s, 3H), 3.78 (apparent s, 2H), 3.72 (s, 3H); HRMS m/z: 382.1928 [M+H]⁺.

### Example 14

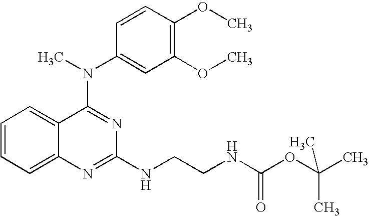

### (2-{4-[(3,4-Dimethoxy-phenyl)-methyl-amino]-quinazolin-2-ylamino}-ethyl)-carbamic acid tert-butyl ester

(3,4-Dimethoxy-phenyl)-methyl-amine: A soln of 3,4-dimethoxy-phenylamine (15.318 g, 100 mmol), ethyl-diisopropyl-amine (21.0 mL, 121 mmol) and iodomethane (7.5 mL, 120 mmol) in acetonitrile (100 mL) was heated at 40° C. for 22 h then concentrated. EtOAc (200 mL) was added and this washed with satd NaHCO₃ (1×20 mL), water (3×20 mL) and satd NaCl (2×25 mL); then dried (MgSO₄), filtered through a pad of silica and purified by MPLC (SiO₂/0-60% EtOAc in hexanes gradient) yielding a liquid (3.179 g; 19%). ¹H NMR (CDCl₃) δ 6.77 (d, J=8.4 Hz, 1H), 6.26 (d, J=2.8 Hz, 1H), 6.16 (dd, J=2.5, 8.8 Hz, 1H), 3.86 (s, 3H), 3.82 (s, 3H), 2.82 (s, 3H).

(2-Chloro-quinazolin-4-yl)-(3,4-dimethoxy-phenyl)-methyl-amine hydrochloride: Concd HCl (2 drops) was added to a mixture of (3,4-dimethoxy-phenyl)-methyl-amine (502 mg, 3.00 mmol) and 2,4-dichloro-quinazoline (88 wt %; 758 mg, 3.4 mmol) in 2-propanol (11 mL). After 16 h the insoluble reaction material was collected on a Büchner funnel, washed with 2-propanol and dried yielding the title product as a pale yellow solid (792 mg; 72%). ¹H NMR (CDCl₃) δ 8.65 (m, 1H), 7.75 (m, 1H), 7.18 (m, 1H), 7.00 (m, 1H), 6.90 (m, 1H), 6.84-6.79 (m, 2H), 3.99 (s, 3H), 3.87 (s, 3H), 3.83 (s, 3H); LC-MS (ESI⁺; 330 ([M+H]⁺).

(2-{4-[(3,4-Dimethoxy-phenyl)-methyl-amino]-quinazolin-2-ylamino}-ethyl)-carbamic acid tert-butyl ester: A mixture of (2-chloro-quinazolin-4-yl)-(3,4-dimethoxy-phenyl)-methyl-amine hydrochloride (200 mg, 0.546 mmol), ethyl-diisopropyl-amine (0.23 mL, 1.32 mmol) and (2-amino-ethyl)-carbamic acid tert-butyl ester (122 μL, 0.774 mmol) in i-PrOH (2 mL) was heated at 90° C. After 5 days, the reaction was concentrated then saturated NaHCO₃ (5 mL) was added. The product was extracted into chloroform (3×2 mL) and the combined extracts washed with water (3×2 mL) and satd NaCl (2×3 mL). This solution was dried (MgSO₄), filtered through silica and washed with 100:10:1 CHCl₃:MeOH:concentrated NH₄OH, concentrated then purified by MPLC (12 g SiO₂/0-30% (20:1 MeOH:concentrated NH₄OH) in CHCl₃ gradient). The title product was obtained as an oil, which solidified to a tan solid under high vacuum (60 mg; 24%). ¹H NMR (CDCl₃) δ 7.45-7.35 (m, 2H), 6.91-6.82 (m, 2H), 6.77-6.66 (m, 3H), 5.80 (s(br), 1H), 5.44 (s(br), 1H), 3.92 (s, 3H), 3.79 (s, 3H), 3.69 (m, 2H), 3.53 (s, 3H), 3.44 (m, 2H), 1.44 (s, 9H); LC-MS (ESI⁺; 454 ([M+H]⁺).

### Example 15

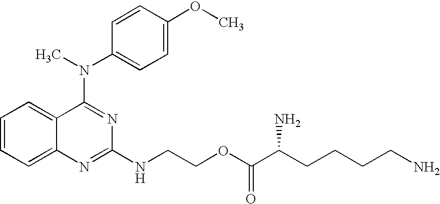

### (R)-2,6-Diamino-hexanoic acid 2-{4-[(4-methoxy-phenyl)-methyl-amino]-quinazolin-2-ylamino}-ethyl ester

A mixture of 2-{4-[(4-methoxy-phenyl)-methyl-amino]-quinazolin-2-ylamino}-ethanol (0.112 g, 0.36 mmol), Boc-D-Lys(Boc)-OH DCHA salt (0.260 g, 0.5 mmol), BOP-Cl (0.256 g, 1.0 mmol) and Et₃N (0.4 mL, 2.9 mmol) in THF (10 mL) was stirred at 23° C. for 16 h. The reaction mixture was directly purified with silica gel gradient column chromatography (EtOAc/hexanes, 0% to 100%) to give the intermediate. The intermediate was treated with 10% TFA solution in CH₂Cl₂, and stirred at 23° C. for 3 h. After removal of excess TFA and CH₂Cl₂, the crude product was purified with preparative RPLC to give the title compound in 60% yield as the corresponding TFA salt. ¹H NMR (CD₃OD) δ 7.60 (apparent t, 1H), 7.44 (d, 1H, J=8.2 Hz), 7.31 (d, 2H, J=8.2 Hz), 7.09 (d, 2H, J=7.4 Hz), 6.92 (t, 1H, J=7.8 Hz), 6.76 (d, 1H, J=8.4 Hz), 4.58-4.51 (m, 2H), 4.14 (t, 1H, J=6.5 Hz), 4.10-3.86 (m, 2H), 3.87 (s, 3H), 3.72 (s, 3H), 2.94 (apparent t, 2H), 2.09-1.92 (m, 2H), 1.76-1.68 (m, 2H), 1.62-1.51 (m, 2H); HRMS m/z 453.2627 [M+H]⁺.

### Example 16

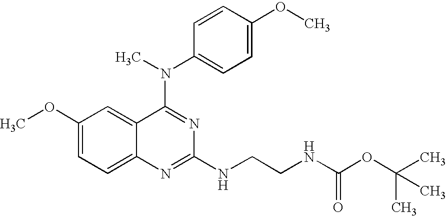

### (2-{6-Methoxy-4-[(4-methoxy-phenyl)-methyl-amino]-quinazolin-2-ylamino}-ethyl)-carbamic acid tent-butyl ester

6-Methoxy-1H-quinazoline-2,4-dione: 2-Amino-5-methoxy-benzoic acid (1.5 g, 8.98 mmol) was suspended in water (50 mL) and glacial acetic acid (0.6 mL) at 35° C. A freshly prepared solution of potassium cyanate (0.94 g, 11.67 mmol) in water (5 mL) was added dropwise to the stirred mixture. After 3 h, sodium hydroxide (12.21 g, 30.5 mmol) was added in portions, keeping the temperature below 40° C. A clear solution was obtained after a while before the precipitation of the hydrated sodium salt. After cooling, the precipitate was filtered off and dissolved in hot water which was acidified to pH 5, causing the precipitation of the title compound, which was filtered off and washed with water and dried under vacuum to give 0.85 g (50% yield). ¹H NMR (DMSO-d₆) δ 7.32 (d, 1H), 7.29-7.26 (dd, 1H), 7.12 (d, 1H), 3.78 (s, 3H).

2,4-Dichloro-6-methoxy-quinazoline: A suspension of 6-methoxy-1H-quinazoline-2,4-dione (0.85 g, 4.42 mmol) and N,N-dimethylaniline (1.66 mL, 2.6 mmol) in phosphoryl chloride (6 mL, 65.5 mmol) was heated under reflux for 5 h. The reaction mixture was cooled and poured into a beaker with ice, the precipitate was filtered off, washed with water and dried under vacuum to provide 0.82 g (82% yield). ¹H NMR (DMSO-d₆) δ 8.01 (d, 1H), 7.03-7.80 (dd, 1H), 7.5 (d, 1H), 4.0 (s, 3H).

2-Chloro-6-methoxy-quinazoline-4-yl)-(4-methoxy-phenyl)-methyl-amine: A mixture of 2,4-dichloro-6-methoxy-quinazoline (0.5 g, 2.18 mmol), (4-methoxy-phenyl)-methyl-amine (0.35 g, 2.61 mmol) and sodium acetate (0.21 g, 2.61 mmol) in 8 mL of solvent (1:1 THF:water) was stirred at 60-70° C. for 3 h. The reaction mixture was concentrated and the resulting solid was dissolved in ethyl acetate and filtered through a pad of silica, washing with 40% ethyl acetate/hexane. The filtrate was concentrated under reduced pressure to give 0.7 g of the title compound (98% yield). ¹H NMR (DMSO-d₆) δ 7.6 (d, 1H), 7.36-7.31 (m, 3H), 7.08-7.05 (dd, 2H), 6.2 (d, 1H), 3.79 (s, 3H), 3.5 (s, 3H), 3.28 (s, 3H); LC-MS (ESI⁺; 330 ([M+H]⁺).

(2-{6-Methoxy-4-[(4-methoxy-phenyl)-methyl-amino]-quinazolin-2-ylamino}-ethyl)-carbamic acid tert-butyl ester: A mixture of (2-chloro-6-methoxy-quinazoline-4-yl)-(4-methoxy-phenyl)-methyl-amine (0.6 g, 1.81 mmol), N-(2-aminomethyl)carbamic acid tert-butyl ester (0.4 g, 2.54 mmol) and ethyl-diisopropyl-amine (0.76 mL, 4.4 mmol) in 3 mL of n-BuOH was stirred at 130° C. for 2 days. The solvent was evaporated under reduced pressure. The title compound (0.56 g; 70%) was isolated by preparative TLC (CH₂Cl₂/MeOH=5/3 as eluent). ¹H NMR (DMSO-d₆) δ 7.3-7.28 (d, 1H), 7.2 (d, 2H), 7.11-7.08 (d, 1H), 7.02-7.00 (d, 2H), 6.2 (s, 1H), 3.7 (s, 3H), 3.46 (s, 3H), 3.42 (t, 2H), 3.23 (s, 3H), 3.17 (t, 2H), 1.37 (s, 9H); LC-MS (ESI⁺; 454 ([M+H]⁺).

### Example 17

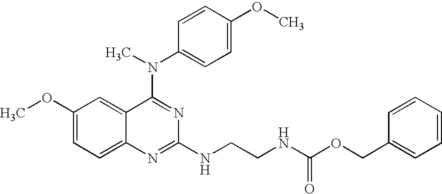

### (2-{6-Methoxy-4-[(4-methoxy-phenyl)-methyl-amino]-quinazolin-2-ylamino}-ethyl)-carbamic acid benzyl ester

A mixture of (2-chloro-6-methoxy-quinazoline-4-yl)-(4-methoxy-phenyl)-methyl-amine (0.28 g, 0.84 mmol), (2-amino-ethyl)-carbamic acid benzyl ester (0.23 g, 1.01 mmol), and triethylamine (0.6 mL, 4.3 mmol) in N-methylpyrrolidinone (3 mL) was stirred at 100° C. for 2 days. The reaction mixture was diluted with ethyl acetate, washed with sodium bicarbonate, satd NaCl, dried over MgSO₄, filtered and concentrated. Isolation of the product was done by preparative RPLC and yielded 20 mg (5%). ¹H NMR (DMSO-d₆) δ 7.5 (t, 1H), 7.4-7.3 (d, 2H), 7.32-7.30 (m, 6H), 7.12-7.10 (d, 2H), 6.11 (s, 1H), 5.02 (s, 2H), 3.81 (s, 3H), 3.61 (s, 3H), 3.9 (t, 2H), 3.31 (t, 2H), 3.25 (s, 3H); LC-MS (ESI⁺; 488 ([M+H]⁺).

### Example 18

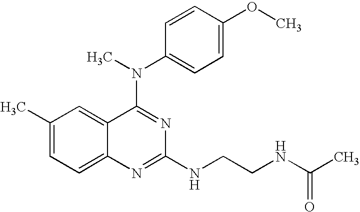

### N-(2-{4-[(4-Methoxy-phenyl)-methyl-amino]-6-methyl-quinazolin-2-ylamino}-ethyl)-acetamide

6-Methyl-1H-quinazoline-2,4-dione: A suspension of 2-amino-5-methyl-benzoic acid (4.01 g, 26.5 mmol) in H₂O (135 mL) and acetic acid (1.7 mL, 29.7 mmol) was warmed to 35° C. and then treated with KOCN (2.62 g, 32.3 mmol) in H₂O (10 mL). The resulting suspension was stirred at 35° C. for 3 h. The suspension was cooled to ±20° C. and made basic via the addition of NaOH (35.76 g, 894 mmol) at such a rate that the temperature stayed below 40° C. During the base addition the suspension momentarily went clear, then a white solid precipitated from solution. The white solid was collected via vacuum filtration and redissolved in hot H₂O (130 mL, 90° C.). HCl (1 N, 90 mL, 90 mmol) was added to this hot solution, causing a solid to precipitate. The suspension was cooled to room temperature and the solid collected via vacuum filtration. The title compound was collected as a white solid (2.35 g; 50%). ¹H NMR (DMSO-d₆) δ 11.23 (s, 1H), 11.06 (s, 1H), 7.68±7.71 (m, 1H), 7.46 (dd, 1H), 7.07 (d, 1H), 2.32 (s, 3H); GC-MS (EI) 176 (100%), 133 (95%).

2,4-Dichloro-6-methyl-quinazoline: A suspension of 6-methyl-1H-quinazoline-2,4-dione (1.20 g, 6.8 mmol) in POCl₃ (10 mL) was heated to reflux for 6 h. After cooling to room temperature the reaction was quenched by slow, dropwise addition onto ice. The resulting solid was collected via vacuum filtration, providing the title compound.

(2-Chloro-6-methyl-quinazolin-4-yl)-(4-methoxy-phenyl)-methyl-amine: A mixture of (4-methoxy-phenyl)-methyl-amine (860 mg, 6.3 mmol), NaOAc (1.52 g, 18.5 mmol), THF (34 mL), H₂O (24 mL) and the 2,4-dichloro-6-methyl-quinazoline prepared above was stirred at room temperature for 4 days. Volatile organics were then removed and the resulting solid collected via vacuum filtration. Purification by gradient MPLC (SiO₂, 0 to 50%, EtOAc/hexanes) provided the title compound as a white solid (874 mg; 44%). ¹H NMR (CDCl₃) δ 7.63 (d, 1H), 7.39 (dd, 1H), 7.05-7.18 (m, 2H), 6.92-6.98 (m, 2H), 6.62-6.66 (m, 1H), 3.86 (s, 3H), 3.61 (s, 3H), 2.09 (s, 3H).

N²-(2-Amino-ethyl)-N⁴-(4-methoxy-phenyl)-N⁴-methyl-quinazoline-2,4-diamine: A mixture of (2-chloro-6-methyl-quinazolin-4-yl)-(4-methoxy-phenyl)-methyl-amine (220 mg, 0.70 mmol) and ethane-1,2-diamine (3 mL) was heated to 100° C. for 2 h. The reaction was concentrated and purified via reverse-phase MPLC. A quantitative yield of the title compound as the trifluoroacetate salt was obtained after lyophilization. ¹H NMR (DMSO-d₆) δ 7.51 (d, 1H), 7.25-7.42 (m, 3H), 7.12 (d, 2H), 6.33 (s, 1H), 3.84 (s, 3H), 3.72-3.80 (m, 2H), 3.66 (br s, 3H), 3.08-3.18 (m, 2H), 1.96 (s, 3H); HRMS (ESI⁺) m/z calcd for C₁₉H₂₄N₅O [M+H]⁺ 338.1975, found 338.1986.

N-(2-{4-[(4-Methoxy-phenyl)-methyl-amino]-6-methyl-quinazolin-2-ylamino}-ethyl)-acetamide: Acetyl chloride (0.02 mL, 0.26 mmol) was added to a solution of N²-(2-amino-ethyl)-N⁴-(4-methoxy-phenyl)-N⁴-methyl-quinazoline-2,4-diamine trifluoroacetate (55.4 mg, 0.12 mmol) and Hünig's base (0.15 mL, 0.86 mmol) in THF (1 mL). The reaction was stirred for 24 h, concentrated directly onto Celite and purified by gradient MPLC (Isco amine column, i-PrOH/CH₂Cl₂, 0-100%) to provide 28.6 mg (56%) of the title compound. ¹H NMR (MeOH-d₄) δ 7.26 (s(br), 2H), 7.11-7.18 (m, 2H), 6.98-7.03 (m, 2H), 6.61 (s(br), 1H), 3.83 (s, 3H), 3.57-3.64 (m, 2H), 3.55 (s, 3H), 3.42-3.49 (m, 2H), 1.96-2.00 (m, 3H), 1.95 (s, 3H); LC-MS (ESI⁺; 380 ([M+H]⁺).

### Example 19

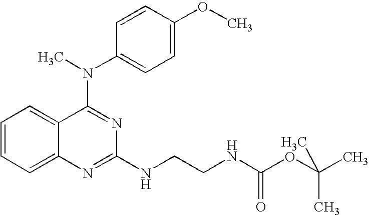

### (2-{4-[(4-Methoxy-phenyl)-methyl-amino]-quinazolin-2-ylamino}-ethyl)-carbamic acid tert-butyl ester

A mixture of (2-chloro-quinazolin-4-yl)-(4-methoxy-phenyl)-methyl-amine hydrochloride (1.50 g, 4.47 mmol) and (2-amino-ethyl)-carbamic acid tert-butyl ester (0.94 g, 5.60 mmol) with Et₃N (2 mL, 14 mmol) in isopropanol (50 mL) was stirred at 85° C. for 48 h. The reaction mixture was diluted with CH₂Cl₂ and directly charged to silica gel gradient column chromatography (MeOH/CH₂Cl₂, 0% to 10%) to obtain the title compound. ¹H NMR (CDCl₃) δ 7.44 (d, 1H, J=8.2 Hz), 7.40-7.36 (m, 1H), 7.11 (d, 2H, J=9.0 Hz), 6.91 (d, 2H, J=9.0 Hz), 6.85 (d, 1H, J=8.6 Hz), 6.72-6.67 (m, 1H), 3.84 (s, 3H), 3.68 (q, 2H, J=5.4 Hz), 3.52 (s, 3H), 3.43 (q, 2H, J=5.4 Hz), 1.43 (s, 9H); LC-MS (ESI⁺; 424 ([M+H]⁺).

### Example 20

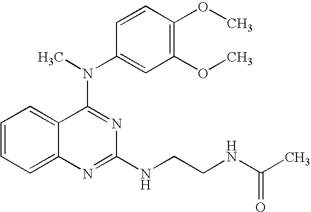

### N-(2-{4-[(3,4-Dimethoxy-phenyl)-methyl-amino]-quinazolin-2-ylamino}-ethyl)-acetamide

N²-(2-amino-ethyl)-N⁴-(3,4-dimethoxy-phenyl)-N⁴-methyl-quinazoline-2,4-diamine: A soln of (2-{4-[(3,4-dimethoxy-phenyl)-methyl-amino]-quinazolin-2-ylamino}-ethyl)-carbamic acid tert-butyl ester (550 mg, 1.21 mmol) in trifluoroacetic acid (10 mL) was stirred for 20 min, then concentrated. THF (5 mL) was added and this soln stirred over bicarbonate resin (Novabiochem 01-64-0419; 6.2 mmol g⁻¹; 0.6 g) for 1.3 h then filtered (0.45 μm) and concentrated yielding a peach-colored hard foam (412 mg; 96%). ¹H NMR (CDCl₃) δ 7.42 (m, 1H), 7.37 (m, 1H), 6.90 (m, 1H), 6.83 (m, 1H), 6.75-6.70 (m, 2H), 6.68 (m, 1H), 5.29 (s(br), 1H), 3.91 (s, 3H), 3.78 (s, 3H), 3.63 (app q, J=6.0 Hz, 2H), 3.00 (app t, J=6.0 Hz, 2H); LC-MS (ESI⁺; 354 ([M+H]⁺).

N-(2-{4-[(3,4-Dimethoxy-phenyl)-methyl-amino]-quinazolin-2-ylamino}-ethyl)-acetamide: Neat acetyl chloride (10 μL, 0.14 mmol) was added to a soln of N²-(2-amino-ethyl)-N⁴-(3,4-dimethoxy-phenyl)-N⁴-methyl-quinazoline-2,4-diamine (48 mg, 0.14 mmol) and ethyl-diisopropyl-amine (50 μL, 0.29 mmol) in DCM (5 mL). After 4 days, the soln was washed with satd NaHCO₃ (1×3 mL), dried (MgSO₄), filtered through silica and washed with 100:10:1 CHCl₃:MeOH:concd NH₄OH, concentrated then purified by MPLC (Isco amine column, 14 g (68-2203-101); 0->100% i-PrOH in CHCl₃). The title product was obtained as a pale yellow glass (32 mg, 60%). ¹H NMR (CDCl₃) δ 7.44-7.37 (m, 2H), 6.90 (m, 1H), 6.84 (m, 1H), 6.75-6.68 (m, 3H), 5.31 (t(br), 1H), 3.92 (s, 3H), 3.80 (s, 3H), 3.71 (m, 2H), 3.52 (m, 2H), 3.51 (s, 3H), 1.96 (s, 3H); LC-MS (ESI⁺; 396 ([M+H]⁺).

### Example 21

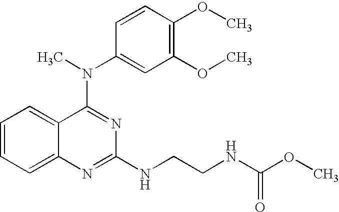

### (2-{4-[(3,4-Dimethoxy-phenyl)-methyl-amino]-quinazolin-2-ylamino}-ethyl)-carbamic acid methyl ester

This compound was prepared in an analogous manner to Example 20 using methyl chloroformate as the electrophile. Pale yellow glass (36 mg, 64%). ¹H NMR (CDCl₃) δ 7.45-7.36 (m, 2H), 6.90 (m, 1H), 6.84 (d, J=8.4 Hz, 1H), 6.76-6.67 (m, 3H), 3.92 (s, 3H), 3.79 (s, 3H), 3.70 (m, 2H), 3.67 (s, 3H), 3.52 (s, 3H), 3.49 (m, 2H); LC-MS (ESI⁺; 412 ([M+H]⁺).

### Example 22

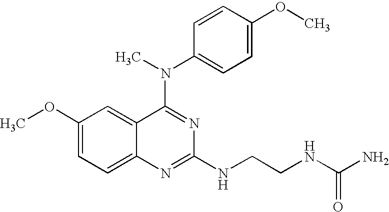

### (2-{6-Methoxy-4-[(4-methoxy-phenyl)-methyl-amino]-quinazolin-2-ylamino}-ethyl)-urea

N²-(2-Amino-ethyl)-6-methoxy-N⁴-(4-methoxy-phenyl)-N⁴-methyl-quinazoline-2,4-diamine: A solution of (2-{6-methoxy-4-[(4-methoxy-phenyl)-methyl-amino]-quinazolin-2-ylamino}-ethyl)-carbamic acid tent-butyl ester (0.56 g, 1.23 mmol) in CH₂Cl₂ (12 mL) was treated with trifluoroacetic acid (0.5 mL) at rt. After 2 h the reaction mixture was quenched by saturated aq NaHCO₃. The organics dried over MgSO₄, filtered and concentrated to afford crude product. ¹H NMR (DMSO-d₆) δ 7.45-6.13 (7H), 3.81 (s, 3H), 3.64 (s, 3H), 3.58 (m, 2H), 3.41 (m, 2H), 3.26 (s, 3H); LC-MS (ESI⁺; 397 ([M+H]⁺).

(2-{6-Methoxy-4-[(4-methoxy-phenyl)-methyl-aminc]-quinazolin-2-ylamino}-ethyl)-urea: To a solution of 90 mg (0.25 mmol) of N²-(2-amino-ethyl)-6-methoxy-N⁴-(4-methoxy-phenyl)-N⁴-methyl-quinazoline-2,4-diamine in 0.4 mL of MeOH and 1.2 mL of 1 N HCl was added solution of KOCN (70 mg in 1 mL of water), and the mixture was stirred at ambient temperature for 18 h. The solvent was evaporated, the residue dissolved in EtOAc and washed with NaHCO₃, dried over MgSO₄, filtered and concentrated. Isolation was done preparative RPLC to give 40 mg of the title compound. ¹H NMR (DMSO-d₆) δ 7.45-6.13 (7H), 3.81 (s, 3H), 3.64 (s, 3H), 3.58 (m, 2H), 3.41 (m, 2H), 3.26 (s, 3H); LC-MS (ESI⁺; 397 ([M+H]⁺).

### Example 23

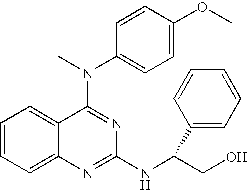

### (R)-2-{4-[(4-Methoxy-phenyl)-methyl-amino]-quinazolin-2-ylamino}-2-phenyl-ethanol

A mixture of (2-chloro-quinazolin-4-yl)-(4-methoxy-phenyl)-methyl-amine hydrochloride (0.335 g, 1.0 mmol) and (R)-2-amino-2-phenyl-ethanol (0.165 g, 1.2 mmol) with Et₃N (0.2 mL, 1.4 mmol) in isopropanol (5 mL) was stirred at 85° C. for 48 h. The reaction mixture was purified by preparative RPLC to give the title compound. ¹H NMR (DMSO-d₆) δ 12.90 (s(br), 1H), 9.00 (d, 1H, J=5.5 Hz), 7.63 (t, 1H, J=7.5 Hz), 7.35-7.46 (m, 7H), 7.27-7.31 (m, 1H), 7.06 (d, 2H, J=9.2 Hz), 6.95 (d, 1H, J=7.8 Hz), 6.58 (d, 1H, J=8.2 Hz), 5.22-5.25 (m, 1H), 3.81 (s, 3H), 3.77 (d, 2H, J=5.7 Hz), 3.50 (s, 3H); HRMS m/z 401.2764 [M+H]⁺.

### Example 24

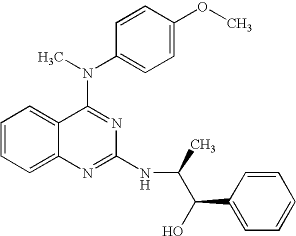

### (1R,2S)-2-{4-[(4-Methoxy-phenyl)-methyl-amino]-quinazolin-2-ylamino}-1-phenyl-propan-1-ol

A mixture of (2-chloro-quinazolin-4-yl)-(4-methoxy-phenyl)-methyl-amine hydrochloride (0.335 g, 1.0 mmol) and (1R,2S)-(−)-2-norephedrine (0.181 g, 1.2 mmol) with Et₃N (0.2 mL, 1.4 mmol) in isopropanol (5 mL) was stirred at 85° C. for 48 h. The reaction mixture was purified with preparative RPLC to give the title compound. ¹H NMR (DMSO-d₆) δ 12.83 (s(br), 1H), 8.46 (d, 1H, J=8.2 Hz), 7.63 (t, 1H, J=7.6 Hz), 7.31-7.43 (m, 6H), 7.24 (t, 1H, J=7.2 Hz), 7.10 (d, 2H, J=8.8 Hz), 6.95 (t, 1H, J=7.4 Hz), 6.62 (d, 1H, J=8.4 Hz), 5.78 (s, 1H), 4.84 (s, 1H), 4.35-4.45 (m, 1H), 3.82 (s, 3H), 3.64 (s, 3H), 1.14 (d, 3H, J=6.7 Hz); HRMS m/z: 415.2255 [M+H]⁺.

### Example 25

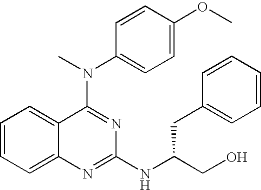

### (R)-2-{4-[(4-Methoxy-phenyl)-methyl-amino]-quinazolin-2-ylamino}-3-phenyl-propan-1-ol

A mixture of (2-chloro-quinazolin-4-yl)-(4-methoxy-phenyl)-methyl-amine hydrochloride (0.335 g, 1.0 mmol) and (R)-2-amino-3-phenyl-propan-1-ol (0.182 g, 1.2 mmol) with Et₃N (0.2 mL, 1.4 mmol) in isopropanol (5 mL) was stirred at 85° C. for 48 h. The reaction mixture was purified with preparative RPLC to give the title compound. ¹H NMR (DMSO-d₆) δ 12.84 (s(br), 1H), 8.46 (d, 1H, J=8.4 Hz), 7.61 (t, 1H, J=7.9 Hz), 7.25-7.40 (m, 6H), 7.16-7.20 (m, 1H), 7.08 (d, 2H, J=8.8 Hz), 6.94 (t, 1H, J=7.3 Hz), 6.59 (d, 1H, J=8.2 Hz), 5.12 (s, 1H), 4.38-4.46 (m, 1H), 3.82 (s, 3H), 3.58-3.64 (m, 1H), 3.57 (s, 3H), 3.34-3.48 (m, 1H), 2.84-3.04 (m, 2H); HRMS m/z: 415.3896 [M+H]⁺.

### Example 26

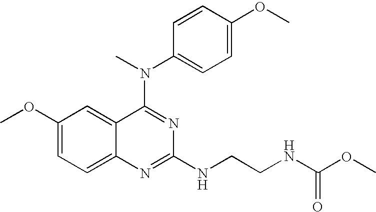

### (2-{6-Methoxy-4-[(4-methoxy-phenyl)-methyl-amino]-quinazolin-2-ylamino}-ethyl)-carbamic acid methyl ester

A mixture of N²-(2-amino-ethyl)-6-methoxy-N⁴-(4-methoxy-phenyl)-N⁴-methyl-quinazoline-2,4-diamine (0.1 g, 0.28 mmol), methyl chloroformate (29 mg, 0.31 mmol) and triethylamine (0.06 mL, 0.42 mmol) in 5 mL of DCM was stirred at ambient temperature for 1 hour. The reaction mixture was concentrated, the residue dissolved in ethyl acetate and washed with sodium bicarbonate (2×10 mL), satd NaCl, dried (MgSO₄) and filtered and the solvent was removed under reduced pressure. The crude-extract was purified by preparative TLC(CHCl₃: MeOH:concd NH₄OH=10:0.5:0.025 as eluent) and obtained 21 mg of the title compound. ¹H NMR (DMSO-d₆) δ 7.44-6.1 (7H), 3.81 (s, 3H), 3.63 (s, 3H), 3.57 (m, 2H), 3.53 (s, 3H), 3.28 (m, 2H), 3.26 (s, 3H); LC-MS (ESI⁺; 412 ([M+H]⁺).

### Example 27

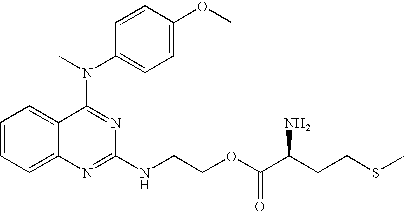

### (S)-2-Amino-4-methylsulfanyl-butyric acid 2-{4-[(4-methoxy-phenyl)-methyl-amino]-quinazolin-2-ylamino}-ethyl ester

A mixture of 2-{4-[(4-methoxy-phenyl)-methyl-amino]-quinazolin-2-ylamino}-ethanol (0.162 g, 0.5 mmol), Boc-L-Met-OH (0.249 g, 1.0 mmol), BOP-Cl (0.51 g, 2.0 mmol) and Et₃N (0.5 mL, 3.6 mmol) in THF (4 mL) was stirred at 23° C. for 16 h. The reaction mixture was directly purified with silica gel gradient column chromatography (EtOAc/hexanes, 0% to 100%) to give the intermediate. The intermediate was treated with 10% TFA solution in CH₂Cl₂, and stirred at 23° C. for 3 h. After removal of excess TFA and CH₂Cl₂, the crude product was purified with preparative RPLC to give the title compound in 60% yield as the corresponding TFA salt. ¹H NMR (CD₃OD) δ 7.59-7.63 (m, 1H), 7.44 (apparent d, 1H), 7.30-7.35 (m, 2H), 7.07-7.11 (m, 2H), 6.93 (t, 1H, J=7.8 Hz), 6.77 (d, 1H, J=8.6 Hz), 4.53-4.56 (m, 2H), 4.28 (t, 1H, J=6.3 Hz), 3.97-4.03 (m, 2H), 3.87 (s, 3H), 3.73 (s, 3H), 2.64 (t, 2H, J=7.1 Hz), 2.23-2.29 (m, 1H), 2.11-2.18 (m, 1H), 2.04 (s, 3H); HRMS m/z: 456.2108 [M+H]⁺.

### Example 28

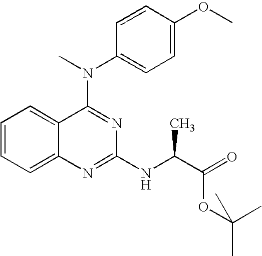

### (S)-2-{4-[(4-Methoxy-phenyl)-methyl-amino]-quinazolin-2-ylamino}-propionic acid tert-butyl ester

A mixture of (2-chloro-quinazolin-4-yl)-(4-methoxy-phenyl)-methyl-amine hydrochloride (0.200 g, 0.64 mmol) and L-alanine tert-butyl ester hydrochloride (0.182 g, 1.0 mmol) with Et₃N (0.5 mL, 3.6 mmol) in isopropanol (5 mL) was stirred at 85° C. for 48 h. The reaction mixture was diluted with CH₂Cl₂ and directly charged to silica gel gradient column chromatography (EtOAc/hexanes, 0% to 70%) to obtain the title compound. ¹H NMR (CDCl₃) δ 7.42-7.44 (m, 1H), 7.34-7.39 (m, 1H), 7.08-7.12 (m, 2H), 6.68-6.91 (m, 2H), 6.85 (d, 1H, J=8.4 Hz), 6.66-6.71 (m, 1H), 4.62-4.70 (m, 1H), 3.83 (s, 3H), 3.50 (s, 3H), 1.53 (d, 3H, J=7.0 Hz), 1.47 (s, 9H); LC-MS (ESI⁺; 409 ([M+H]⁺).

### Example 29

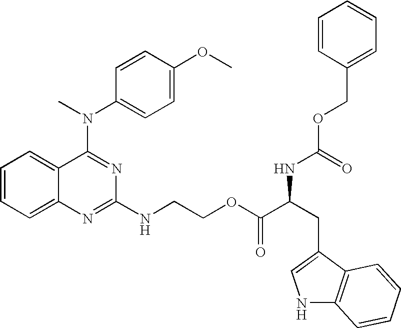

### (S)-2-Benzyloxycarbonylamino-3-(1H-indol-3-yl)-propionic acid 2-{4-[(4-methoxy-phenyl)-methyl-amino]-quinazolin-2-ylamino}-ethyl ester

A mixture of 2-{4-[(4-methoxy-phenyl)-methyl-amino]-quinazolin-2-ylamino}-ethanol (0.162 g, 0.5 mmol), Cbz-L-Trp-OH (0338 g, 1.0 mmol), BOP-Cl (0.51 g, 2.0 mmol) and Et₃N (0.5 mL, 3.6 mmol) in THF (4 mL) was stirred at 23° C. for 16 h. The reaction mixture was directly purified with silica gel gradient column chromatography (EtOAc/hexanes, 0% to 100%) to give the title compound. For analytical purposes, the crude product was further purified by preparative RPLC. ¹H NMR (CD₃OD) δ 7.55-7.59 (m, 1H), 7.34-7.42 (m, 2H), 7.23-7.27 (m, 3H), 7.13-7.20 (m, 4H), 7.03 (s(br), 2H), 6.95-7.00 (m, 2H), 6.82-6.90 (m, 2H), 6.67 (d, 1H, J=8.4 Hz), 4.90 (s, 2H), 4.83-4.93 (m, 1H), 4.47-4.51 (m, 1H), 4.36-4.42 (m, 1H), 4.25-4.31 (m, 1H), 3.83 (s, 3H), 3.75-3.80 (m, 1H), 3.59 (s, 3H), 3.23-3.28 (m, 1H), 3.07-3.13 (m, 1H); HRMS m/z 645.2826 [M+H]⁺.

### Example 30

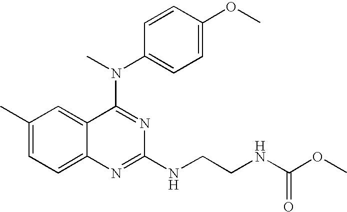

### (2-{4-[(4-Methoxy-phenyl)-methyl-amino]-6-methyl-quinazolin-2-ylamino}-ethyl)-carbamic acid methyl ester

Methyl chloroformate (0.02 mL, 0.26 mmol) was added to a solution of N²-(2-amino-ethyl)-N⁴-(4-methoxy-phenyl)-6,N⁴-dimethyl-quinazoline-2,4-diamine trifluoroacetate (50.4 mg, 0.12 mmol) and Hünig's base (0.15 mL, 0.86 mmol) in THF (1 mL). The reaction was stirred for 24 h at rt and then concentrated onto Celite and purified by gradient MPLC (amine column, i-PrOH/CH₂Cl₂, 0-100%) to provide 28.6 mg (60%) of the title compound. ¹H NMR (MeOH-d₄) δ 7.25 (s(br), 2H), 7.12-7.18 (m, 2H), 6.98-7.02 (m, 2H), 6.61 (s(br), 1H), 3.83 (s, 3H), 3.62 (s(br), 3H), 3.56-3.63 (m, 2H), 3.55 (s(br), 3H), 3.33-3.42 (m, 2H), 1.94-1.98 (m, 3H); LC-MS (ESI⁺; 396 ([M+H]⁺).

### Example 31

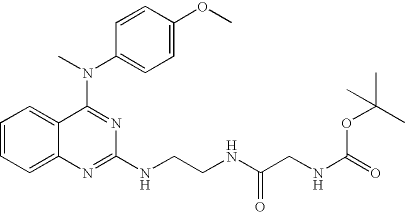

### [(2-{4-[(4-Methoxy-phenyl)-methyl-amino]-quinazolin-2-ylamino}-ethylcarbamoyl)-methyl]-carbamic acid tert-butyl ester

N²-(2-Amino-ethyl)-N⁴-(4-methoxy-phenyl)-N⁴-methyl-quinazoline-2,4-diamine: The title compound was synthesized as the TFA salt in an analogous manner to that found in Example 18. ¹H NMR (CDCl₃) δ 7.36 (m, 2H), 7.11 (m, 2H), 6.92 (m, 3H), 6.67 (m, 1H), 3.63 (t, J=7.0 Hz, 2H), 3.51 (s, 3H), 3.03 (t, J=6.8 Hz, 2H).

[(2-{4-[(4-Methoxy-phenyl)-methyl-amino]-quinazolin-2-ylamino}-ethylcarbamoyl)-methyl]-carbamic acid tert-butyl ester: A mixture of N²-(2-amino-ethyl)-N⁴-(4-methoxy-phenyl)-N⁴-methyl-quinazoline-2,4-diamine (0.150 g, 0.47 mmol), N-Boc-glycine (0.175 g, 1.0 mmol), EDCI (0.388 g, 2.0 mmol), HOBt (0.100 g, 0.74 mmol), and Et₃N (0.5 mL, 3.6 mmol) in THF 10 mL was stirred at 23° C. for 48 h. The reaction mixture was directly purified with silica gel gradient column chromatography (EtOAc/hexanes, 0% to 100%) to give the title compound. For analytical purposes, the crude product was further purified by preparative RPLC. ¹H NMR (CD₃OD) δ 7.60 (t, 1H, J=7.6 Hz), 7.37-7.34 (m, 3H), 7.05-7.10 (m, 2H), 6.89-6.94 (m, 1H), 6.77 (d, 1H, J=8.4 Hz), 3.87 (s, 3H), 3.67-3.77 (m, 7H), 3.50-3.56 (m, 2H), 1.36 (s, 9H); HRMS m/z: 481.2552 [M+H]⁺.

### Example 32

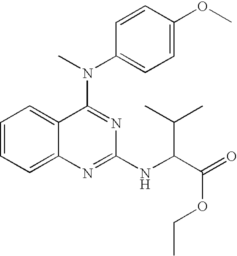

### 2-{4-[(4-Methoxy-phenyl)-methyl-amino]-quinazolin-2-ylamino}-3-methyl-butyric acid ethyl ester

A mixture of (2-chloro-quinazolin-4-yl)-(4-methoxy-phenyl)-methyl-amine hydrochloride (0.200 g, 0.64 mmol) and DL-valine ethyl ester hydrochloride (0.182 g, 1.0 mmol) with Et₃N (0.5 mL, 3.6 mmol) in isopropanol (5 mL) was stirred at 85° C. for 48 h. The reaction mixture was diluted with CH₂Cl₂ and directly charged to silica gel gradient column chromatography (EtOAc/hexanes, 0% to 70%) to obtain the title compound. For analytical purposes, the crude product was further purified by preparative RPLC. ¹H NMR (CDCl₃) δ 7.62-7.66 (m, 1H), 7.40-7.43 (m, 1H), 7.30-7.34 (m, 2H), 7.07-7.11 (m, 2H), 6.94-6.98 (m, 1H), 6.77 (d, 1H, J=8.2 Hz), 4.66 (d, 1H, J=5.7 Hz), 4.23-4.32 (m, 2H), 3.87 (s, 3H), 3.68 (s, 3H), 2.34-2.42 (m, 1H), 1.31 (t, 3H, J=7.2 Hz), 1.13 (d, 3H, J=3.5 Hz), 1.11 (d, 3H, J=3.5 Hz); HRMS m/z 409.2223 [M+H]⁺.

### Example 33

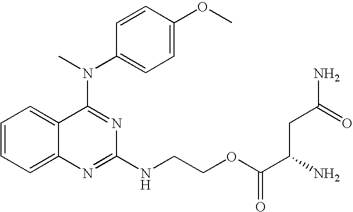

### (S)-2-Amino-succinamic acid 2-{4-[(4-methoxy-phenyl)-methyl-amino]-quinazolin-2-ylamino}-ethyl ester

A mixture of 2-{4-[(4-methoxy-phenyl)-methyl-amino]-quinazolin-2-ylamino}-ethanol (0.162 g, 0.5 mmol), Boc-L-Asn-OH (0.232 g, 1.0 mmol), BOP-Cl (0.51 g, 2.0 mmol) and Et₃N (0.2 mL, 1.4 mmol) in THF (3 mL) was stirred at 23° C. for 24 h. The reaction mixture was directly charged to silica gel gradient column chromatography (methanol/dichloromethane, 0% to 10%) to obtain the intermediate. The intermediate was treated with 10% TFA solution in CH₂Cl₂, and stirred at 23° C. for 3 h. After removal of excess TFA and CH₂Cl₂, the crude product was purified with preparative RPLC to give the title compound as the corresponding TFA salt. ¹H NMR (CD₃OD) δ 7.59-7.63 (m, 1H), 7.44 (apparent d, 1H), 7.30-7.33 (m, 2H), 7.07-7.11 (m, 2H), 6.93 (apparent t, 1H), 6.78 (d, 1H, J=8.6 Hz), 4.49-4.58 (m, 2H), 4.31-4.34 (m, 1H), 3.94-3.99 (m, 2H), 3.87 (s, 3H), 3.72 (s, 3H), 2.98 (dd, 1H, J=17.0, 4.3 Hz), 2.86 (dd, 1H, J=17.0, 4.3 Hz); LC-MS (ESI⁺; 439 ([M+H]⁺).

### Example 34

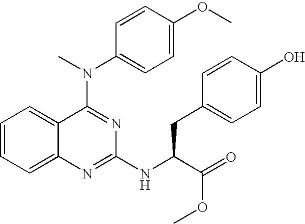

### (S)-3-(4-Hydroxy-phenyl)-2-{4-[(4-methoxy-phenyl)-methyl-amino]-quinazolin-2-ylamino}-propionic acid methyl ester

A mixture of (2-chloro-quinazolin-4-yl)-(4-methoxy-phenyl)-methyl-amine hydrochloride (0.200 g, 0.64 mmol) and L-tyrosine methyl ester hydrochloride (0.195 g, 1.0 mmol) with Et₃N (0.5 mL, 3.6 mmol) in isopropanol (5 mL) was stirred at 85° C. for 48 h. Then, the reaction mixture was further heated at 130° C. for 20 min under microwave irradiation. The reaction mixture was purified by preparative RPLC to give the title compound. ¹H NMR (CDCl₃) δ 7.76-7.80 (m, 1H), 7.61-7.64 (m, 1H), 7.44 (apparent s, 4H), 7.30-7.34 (m, 2H), 7.09-7.15 (m, 3H), 6.86 (d, 1H, J=8.0 Hz), 4.38 (t, 1H, J=6.8 Hz), 3.87 (s, 3H), 3.80 (s, 3H), 3.45 (s, 3H), 3.28-3.31 (m, 2H); LC-MS (ESI⁺; 459 ([M+H]⁺).

### Example 35

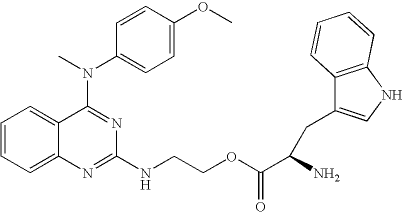

### (R)-2-Amino-3-(1H-indol-3-yl)-propionic acid 2-{4-[(4-methoxy-phenyl)-methyl-amino]-quinazolin-2-ylamino}-ethyl ester

A mixture of 2-{4-[(4-methoxy-phenyl)-methyl-amino]-quinazolin-2-ylamino}-ethanol (0.100 g, 0.3 mmol), Boc-D-Trp-OH (0.150 g, 0.5 mmol), BOP-Cl (0.256 g, 1.0 mmol) and Et₃N (0.2 mL, 1.4 mmol) in CH₂Cl₂ (5 mL) was stirred at 23° C. for 16 h. The reaction mixture was diluted with EtOAc (20 mL), and the organic layer was separated washed with satd Na₂CO₃, satd NaCl, and dried over anhydrous MgSO₄. After removal of solvents, the crude product was treated with 10% TFA solution in CH₂Cl₂, and stirred at 23° C. for 3 h. After removal of excess TFA and CH₂Cl₂, the crude product was purified with preparative RPLC to give the title compound as the corresponding TFA salt. ¹H NMR (CD₃OD) δ 7.60-7.64 (m, 2H), 7.43 (d, 1H, J=7.2 Hz), 7.12-7.23 (m, 4H), 7.05 (d, 2H, J=8.2 Hz), 6.87-6.95 (m, 3H), 6.73 (d, 1H, J=8.6 Hz), 4.38-4.44 (m, 3H), 3.86 (s, 3H), 3.76-3.80 (m, 2H), 3.66 (s, 3H), 3.43 (d, 2H, J=5.5 Hz); LC-MS (ESI⁺; 511 ([M+H]⁺).

### Example 36

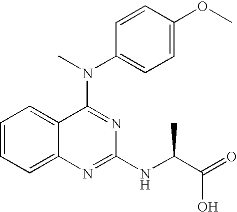

### (S)-2-{4-[(4-Methoxy-phenyl)-methyl-amino]-quinazolin-2-ylamino}-propionic acid

A mixture of (S)-2-{4-[(4-methoxy-phenyl)-methyl-amino]-quinazolin-2-ylamino}-propionic acid tent-butyl ester (0.100 g, 0.24 mmol) with potassium tert-butoxide (0.112 g) in THF (5 mL) and water (1 mL) was stirred at 23° C. for 16 h. Then, it was stirred at reflux for another 4 h. The reaction mixture was purified with preparative RPLC to obtain the title compound. ¹H NMR (CD₃OD) δ 7.60-7.64 (m, 1H), 7.43 (d, 1H, J=8.2 Hz), 7.29-7.32 (m, 2H), 7.06-7.10 (m, 2H), 6.91-6.96 (m, 1H), 6.75 (d, 1H, J=8.6 Hz), 4.71 (q, 1H, J=7.2 Hz), 3.87 (s, 3H), 3.67 (s, 3H), 1.64 (d, 3H, J=7.2 Hz); LC-MS (ESI⁺; 353 ([M+H]⁺).

### Example 37

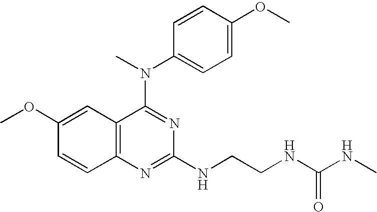

### 1-(2-{6-Methoxy-4-[(4-methoxy-phenyl)-methyl-amino]-quinazolin-2-ylamino}-ethyl)-3-methyl-urea

A mixture of N²-(2-amino-ethyl)-6-methoxy-N⁴-(4-methoxy-phenyl)-N⁴-methyl-quinazoline-2,4-diamine (0.13 g, 0.36 mmol) and N-succinimidyl-N-methylcarbamate (82 mg, 0.47 mmol) in DCM (5 mL) was stirred at ambient temperature for 3 hours. The precipitate was filtered off. The filtrate was concentrated and the crude extract was purified by preparative RPLC. ¹H NMR (DMSO-d₆) δ 7.45-6.02 (m, 8H), 3.81 (s, 3H), 3.63 (s, 3H), 3.55 (m, 2H), 3.27 (m, 2H), 3.26 (s, 3H); LC-MS (ESI⁺; 411 ([M+H]⁺).

### Example 38

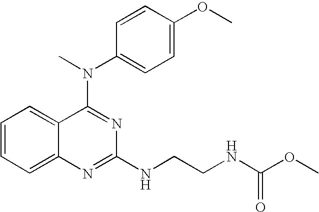

### (2-{4-[(4-Methoxy-phenyl)-methyl-amino]-quinazolin-2-ylamino}-ethyl)-carbamic acid methyl ester

To a solution of N²-(2-amino-ethyl)-N⁴-(4-methoxy-phenyl)-N⁴-methyl-quinazoline-2,4-diamine (275 mg, 0.5 mmol) and methyl chloroformate (48 mg, 0.5 mmol) in dichloromethane (2 mL) was added triethylamine (277 μL, 2 mmol). After stirring at room temperature overnight, the reaction was concentrated then purified by preparative RPLC to give 11 mg (6%). ¹H NMR (CDCl₃) δ 10.12 (s, 1H), 7.52-7.46 (m, 2H), 7.19-7.16 (m, 2H), 7.02-6.99 (m, 2H), 6.84-6.79 (m, 2H), 6.65 (d, 1H), 3.89 (s, 3H), 3.72-3.67 (m, 8H), 3.56-3.51 (m, 2H); LC-MS (ESI⁺; 382 ([M+H]⁺).

### Example 39

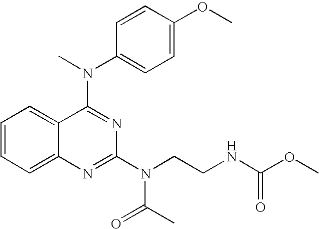

### N-[2-(Acetyl-{4-[(4-methoxy-phenyl)-methyl-amino]-quinazolin-2-yl}-amino)-ethyl]-acetamide

To a solution of N²-(2-amino-ethyl)-N⁴-(4-methoxy-phenyl)-N⁴-methyl-quinazoline-2,4-diamine (275 mg, 0.5 mmol) and acetic anhydride (51 mg, 0.5 mmol) in dichloromethane (4 mL) was added triethylamine (277 μL, 2 mmol). The reaction stirred at 100° C. for 20 minutes then stirred at room temperature overnight. Evaporated and purified by preparative RPLC to give 40 mg (20%) of the title compound. ¹H NMR (CDCl₃) δ 7.67-7.54 (m, 2H), 7.43 (bs, 1H), 7.18-7.15 (m, 2H), 7.01-6.94 (m, 4H), 4.32-4.29 (m, 2H), 3.87 (s, 3H), 3.61-3.58 (m, 5H), 2.51 (s, 3H), 1.97 (s, 3H); LC-MS (ESI⁺; 408 ([M+H]⁺).

### Example 40

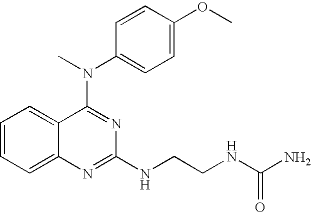

### (2-{4-[(4-Methoxy-phenyl)-methyl-amino]-quinazolin-2-ylamino}-ethyl)-urea

To a solution of N²-(2-amino-ethyl)-N⁴-(4-methoxy-phenyl)-N⁴-methyl-quinazoline-2,4-diamine (137 mg, 0.25 mmol), methanol (0.4 mL) and 1 N aqueous HCl (1.2 mL) was added potassium cyanate (70 mg, 0.75 mmol) in water (1 mL). The reaction stirred overnight at room temperature. Evaporated the MeOH and neutralized the aqueous mixture. Extracted with dichloromethane and concentrated. Purified by preparative RPLC to give 4 mg (4%) of the title compound. ¹H NMR (CDCl₃) δ 10.12 (s, 1H), 7.52-7.46 (m, 2H), 7.19-7.16 (m, 2H), 7.01-6.99 (m, 2H), 6.82-6.80 (m, 2H), 6.65 (d, 1H), 3.89 (s, 3H), 3.74-3.69 (m, 8H), 3.58-3.53 (m, 2H); LC-MS (ESI⁺; 367 ([M+H]⁺).

### Example 41

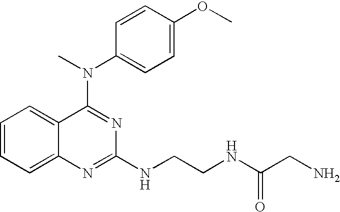

### 2-Amino-N-(2-{4-[(4-methoxy-phenyl)-methyl-amino]-quinazolin-2-ylamino}-ethyl)-acetamide

A mixture of 2-{4-[(4-methoxy-phenyl)-methyl-amino]-quinazolin-2-ylamino}-ethanol (0.196 g, 0.605 mmol), Boc-Gly-OH (0.140 g, 0.8 mmol), BOP-Cl (0.256 g, 1.0 mmol) and Et₃N (0.2 mL, 1.4 mmol) in CH₂Cl₂ (5 mL) was stirred at 23° C. for 16 h. The reaction mixture was diluted with EtOAc (20 mL), and the organic layer was separated, washed with satd Na₂CO₃, satd NaCl, and dried over anhydrous MgSO₄. After removal of solvents, the crude product was treated with 10% TFA solution in CH₂Cl₂, and stirred at 23° C. for 3 h. After removal of excess TFA and CH₂Cl₂, the crude product was purified with preparative RPLC to give the title compound as the corresponding TFA salt. ¹H NMR (CD₃OD) δ 7.60 (m, 1H), 7.41 (m, 1H), 7.32 (m, 2H), 7.09 (m, 2H), 6.91 (m, 1H), 6.76 (m, 1H), 3.87 (s, 3H), 3.78-3.66 (m, 7H), 3.60 (m, 2H); LC-MS (ESI⁺; 381 ([M+H]⁺).

### Example 42

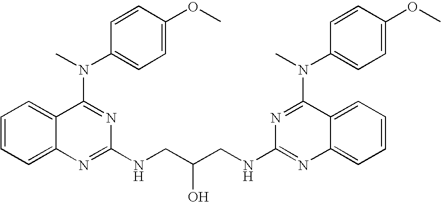

### 1,3-Bis-{4-[(4-methoxy-phenyl)-methyl-amino]-quinazolin-2-ylamino}-propan-2-ol

A mixture of (2-chloro-quinazolin-4-yl)-(4-methoxy-phenyl)-methyl-amine hydrochloride (203 mg, 0.604 mmol), ethyl-diisopropyl-amine (0.25 mL, 1.4 mmol) and 1,3-diamino-propan-2-ol (95%; 140 mg, 1.5 mmol) in n-butanol (4 mL) was heated at 115° C. After 32 h, the reaction was concentrated then purified by RPLC yielding the TFA salt. Free base was obtained by dissolving the product in EtOAc, washing with aq bicarbonate, drying (MgSO₄), filtering and concentrated yielding the title compound as a pale yellow oil (9 mg, 2%). ¹H NMR (CDCl₃) δ 7.49 (m, 2H), 7.37 (m, 2H), 7.11 (m, 4H), 6.92-6.84 (m, 6H), 6.68 (m, 2H), 5.81 (s(br), 2H), 4.11 (m, 1H), 3.91 (m, 2H), 3.83 (s, 6H), 3.61 (m, 2H), 3.51 (s, 6H); LC-MS (ESI⁺; 617 ([M+H]⁺).

### Example 43

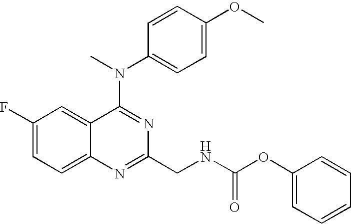

### {6-Fluoro-4-[(4-methoxy-phenyl)-methyl-amino]-quinazolin-2-ylmethyl}-carbamic acid phenyl ester

2-Chloromethyl-5-fluoro-3H-quinazolin-4-one: The title compound was prepared in a manner similar to that of 2-chloromethyl-3H-quinazolin-4-one in Example 45. ¹H NMR (DMSO-d₆) δ 12.75 (s(br), 1H), 7.65-7.85 (m, 3H), 4.55 (s, 2H).

4-Chloro-2-chloromethyl-6-fluoro-quinazoline: A suspension of 2-chloromethyl-5-fluoro-3H-quinazolin-4-one (0.836 g, 3.9 mmol) and NEt₃ (0.60 mL, 101.19 mL) in CHCl₃ (20 mL) was treated with POCl₃ and heated at reflux overnight. The reaction was quenched by pouring onto ice and extracted with CH₂Cl₂. The organics were dried (MgSO₄), filtered and concentrated. The residue was purified by gradient MPLC (SiO₂, EtOAc/hexanes, 0-100%) to provide 265 mg (30%) of the title compound. ¹H NMR (DMSO-d₆) δ 8.23 (ddd, 1H), 8.15-8.06 (m, 2H), 4.97 (s, 2H); LC-MS (ESI⁺; 231 ([M+H]⁺).

(2-Chloromethyl-6-fluoro-quinazolin-4-yl)-(4-methoxy-phenyl)-methyl-amine: A suspension of 4-chloro-2-chloromethyl-5-fluoroquinazoline (265 mg, 1.15 mmol) and (4-methoxy-phenyl)-methyl-amine (175 mg, 1.26 mmol) in i-PrOH (10 mL) was treated with concd HCl (4 drops) and stirred overnight at rt. The reaction was diluted with CH₂Cl₂ and washed with 5% NaOH. The organics were dried (MgSO₄), concentrated and purified by gradient MPLC (SiO₂, EtOAc/hexanes, 0-100%, 20 min) to provide 258 mg (68%) of the title compound. ¹H NMR (DMSO-d₆) δ 7.83 (dd, 1H), 7.62 (ddd, 1H), 7.25-7.33 (m, 2H), 7.02-7.10 (m, 2H), 6.47 (dd, 1H), 4.75 (s, 2H), 3.81 (s, 3H), 3.54 (s, 3H); LC-MS (ESI⁺; 332 ([M+H]⁺).

(2-Aminomethyl-6-fluoro-quinazolin-4-yl)-(4-methoxy-phenyl)-methyl-amine: A suspension of (2-chloromethyl-5-fluoro-quinazolin-4-yl)-(4-methoxy-phenyl)-methyl-amine (258 mg, 0.79 mL) in DMF (3 mL) was treated with potassium phthalimide (316 mg, 1.71 mmol). The reaction was heated to 70° C. for 10 min, cooled to rt, diluted with EtOAc, washed with H₂O, dried (MgSO₄) filtered and concentrated. The residue was suspended in EtOH, treated with excess hydrazine mono-hydrate and stirred overnight. The reaction was diluted with EtOAc and H₂O and the layers separated. The aqueous layer was extracted with EtOAc. The combined organics were dried (MgSO₄), filtered, concentrated and purified by gradient MPLC (SiO₂, MeOH/CH₂Cl₂ with 0.1% NH₄OH, 0-20%) to provide the title compound. ¹H NMR (CDCl₃) δ 7.77 (dd, 1H), 7.27-7.37 (m, 1H), 7.08-7.15 (m, 2H), 6.90-6.97 (m, 2H), 6.60 (dd, 1H), 4.06 (s, 2H), 3.86 (s, 3H), 3.59 (s, 3H), 1.80 (s(br), 2H); HRMS (ES) calcd for C₁₇H₁₈FN₄O (M+H) 313.1459, found 313.1502.

(2-Aminomethyl-6-fluoro-quinazolin-4-yl)-(4-methoxy-phenyl)-methyl-amine bis(hydrochloride): A solution of (2-aminomethyl-6-fluoro-quinazolin-4-yl)-(4-methoxy-phenyl)-methyl-amine was in MeOH was treated with excess 1.0 N HCl in Et₂O, Solvent was removed, yielding 131.3 mg (43%, 2 steps) of the title compound. ¹H NMR (DMSO-d₆) δ 8.43 (s(br), 3H), 7.82 (dd, 1H), 7.65 (td, 1H), 7.25-7.32 (m, 2H), 7.04-7.10 (m, 2H), 6.47 (dd, 1H), 4.23 (br q, 2H), 3.82 (s, 3H), 3.58 (s, 3H).

{6-Fluoro-4-[(4-methoxy-phenyl)-methyl-amino]-quinazolin-2-ylmethyl}-carbamic acid phenyl ester: A suspension of (2-aminomethyl-6-fluoro-quinazolin-4-yl)-(4-methoxy-phenyl)-methyl-amine bis(hydrochloride) (39.5 mg, 0.10 mmol) and NEt₃ (0.05 mL, 0.36 mmol) in CH₂Cl₂ (1 mL) was treated with phenyl chloroformate (0.02 mL, 0.16 mmol) and stirred overnight at rt. The reaction was concentrated and purified by RP-MPLC (MeCN/H₂O w/0.1% TFA) to obtain the title compound. ¹H NMR (CDCl₃) δ 8.24 (s(br), 2H), 7.52 (br t, 1H), 7.28-7.38 (m, 2H), 7.11-7.24 (m, 5H), 7.04-7.10 (m, 2H), 6.36 (d, 1H), 4.81 (s(br), 2H), 3.91 (s, 3H), 3.80 (s, 3H); LC-MS (ESI⁺; 433 ([M+H]⁺).

### Example 44

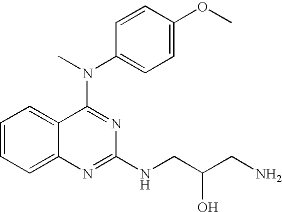

### 1-Amino-3-{4-[(4-methoxy-phenyl)-methyl-amino]-quinazolin-2-ylamino}-propan-2-ol

The title compound was produced in the reaction of Example 42. Free base was obtained as a tan solid (17 mg, 8%) after treating a soln of the TFA salt with bicarbonate resin (Novabiochem 01-64-0419) then filtration and concentrated. ¹H NMR (CDCl₃) δ 7.43-7.34 (m, 2H), 7.10 (m, 2H), 6.90 (m, 2H), 6.86 (m, 1H), 6.69 (m, 1H), 5.32 (t(br), 1H), 3.83 (s, 3H), 3.80 (m, 1H), 3.66 (m, 1H), 3.59 (m, 1H), 3.48 (s, 3H), 2.83 (m, 2H); LC-MS (ESI⁺; 354 ([M+H]⁺).

### Example 45

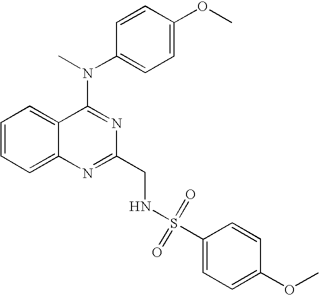

### 4-Methoxy-N-{4-[(4-methoxy-phenyl)-methyl-amino]-quinazolin-2-ylmethyl}-benzenesulfonamide

2-Chloromethyl-3H-quinazolin-4-one: A solution of methyl anthranilate (10.0 mL, 77.3 mmol) and chloroacetonitrile (5.5 mL, 87.1 mmol) in dioxane (150 mL) was treated with concd HCl (10 mL, 120 mmol) and the resulting suspension refluxed overnight. The suspension was cooled to rt and the solid collected via vacuum filtration and washed with hexanes. The solid thus obtained was suspended in H₂O and neutralized with NaHCO₃. The solid was collected via vacuum filtration and dried under vacuum to yield 8.864 g (59%) of the title compound as a white solid. ¹H NMR (DMSO-d₆) δ 12.6 (s(br), 1H), 8.13 (ddd, 1H), 7.85 (ddd, 1H), 7.69 (ddd, 1H), 7.56 (ddd, 1H), 4.56 (s, 2H); LC-MS (ESI⁺; 195 ([M+H]⁺).

4-Chloro-2-chloromethyl-quinazoline: A suspension of 2-chloromethyl-3H-quinazolin-4-one (12.27 g) in toluene (200 mL) was treated with Hünig's base (19 mL, 109 mmol) and POCl₃ (8.8 mL, 96.1 mmol) and heated to 65° C. overnight. The reaction was cooled to rt and the layers separated. The bottom layer was extracted with toluene. The top layers were combined and washed with cold H₂O and satd NaHCO₃, dried (MgSO₄), filtered and concentrated. Purification by gradient MPLC (SiO₂, 120 g column, EtOAc/hexanes, 0-100%) provided 9.72 g (69%) of the title compound as a white solid. ¹H NMR (DMSO-d₆) δ 8.33 (ddd, 1H), 8.05-8.22 (m, 2H), 7.93 (ddd, 1H), 4.97 (s, 2H); LC-MS (ESI⁺; 213 ([M+H]⁺).

(2-Chloromethyl-quinazolin-4-yl)-(4-methoxy-phenyl)-methyl-amine hydrochloride: A suspension of 4-chloro-2-chloromethyl-quinazoline (7.383 g, 35.0 mmol) and (4-methoxy-phenyl)-methyl-amine (4.837 g, 35.3 mmol) in i-PrOH (50 mL) was treated with concd HCl (1.5 mL, 18 mmol) and stirred at rt for 2 h. The resulting solid was collected by vacuum filtration, yielding 10.367 g (85%) of the title compound. ¹H NMR (DMSO-d₆) δ 7.80-7.94 (m, 2H), 7.40-7.80 (m, 2H), 7.26-7.34 (m, 1H), 7.07-7.15 (m, 2H), 6.83 (br d, 1H), 4.94 (s, 2H), 3.83 (s, 3H), 3.72 (s, 3H); LC-MS (ESI⁺; 314 ([M+H]⁺).

2-{4-[(4-Methoxy-phenyl)-methyl-amino]-quinazolin-2-ylmethyl}-isoindole-1,3-dione: A suspension of (2-chloromethyl-quinazolin-4-yl)-(4-methoxy-phenyl)-methyl-amine hydrochloride (10.367 g, 16.2 mmol) and K₂CO₃ (2.25 g, 16.3 mmol) in DMF (50 mL) was heated to 70° C. for 1 h. The reaction was cooled to rt, potassium phthalimide (6.004 g, 32.5 mmol) was added and the reaction heated to 70° C. for 2 h. The reaction was cooled to rt, diluted with EtOAc, washed with H₂O and 5% NaOH, dried (MgSO₄), filtered and concentrated. The residue was purified by gradient MPLC (SiO₂, EtOAc/hexanes 0-100%) to yield 8.56 g (68%) of the title compound. ¹H NMR (DMSO-d₆) δ 7.95-8.02 (m, 2H), 7.87-7.94 (m, 2H), 7.55-7.60 (m, 2H), 7.18-7.22 (m, 2H), 7.02-7.12 (m, 1H), 6.94-7.01 (m, 2H), 6.88 (dt, 1H), 4.95 (s, 2H), 3.77 (s, 3H), 3.26 (s, 3H); HRMS (ES) calcd for C₂₅H₂₁N₄O₃ (M+H) 425.1608, found 425.1604.

(2-Aminomethyl-quinazolin-4-yl)-(4-methoxy-phenyl)-methyl-amine: A solution of 2-{4-[(4-methoxy-phenyl)-methyl-amino]-quinazolin-2-ylmethyl}-isoindole-1,3-dione (8.561 g, 20.2 mmol) in EtOH (100 mL) was treated with hydrazine mono-hydrate (3.0 mL, 61.8 mmol) and heated to 60° C. for 2 h. The reaction was cooled to rt, HCl (2 N, 50 mL) added and the reaction heated to 60° C. for 30 min. After cooling to rt the solid was filtered off. The filtrate was concentrated, basified with 5% NaOH and extracted with CH₂Cl₂. The organic layers were combined, dried (MgSO₄), filtered and concentrated. The residue was purified by gradient reverse phase MPLC (MeCN/H₂O with 0.1% TFA) and the free base re-extracted as above to yield 3.10 g (52%) of the title compound. ¹H NMR (CDCl₃) δ 7.76 (d, 1H), 7.54 (ddd, 1H), 7.08-7.16 (m, 2H), 6.95-7.05 (m, 2H), 6.86-6.94 (m, 2H), 4.07 (s, 2H), 3.84 (s, 3H), 3.60 (s, 3H), 2.00 (s(br), 2H). ¹³C NMR (CDCl₃) δ 165.9, 162.0, 158.2, 152.2, 141.6, 132.0, 128.2, 127.6, 126.5, 124.4, 115.5, 115.4, 55.7, 49.1, 43.0; HRMS (ES) calcd for C₁₇H₁₈N₄O (M+H) 295.1553, found 295.1506.

(2-Aminomethyl-quinazolin-4-yl)-(4-methoxy-phenyl)-methyl-amine bis(hydrochloride): The corresponding free base was suspended in H₂O and treated with 2 N HCl. The resulting solution was lyophilized, providing the title compound. ¹H NMR (MeOH-d₄) δ 7.88 (ddd, 1H), 7.74 (ddd, 1H), 7.36-7.42 (m, 2H), 7.30 (ddd, 1H), 7.12-7.19 (m, 2H), 6.95 (d, 1H), 4.56 (s, 2H), 3.90 (s, 6H). Anal. calcd for C₁₇H₁₇N₄O.2HCl.1.94H₂O: C, 50.76; H, 5.98; N, 13.93; Cl, 17.63. Found: C, 50.76; H, 5.98; N, 13.93; Cl, 18.03.

(2-Aminomethyl-quinazolin-4-yl)-(4-methoxy-phenyl)-methyl-amine hydrogen sulfate: The corresponding free base (1.100 g, 3.74 mmol) in i-PrOH (4 mL) was added to a solution of H₂SO₄ (0.20 mL, 3.76 mmol) in H₂O (1.3 mL) and the mixture stirred for 10 min. Solvent was removed and the residue recrystallized from 3:1 i-PrOH:H₂O to provide the title compound. ¹H NMR (DMSO-d₆) δ 7.75 (dd, 1H), 7.69 (ddd, 1H), 7.22-7.27 (m, 2H), 7.16 (ddd, 1H), 7.02-7.08 (m, 2H), 6.97 (dd, 1H), 4.24 (s, 2H), 3.80 (s, 3H), 3.57 (s, 3H). ¹³C NMR (DMSO-d₆) δ 161.6, 158.7, 158.6, 151.6, 140.8, 133.4, 128.3, 128.1, 126.7, 125.8, 116.1, 115.4, 56.1, 44.1, 43.4. Anal. calcd for C₁₇H₁₇N₄O.H₂SO₄.0.52H₂O: C, 50.82; H, 5.28; N, 13.94; S, 7.98. Found: C, 50.82; H, 5.21; N, 13.89; S, 8.04.

4-Methoxy-N-{4-[(4-methoxy-phenyl)-methyl-amino]-quinazolin-2-ylmethyl}-benzenesulfonamide: A solution of (2-aminomethyl-quinazolin-4-yl)-(4-methoxy-phenyl)-methyl-amine (100 mg, 0.34 mmol), NEt₃ (0.11 mL, 0.79 mmol) and 4-methoxybenzenesulfonyl chloride (77 mg, 0.37 mmol) in CH₂Cl₂ (2 mL) was stirred overnight at rt. The reaction was concentrated onto SiO₂ and purified by gradient MPLC (SiO₂, EtOAc/hexanes, 0-100%) to provide 69 mg (44%) of the title compound. ¹H NMR (CDCl₃) δ 7.82-7.87 (m, 2H), 7.67 (d, 1H), 7.54 (ddd, 1H), 7.04-7.10 (m, 2H), 6.95-7.02 (m, 1H), 6.87-6.95 (m, 3H), 6.81-6.87 (m, 2H), 6.14 (t, 1H), 4.34 (d, 2H), 3.85 (s, 3H), 3.75 (s, 3H), 3.51 (s, 3H); HRMS (ES) calcd for C₂₄H₂₄N₄O₄S (M+H) 465.1591, found 465.1592.

### Example 46

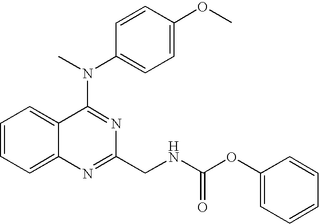

### {4-[(4-Methoxy-phenyl)-methyl-amino]-quinazolin-2-ylmethyl}-carbamic acid phenyl ester

Neat phenyl chloroformate (75 μL, 0.60 mmol) was added to a turbid soln of (2-aminomethyl-quinazolin-4-yl)-(4-methoxy-phenyl)-methyl-amine sulfate salt (157 mg, 0.400 mmol) and triethylamine (0.20 mL, 1.4 mmol) in DCM (4 mL). After overnight, the reaction was concentrated and EtOAc (8 mL) was added. This was washed with satd NaHCO₃ (1×2 mL), water (3×2 mL) and satd NaCl (2×3 mL); then dried (MgSO₄) and filtered through a plug of silica with an EtOAc wash. The title compound was obtained as an off-white solid (136 mg, 82%) after purification by MPLC (12 g SiO₂; eluting with a 0->55% gradient of EtOAc in hexanes). ¹H NMR (CDCl₃) δ 7.78 (m, 1H), 7.57 (m, 1H), 7.38 (m, 2H), 7.24-7.18 (m, 3H), 7.14 (m, 2H), 7.03-7.01 (m, 2H), 6.93 (m, 2H), 6.59 (t(br), 1H), 4.69 (d, J=4.8 Hz, 2H), 3.85 (s, 3H), 3.60 (s, 3H); LC-MS (ESI⁺; 415 ([M+H]⁺).

### Example 47

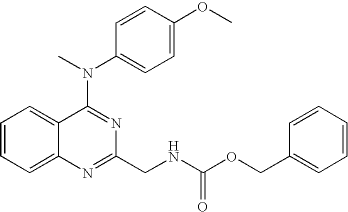

### {4-[(4-Methoxy-phenyl)-methyl-amino]-quinazolin-2-ylmethyl}-carbamic acid benzyl ester

The title compound was produced in a manner similar to that for the compound of Example 46. ¹H NMR (DMSO-d₆) δ7.7-6.92 (m, 14H), 5.10 (s, 2H), 4.37 (d, 2H), 3.79 (s, 3H), 3.48 (s, 3H); LC-MS (ESI⁺; 429 ([M+H]⁺).

### Example 48

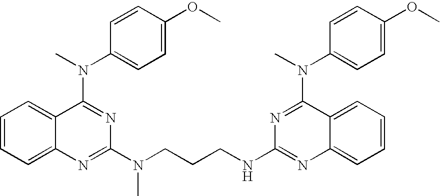

### 3-{4-[(4-Methoxy-phenyl)-methyl-amino]-quinazolin-2-ylamino}-1-({4-[(4-methoxy-phenyl)-methyl-amino]-quinazolin-2-yl}-methyl-amino)-propane

A mixture of (2-chloro-quinazolin-4-yl)-(4-methoxy-phenyl)-methyl-amine hydrochloride (203 mg, 0.604 mmol), ethyl-diisopropyl-amine (0.25 mL, 1.4 mmol) and N¹-methyl-propane-1,3-diamine (95%; 100 μL) in n-butanol (4 mL) was heated at 115° C. After 32 h, the reaction was concentrated then dissolved in CHCl₃, adsorbed onto diatomaceous earth and partially purified on an amine column (Isco 68-2203-101) eluting with a gradient of 0->100% i-PrOH in CHCl₃. Pure title compound was obtained as a tan oil (50 mg; 13%) after pTLC (SiO₂/100:10:1 CHCl₃:MeOH:concd NH₄OH). 1H NMR (DMSO-d6) δ 8.32 (s, 1H), 7.37-6.46 (m, 16H), 3.85 (t(br), 2H), 3.76 (s, 6H), 3.50-3.30 (m, 8H), 3.22 (s, 3H), 1.95 (m, 2H); LC-MS (ESI⁺; 615 ([M+H]⁺).

### Example 49

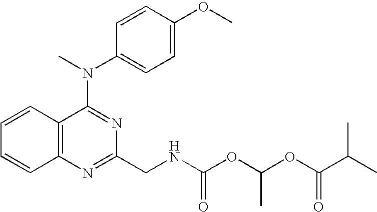

### Isobutyric acid 1-{4-[(4-methoxy-phenyl)-methyl-amino]-quinazolin-2-ylmethylcarbamoyloxy}-ethyl ester

{4-[(4-Methoxy-phenyl)-methyl-amino]-quinazolin-2-ylmethyl}-carbamic acid 1-chloro-ethyl ester: A solution of (2-aminomethyl-quinazolin-4-yl)-(4-methoxy-phenyl)-methyl-amine (250 mg, 0.85 mmol) and N,N,N′,N′-tetramethyl-naphthalene-1,8-diamine (218 mg, 1.02 mmol) in CH₂Cl₂, cooled to 0° C., was treated with 0.10 mL of 1-chloroethyl chloroformate (0.10 mL, 0.93 mmol) and stirred for 1 h. The reaction was warmed to rt and stirred overnight. Another 0.10 mL of the chloroformate was added and the reaction stirred for 2 h. Another 0.10 mL of chloroformate was added and the reaction stirred for an additional 2 h. The reaction was concentrated onto SiO₂ and purified by gradient MPLC (SiO₂, i-PrOH/CH₂Cl₂, 0-100%) to yield 177 mg of the title compound. ¹H NMR (CDCl₃) δ 7.75 (d, 1H), 7.56 (ddd, 1H), 7.10-7.15 (m, 2H), 6.97-7.04 (m, 2H), 6.90-6.95 (m, 2H), 6.69 (q, 1H), 6.45 (s(br), 1H), 4.62 (dd, 2H), 3.85 (s, 3H), 3.59 (s, 3H), 1.86 (d, 3H); HRMS (ES) calcd for C₂₀H₂₂ClN₄O₃ 401.1380, found 401.1343.

Isobutyric acid 1-{4-[(4-methoxy-phenyl)-methyl-amino]-quinazolin-2-ylmethylcarbamoyloxy}-ethyl ester: A suspension of {4-[(4-methoxy-phenyl)-methyl-amino]-quinazolin-2-ylmethyl}-carbamic acid 1-chloro-ethyl ester (101 mg, 0.25 mmol), iso-butyric acid (0.03 mL, 0.32 mmol) and Cs₂CO₃ (111 mg, 0.34 mmol) in THF (2 mL) was stirred at rt for 60 h. The reaction was concentrated onto SiO₂ and purified by gradient MPLC (SiO₂, EtOAc/hexanes, 0-100%) to provide the title compound. ¹H NMR (CDCl₃) δ 7.75 (d, 1H), 7.55 (ddd, 1H), 7.09-7.18 (m, 2H), 6.97-7.05 (m, 2H), 6.88-6.97 (m, 2H), 6.27-6.37 (m, 1H), 4.53-4.66 (m, 2H), 3.85 (s, 3H), 3.58 (s, 3H), 2.56 (sept, 1H), 1.53 (d, 3H), 1.17 (d, 6H); HRMS (ES) calcd for C₂₄H₂₉N₄O₅ (M+H) 453.2133, found 453.2104.

### Example 50

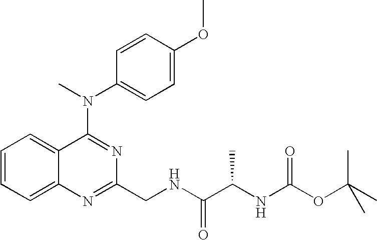

### [(S)-1-({4-[(4-Methoxy-phenyl)-methyl-amino]-quinazolin-2-ylmethyl}-carbamoyl)-ethyl]-carbamic acid tent-butyl ester

To (2-aminomethyl-quinazolin-4-yl)-(4-methoxy-phenyl)-methyl-amine (210 mg, 0.74 mmol), Boc-L-Ala-OH (140 mg, 0.74 mmol), EDCI (170 mg, 0.89 mmol) and HOBt-hydrate (136 mg, 0.89 mmol) in DMF (3 mL) was added ethyl-diisopropyl-amine (383 μL, 2.2 mmol). The reaction was stirred at room temperature overnight, diluted with EtOAc and quenched with 10% aqueous HCl followed by saturated aqueous NaHCO₃. The aqueous phase was extracted with EtOAc (4×15 mL). The combined organic phases were washed with satd NaCl, dried over MgSO₄ and concentrated. Purification by silica gel gradient MPLC (0-20% MeOH/CH₂Cl₂ with 0.1% NH₄OH gave 285 mg of the title compound (83%) as a yellow solid. ¹H NMR (MeOH-d₄) δ 7.69 (d, 1H), 7.61-7.57 (m, 1H), 7.17 (d, 2H), 7.03-6.98 (m, 4H), 4.61-4.49 (m, 2H), 4.23 (q, 1H), 3.83 (s, 3H), 3.62 (s, 3H), 1.44 (s, 9H), 1.41 (d, 3H); LC-MS (ESI⁺; 466 ([M+H]⁺).

### Example 51

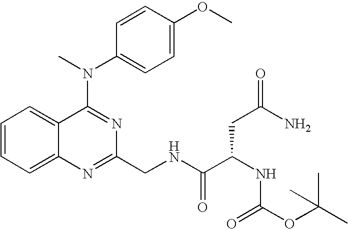

### [(S)-2-Carbamoyl-1-({4-[(4-methoxy-phenyl)-methyl-amino]-quinazolin-2-ylmethyl}-carbamoyl)-ethyl]-carbamic acid tent-butyl ester

The title compound was prepared from (2-aminomethyl-quinazolin-4-yl)-(4-methoxy-phenyl)-methyl-amine (200 mg, 0.67 mmol), Boc-L-Asn-OH (155 mg, 0.67 mmol), EDCI (157 mg, 0.82 mmol), HOBt-hydrate (126 mg, 0.82 mmol) and ethyl-diisopropyl-amine (348 μL, 2.0 mmol) in DMF (3 mL) by a procedure similar to Example 50 and provided 270 mg (79%) of the title compound as a yellow solid. ¹H NMR (MeOH-d₄) δ 7.71 (d, 1H), 7.62-7.58 (m, 1H), 7.18 (d, 2H), 7.04-6.99 (m, 4H), 4.61 (t, 1H), 4.56 (d, 2H), 3.83 (s, 3H), 3.64 (s, 3H), 2.85-2.65 (m, 2H), 1.44 (s, 9H); LC-MS (ESI⁺; 509 ([M+H]⁺).

### Example 52

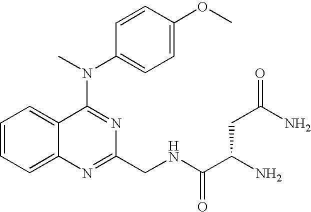

### (S)-2-Amino-N¹-{4-[(4-methoxy-phenyl)-methyl-amino]-quinazolin-2-ylmethyl}-succinamide

To [(S)-2-carbamoyl-1-({4-[(4-methoxy-phenyl)-methyl-amino]-quinazolin-2-ylmethyl}-carbamoyl)-ethyl]-carbamic acid tert-butyl ester (115 mg, 0.226 mmol) in methanol (1 mL) was added concentrated concd HCl (1 mL). The solvent was removed under vacuum and purification by gradient MPLC (SiO₂, 0-100% gradient of (80:20:0.1 CH₂Cl₂:MeOH:concd NH₄OH) in CH₂Cl₂) gave product as a yellow solid (17 mg). ¹H NMR (MeOH-d₄) δ 7.98 (d, 1H), 7.84-7.78 (m, 1H), 7.36 (d, 2H), 7.24 (t, 1H), 7.12 (d, 2H), 6.93 (d, 1H), 4.82 (s, 2H), 4.61 (d, 1H), 4.4 (t, 1H), 4.82 (s, 2H), 3.88 (s, 3H), 3.82 (s, 3H), 3.10 (t, 1H); LC-MS (ESI⁺; 409 ([M+H]⁺).

### Example 53

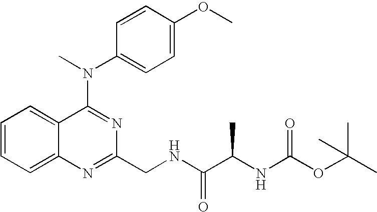

### [(R)-1-({4-[(4-Methoxy-phenyl)-methyl-amino]-quinazolin-2-ylmethyl}-carbamoyl)-ethyl]-carbamic acid tert-butyl ester

The title compound was prepared from (2-aminomethyl-quinazolin-4-yl)-(4-methoxy-phenyl)-methyl-amine (100 mg, 0.37 mmol), Boc-D-Ala-OH (70 mg, 0.37 mmol), EDCI (86 mg, 0.45 mmol), HOBt-hydrate (68 mg, 0.82 mmol) and ethyl-diisopropyl-amine (191 IA, 1.1 mmol) in DMF (1.2 mL) by a procedure similar to the preparation of Example 50 and was isolated as a yellow solid (114 mg; 67%). ¹H NMR (MeOH-d₄) δ 7.7 (d, 1H), 7.62-7.57 (m, 1H), 7.18 (d, 2H), 7.06-6.98 (m, 4H), 4.55 (m, 2H), 4.2 (q, 1H), 3.82 (s, 3H), 3.62 (s, 3H), 1.49 (s, 9H), 1.48 (d, 3H); LC-MS (ESI⁺; 466 ([M+H]⁺).

### Example 54

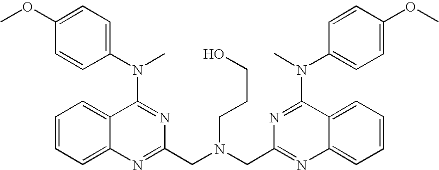

### 3-(Bis-{4-[(4-methoxy-phenyl)-methyl-amino]-quinazolin-2-ylmethyl}-amino)-propan-1-ol

A mixture of (2-chloromethyl-quinazolin-4-yl)-(4-methoxy-phenyl)-methyl-amine hydrochloride (100 mg, 0.286 mmol), Cs₂CO₃ (201 mg, 0.617 mmol) and 3-amino-propan-1-ol (44 μL, 0.58 mmol) in DMF (1 mL) was heated at 80° C. After 18 h, EtOAc (12 mL) was added and this was washed with water (2×2 mL) and satd NaCl (2×2 mL). The soln was dried (MgSO₄), filtered through a plug of silica with a wash of 100:10:1 chloroform:methanol:concd NH₄OH then purified by preparative TLC (SiO₂/200:10:1 chloroform:2-propanol:concd NH₄OH) to yield the title compound as a yellow hard foam. ¹H NMR (CDCl₃) δ 7.81 (m, 2H), 7.52 (m, 2H), 7.07 (m, 4H), 6.98-6.42 (m, 4H), 6.88 (m, 4H), 4.22 (s, 4H), 3.99 (m, 2H), 3.83 (s, 6H), 3.59 (s, 6H), 3.08 (m, 2H), 1.91 (m, 2H); LC-MS (ESI⁺; 630 ([M+H]⁺).

### Example 55

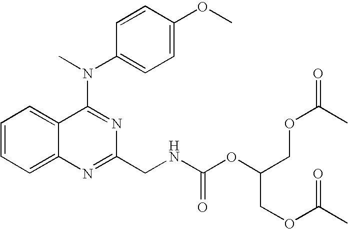

### Acetic acid 3-acetoxy-2-{4-[(4-methoxy-phenyl)-methyl-amino]-quinazolin-2-ylmethylcarbamoyloxy}-propyl ester

A solution of diacetin (0.50 mL, 3.35 mmol), NEt₃ (0.56 mL, 4.0 mmol) in EtOAc (18 mL) was treated with 4-nitrophenyl chloroformate (672 mg, 3.2 mmol) and stirred at rt for 1 h. The reaction was then diluted with EtOAc, washed with satd NaCl, dried (MgSO₄), filtered and concentrated. Purification by MPLC (SiO₂, EtOAc/hexanes, 0-100%) provided the mixed anhydride (as a mixture of diacetin isomers). The mixed anhydride (110 mg, 0.32 mmol) was added to a solution of (2-aminomethyl-quinazolin-4-yl)-(4-methoxy-phenyl)-methyl-amine (64.2 mg, 0.22 mmol) in THF (1 mL). The reaction was stirred for 3 h at rt, concentrated onto SiO₂ and purified by MPLC (SiO₂, EtOAc/hexanes, 0-100%). Further purification by RPLC gave the title compound as a mixture with the isomer acetic acid 1-acetoxymethyl-2-{4-[(4-methoxy-phenyl)-methyl-amino]-quinazolin-2-ylmethylcarbamoyloxy}-ethyl ester. ¹H NMR (CDCl₃) δ 7.76 (d, 1H), 7.52-7.60 (m, 1H), 7.08-7.18 (m, 2H), 6.98-7.04 (m, 2H), 6.88-6.98 (m, 2H), 6.10-6.38 (m, 1H), 5.22-5.38 (m, 1H), 4.45-4.65 (m, 2H), 4.36-4.42 (m, 4H), 3.85 (s, 3H), 3.58 (s, 3H), 2.05-2.15 (m, 6H); LC-MS (ESI⁺; 497 ([M+H]⁺).

### Example 56

### Identification of Cytotoxic Agents

A P388 murine leukemia cell line was obtained from NCI, Frederick, Md. P388 cells were cultured in RPMI-1640 supplemented with 10% fetal bovine serum, 2 mM Glutamax, 1 mM sodium pyruvate, 0.1 mM non-essential amino acids and 10 mM HEPES. Cells were grown at 37° C. in a humidified 5% CO₂ atmosphere. Exponentially growing P388 cells were plated at 5,000 cells/well in a 96-well flat-bottomed microtiter plate (Corning, Costar #3595). Twenty-four hours later, test compound was added to cells at final concentrations of 100 nM, 33.3 nM, 11.1 nM, 3.7 nM, 1.23 nM, 0.4 nM and 0.13 nM. Cellular viability was determined 72 hours later by measuring intracellular ATP with ATP-Lite assay system. The effect of compounds on cell viability was calculated by comparing the ATP levels of cells exposed to test compound with those of cells exposed to DMSO. A semi-log plot of relative ATP levels versus compound concentration was used to calculate the compound concentration required to inhibit growth by 50% (IC₅₀). Data was analyzed by Prism software (GraphPad; San Diego, Calif.) by fitting it to a sigmoidal dose response curve.

The P388 IC₅₀ data of representative compounds are summarized in Table I:

TABLE I
P388 IC₅₀ Data

| Example Cmpd No. | P388 IC₅₀ (nM) |
|---|---|
| 1 | 490 |
| 2 | 65 |
| 3 | 1400 |
| 4 | 550 |
| 5 | 3600 |
| 6 | 3000 |
| 7 | 1400 |
| 8 | 25 |
| 9 | 2600 |
| 10 | 370, 2100, 1400 |
| 11 | 22, 7 |
| 12 | 3100 |
| 13 | 15 |
| 14 | 300, 340 |
| 15 | 11, 15 |
| 16 | 4000, 2400 |
| 17 | 4500, 3000 |
| 18 | 1900, 730 |
| 19 | 1700, 530 |
| 20 | 1700, 1100 |
| 21 | 120, 64 |
| 22 | 9300 |
| 23 | 4600 |
| 24 | 1600 |
| 25 | 1800 |
| 26 | 7200 |
| 27 | 220 |
| 28 | 150 |
| 29 | 32 |
| 30 | 85 |
| 31 | 1300 |
| 32 | 240 |
| 33 | 17 |
| 34 | 36 |
| 35 | 220 |
| 36 | 780 |
| 37 | 4700 |
| 38 | 19 |
| 39 | 3800 |
| 40 | 500 |
| 41 | 1700 |
| 42 | 680 |
| 43 | 7.2 |
| 44 | 37 |
| 45 | 1700 |
| 46 | 6.6, 22 |
| 47 | 23 |
| 48 | 70 |
| 49 | 5.5 |
| 50 | 180 |
| 51 | 620 |
| 52 | 73, 35 |
| 53 | 457 |
| 54 | 64 |
| 55 | 330 |

Accordingly, compounds of the invention were identified as cytotoxic agents and are thus useful in treating the various diseases and disorders discussed above

### Example 57

### Multidrug Resistant Cell Assays

Cytotoxicity of compounds in multidrug resistant cells can be determined by administering compounds to cell lines that overexpress the multidrug resistance pump MDR-1 and determining the viability of the cell lines. P388/ADR cell lines are known to overexpress the multidrug resistance pump MDR-1 (also known as P-glycoprotein-1; Pgp-1).

P388/ADR cell lines are obtained from American Type Culture Collection (Manassas, Va.) and maintained in RPMI-1640 media supplemented with 10% FCS, 10 units/mL penicillin and streptomycin, 2 mM Glutamax and 1 mM sodium pyruvate (Invitrogen Corporation, Carlsbad, Calif.). For compound testing, cells are plated in 96 well dishes at a concentration of 1.5×10⁴ cells/well. Cells are allowed to adhere to the plate overnight and then incubated with compounds at final concentrations ranging from 0.13 nM to 10 μM for 72 hours. Cell viability is then assessed using the ATP-lite reagent (Perkin Elmer, Foster City, Calif.). Plates are read on a Wallac Topcount luminescence reader (Perkin Elmer, Foster City, Calif.) and the results graphed in Prism software (Graphpad Software, Inc., San Diego, Calif.). Non-linear regression with variable slope analysis is performed to obtain IC₅₀ concentration values.

The P388/MDR IC₅₀ data of representative compounds are summarized in Table II:

TABLE II
P388/MDR IC₅₀ Data

| Example Cmpd No. | P388/MDR IC₅₀ (nM) |
|---|---|
| 1 | 580 |
| 2 | 74 |
| 3 | 2000 |
| 4 | 530 |
| 6 | 3500 |
| 7 | 520 |
| 8 | 30 |
| 9 | 3200 |
| 10 | 380 |
| 11 | 21, 6.3 |
| 12 | 7800 |
| 13 | 18 |
| 14 | 340, 350 |
| 15 | 33, 22 |
| 16 | 3400, 2000 |
| 17 | 8500 |
| 18 | 2500, 1400 |
| 19 | 1500, 580 |
| 20 | 9400 |
| 21 | 190, 150 |
| 23 | 3300 |
| 24 | 1700 |
| 25 | 1800 |
| 26 | 7400 |
| 27 | 220 |
| 28 | 170 |
| 29 | 40 |
| 30 | 160 |
| 31 | 1100 |
| 32 | 300 |
| 33 | 18 |
| 34 | 43 |
| 35 | 240 |
| 36 | 700 |
| 37 | 6600 |
| 38 | 53 |
| 40 | 600 |
| 41 | 5300 |
| 42 | 880 |
| 43 | 5.7 |
| 44 | 57 |
| 45 | 1800 |
| 46 | 8, 18 |
| 47 | 27 |
| 48 | 83 |
| 49 | 13 |
| 50 | 190 |
| 51 | 970 |
| 52 | 222, 60 |
| 53 | 680 |
| 54 | 76 |
| 55 | 450 |

Accordingly, compounds of the invention were identified as cytotoxic agents in multidrug resistant cells and are thus useful in treating the various diseases and disorders discussed above in drug resistant cancer patients.

### Example 58

### Injection Formulation

| Excipients | Amount | |
|---|---|---|
| Active Compound | 5 | mg |
| PEG-400 | 5 | grams |
| TPGS | 10 | grams |
| Benzyl alcohol | 0.5 | gram |
| Ethanol | 2 | grams |
| D5W | Add to make 50 | mL |

An injection formulation of a compound selected from Formula I (the “Active Compound”) can be prepared according to the following method. 5 mg of the Active Compound is dissolved into a mixture of the d-α-tocopheryl polyethylene glycol 1000 succinate (TPGS), PEG-400, ethanol, and benzyl alcohol. D5W is added to make a total volume of 50 mL and the solution is mixed. The resulting solution is filtered through a 0.2 μm disposable filter unit and is stored at 25° C. Solutions of varying strengths and volumes are prepared by altering the ratio of Active Compound in the mixture or changing the total amount of the solution.

### Example 59

### Tablet Formulation

| | |
|---|---|
| Active Compound | 100.0 mg |
| Lactose | 100.0 mg |
| Corn Starch | 50.0 mg |
| Hydrogenated Vegetable Oil | 10.0 mg |
| Polyvinylpyrrolidone | 10.0 mg |
| | 270.0 mg |

A formulation of tablets of a compound selected from Formula I (the “Active Compound”) can be prepared according to the following method. 100 mg of Active Compound) is mixed with 100 mg lactose. A suitable amount of water for drying is added and the mixture is dried. The mixture is then blended with 50 mg of corn starch, 10 mg hydrogenated vegetable oil, and 10 mg polyvinylpyrrolidinone. The resulting granules are compressed into tablets. Tablets of varying strengths are prepared by altering the ratio of Active Compound in the mixture or changing the total weight of the tablet.

### Example 60

### Capsule Formulation

| | |
|---|---|
| Active Compound | 100.0 mg |
| Microcrystalline Cellulose | 200.0 mg |
| Corn Starch | 100.0 mg |
| Magnesium Stearate | 400.0 mg |
| | 800.0 mg |

A formulation of capsules containing 100.0 mg of a compound selected from Formula I (the “Active Compound”) can be prepared according to the following method. 100 mg of Active Compound is mixed with 200 mg of microcrystalline cellulose and 100 mg of corn starch. 400 mg of magnesium stearate is then blended into the mixture and the resulting blend is encapsulated into a gelatin capsule. Doses of varying strengths can be prepared by altering the ratio of the Active Compound to pharmaceutically acceptable carriers or changing the size of the capsule.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A compound according to Formula I: 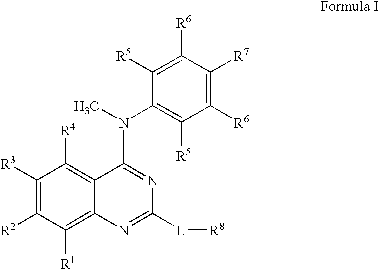
or a pharmaceutically acceptable salt thereof, wherein,
R¹, R², R³, R⁴, R⁵, and R⁶ are each independently chosen from the group consisting of H, C₁₋₃alkyl, C₁₋₃alkoxy, and halo;
R⁷ is C₁₋₃alkoxy; and
L is chosen from —OCH₂CH₂—, —NHCH₂CH₂—, —CH₂NHC(═O)—, —CH₂NHS(═O)₂—, or —CH₂OS(═O)₂—; each optionally substituted with one or more H or C₁₋₃alkyl; with R⁸ chosen from H, OH, amino, 2,6-diamino-hexanoic acid, 2-amino-3-(1H-indol-3-yl)-propionic acid, 2,6-diamino-hexanoic acid, 2-amino-4-methylsulfanyl-butyric acid, 2-amino-succinamic acid, 2-benzyloxycarbonylamino-3-(1H-indol-3-yl)-propionic acid, 2-(2-amino-ethoxy)-ethanol, 2-(2-ethoxy-ethoxy)-ethanol, 2-(2-methoxy-ethoxy)-ethanol, 2-amino-acetamide, 2-methoxy-ethanol, 2-piperazin-1-yl-ethanol, 8-methyl-1,3-dioxo-2,3,5,8-tetrahydro-1H-[1,2,4]triazolo[1,2-a]pyridazine-5-carboxylic acid ethyl ester, acetamide, amino-acetic acid, carbamic acid methyl ester, carbamic acid tert-butyl ester, carbamoylmethyl-carbamic acid tert-butyl ester, carbonic acid mono-[2-(2-methoxy-ethoxy)-ethyl]ester, ethyl-carbamic acid tert-butyl ester, isobutyric acid 1-hydroxy-ethyl ester, methoxy-benzene, methyl-urea, N¹-(2-amino-ethyl)-ethane-1,2-diamine, phenol, phenyl-methanol, phosphoric acid di-tent-butyl ester, phosphoric acid mono-tent-butyl ester, (2-carbamoyl-ethyl)-carbamic acid tert-butyl ester, 3-amino-propionamide, acetic acid 3-acetoxy-2-hydroxy-propyl ester, or urea; each optionally substituted with one or more halo, N₃, OH, thiol, nitro, CN, C₁₋₆alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle; or
L is a direct bond; with R⁸ chosen from 3-{4-[(4-methoxy-phenyl)-methyl-amino]-quinazolin-2-ylmethyl}-methyl-amino)-propan-1-ol, N⁴-(4-methoxy-phenyl)-N⁴-methyl-N²-(3-methylamino-propyl)-quinazoline-2,4-diamine, 2-amino-1-phenyl-propan-1-ol, 2-amino-2-phenyl-ethanol, 2-amino-3-(4-hydroxy-phenyl)-propionic acid methyl ester, 2-amino-3-phenyl-propan-1-ol, 2-amino-propionic acid, 2-amino-propionic acid tent-butyl ester, 2-amino-3-methyl-butyric acid ethyl ester, N-(2-Acetylamino-ethyl)-acetamide, 1-amino-3-{4-[(4-methoxy-phenyl)-methyl-amino]-quinazolin-2-ylamino}-propan-2-ol, 1,3-diamino-propan-2-ol, or piperazine; optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle.

2. The compound of claim 1, wherein R¹, R², R⁴, and R⁵ are each independently H or C₁₋₃alkyl.

3. The compound of claim 1, wherein R³ is chosen from —OCH₃, —CH₃, and F.

4. The compound of claim 1, wherein R⁶ is independently chosen from H and C₁₋₃alkoxy.

5. The compound of claim 1, wherein R⁷ is —OCH₃.

6. The compound of claim 1, wherein L is chosen from —OCH₂CH₂—, —NHCH₂CH₂—, —CH₂NHC(═O)—, or —CH₂NHS(═O)₂—; with R⁸ chosen from H, (R)-2,6-diamino-hexanoic acid, (R)-2-amino-3-(1H-indol-3-yl)-propionic acid, (S)-2,6-diamino-hexanoic acid, (S)-2-amino-4-methylsulfanyl-butyric acid, (S)-2-amino-succinamic acid, (S)-2-benzyloxycarbonylamino-3-(1H-indol-3-yl)-propionic acid, 2-(2-amino-ethoxy)-ethanol, 2-(2-ethoxy-ethoxy)-ethanol, 2-(2-methoxy-ethoxy)-ethanol, 2-amino-acetamide, 2-methoxy-ethanol, 2-piperazin-1-yl-ethanol, 8-methyl-1,3-dioxo-2,3,5,8-tetrahydro-1H-[1,2,4]triazolo[1,2-a]pyridazine-5-carboxylic acid ethyl ester, acetamide, amino-acetic acid, carbamic acid methyl ester, carbamic acid tert-butyl ester, carbamoylmethyl-carbamic acid tert-butyl ester, carbonic acid mono-[2-(2-methoxy-ethoxy)-ethyl]ester, ethyl-carbamic acid tert-butyl ester, isobutyric acid 1-hydroxy-ethyl ester, methoxy-benzene, methyl-urea, phenol, phenyl-methanol, phosphoric acid di-tent-butyl ester, phosphoric acid mono-tent-butyl ester, (2-carbamoyl-ethyl)-carbamic acid tert-butyl ester, 3-amino-propionamide, acetic acid 3-acetoxy-2-hydroxy-propyl ester, or urea; optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle.

7. The compound of claim 1, wherein L is a direct bond and R⁸ chosen from N⁴-(4-methoxy-phenyl)-N⁴-methyl-N²-(3-methylamino-propyl)-quinazoline-2,4-diamine, (1R,2S)-2-amino-1-phenyl-propan-1-ol, (S)-2-amino-2-phenyl-ethanol, (S)-2-amino-3-(4-hydroxy-phenyl)-propionic acid methyl ester, (S)-2-amino-3-phenyl-propan-1-ol, (S)-2-amino-propionic acid, (S)-2-amino-propionic acid tert-butyl ester, 2-amino-3-methyl-butyric acid ethyl ester, N-(2-acetylamino-ethyl)-acetamide, 1-amino-3-{4-[(4-methoxy-phenyl)-methyl-amino]-quinazolin-2-ylamino}-propan-2-ol, 1,3-diamino-propan-2-ol, or piperazine; optionally substituted with one or more halo, N₃, OH, thiol, nitro, CN, C₁₋₆alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle.

8. The compound of claim 1, wherein L is —NHCH₂CH₂—, and R⁸ is chosen from 2-(2-amino-ethoxy)-ethanol, N¹-(2-amino-ethyl)-ethane-1,2-diamine, 8-methyl-1,3-dioxo-2,3,5,8-tetrahydro-1-[1,2,4]triazolo[1,2-a]pyridazine-5-carboxylic acid ethyl ester, carbonic acid mono-[2-(2-methoxy-ethoxy)-ethyl]ester, phosphoric acid di-tent-butyl ester, (S)-2,6-diamino-hexanoic acid, phosphoric acid mono-tent-butyl ester, amino-acetic acid, carbamic acid tert-butyl ester, (R)-2,6-diamino-hexanoic acid, carbamic acid benzyl ester, carbamic acid methyl ester, (S)-2-amino-4-methylsulfanyl-butyric acid, (S)-2-benzyloxycarbonylamino-3-(1H-indol-3-yl)-propionic acid, carbamoylmethyl-carbamic acid tert-butyl ester, (S)-2-amino-succinamic acid, (R)-2-amino-3-(1H-indol-3-yl)-propionic acid, methyl-urea, 2-amino-acetamide, acetamide, and urea.

9. The compound of claim 1, wherein L is —OCH₂CH₂—, and R⁸ is chosen from 2-methoxy-ethanol, 2-(2-methoxy-ethoxy)-ethanol, 2-piperazin-1-yl-ethanol, and 2-(2-ethoxy-ethoxy)-ethanol, or wherein L is —CH₂NHC(═O)—, and R⁸ is chosen from ethyl-carbamic acid tert-butyl ester, isobutyric acid 1-hydroxy-ethyl ester, phenyl-methanol, and phenol, or wherein L is —CH₂NHS(═O)₂—, and R⁸ is methoxy-benzene.

10. A pharmaceutical composition comprising a pharmaceutically acceptable carrier and an effective amount of a compound according to claim 1.

11. The pharmaceutical composition of claim 10, further comprising at least one cancer chemotherapeutic agent other than the compound of claim 1, or a pharmaceutically acceptable salt of said at least one cancer chemotherapeutic agent.

12. The pharmaceutical composition of claim 11, wherein said at least one cancer chemotherapeutic agent is selected from alkylating agents, antimitotic agents, topoisomerase I inhibitors, topoisomerase II inhibitors, RNA/DNA antimetabolites, DNA antimetabolites, EGFR inhibitors, proteosome inhibitors, antibodies, and combinations thereof.

13. A compound selected from the group consisting of:
{2-[2-(2-methoxy-ethoxy)-ethoxy]-quinazolin-4-yl}-(4-methoxy-phenyl)-methyl-amine;
(2-{2-[2-(2-methoxy-ethoxy)-ethoxy]-ethoxy}-quinazolin-4-yl)-(4-methoxy-phenyl)-methyl-amine;
N²-{2-[2-(2-amino-ethoxy)-ethoxy]-ethyl}-N⁴-(4-methoxy-phenyl)-N⁴-methyl-quinazoline-2,4-diamine;
N²-{2-[2-(2-amino-ethylamino)-ethylamino]-ethyl}-N⁴-(4-methoxy-phenyl)-N⁴-methyl-quinazoline-2,4-diamine;
2-[4-(2-{4-[(4-methoxy-phenyl)-methyl-amino]-quinazolin-2-yloxy}-ethyl)-piperazin-1-yl]-ethanol;
2-(2-{4-[(4-methoxy-phenyl)-methyl-amino]-quinazolin-2-ylamino}-ethyl)-8-methyl-1,3-dioxo-2,3,5,8-tetrahydro-1H-[1,2,4]triazolo[1,2-a]pyridazine-5-carboxylic acid ethyl ester;
(2-{2-[2-(2-ethoxy-ethoxy)-ethoxy]-ethoxy}-quinazolin-4-yl)-(4-methoxy-phenyl)-methyl-amine;
carbonic acid 2-(2-methoxy-ethoxy)-ethyl ester 2-{4-[(4-methoxy-phenyl)-methyl-amino]-quinazolin-2-ylamino}-ethyl ester;
(4-methoxy-phenyl)-methyl-(2-piperazin-1-yl-quinazolin-4-yl)-amine;
phosphoric acid di-tent-butyl ester 2-{4-[(4-methoxy-phenyl)-methyl-amino]-quinazolin-2-ylamino}-ethyl ester;
(S)-2,6-diamino-hexanoic acid 2-{4-[(4-methoxy-phenyl)-methyl-amino]-quinazolin-2-ylamino}-ethyl ester;
phosphoric acid tent-butyl ester 2-{4[(4-methoxy-phenyl)-methyl-amino]-quinazolin-2-ylamino}-ethyl ester;
amino-acetic acid 2-{4-[(4-methoxy-phenyl)-methyl-amino]-quinazolin-2-ylamino}-ethyl ester;
(2-{4-[(3,4-dimethoxy-phenyl)-methyl-amino]-quinazolin-2-ylamino}-ethyl)-carbamic acid tert-butyl ester;
(R)-2,6-diamino-hexanoic acid 2-{4-[(4-methoxy-phenyl)-methyl-amino]-quinazolin-2-ylamino}-ethyl ester;
(2-{6-methoxy-4-[(4-methoxy-phenyl)-methyl-amino]-quinazolin-2-ylamino}-ethyl)-carbamic acid tert-butyl ester;
(2-{6-methoxy-4-[(4-methoxy-phenyl)-methyl-amino]-quinazolin-2-ylamino}-ethyl)-carbamic acid benzyl ester;
N-(2-{4-[(4-methoxy-phenyl)-methyl-amino]-6-methyl-quinazolin-2-ylamino}-ethyl)-acetamide;
(2-{4-[(4-methoxy-phenyl)-methyl-amino]-quinazolin-2-ylamino}-ethyl)-carbamic acid tert-butyl ester;
N-(2-{4-[(3,4-dimethoxy-phenyl)-methyl-amino]-quinazolin-2-ylamino}-ethyl)-acetamide;
(2-{4-[(3,4-dimethoxy-phenyl)-methyl-amino]-quinazolin-2-ylamino}-ethyl)-carbamic acid methyl ester;
(2-{6-methoxy-4-[(4-methoxy-phenyl)-methyl-amino]-quinazolin-2-ylamino}-ethyl)-urea;
(S)-2-{4-[(4-methoxy-phenyl)-methyl-amino]-quinazolin-2-ylamino}-2-phenyl-ethanol;
(1R,2S)-2-{4-[(4-methoxy-phenyl)-methyl-amino]-quinazolin-2-ylamino}-1-phenyl-propan-1-ol;
(S)-2-{4-[(4-methoxy-phenyl)-methyl-amino]-quinazolin-2-ylamino}-3-phenyl-propan-1-ol;
(2-{6-methoxy-4-[(4-methoxy-phenyl)-methyl-amino]-quinazolin-2-ylamino}-ethyl)-carbamic acid methyl ester;
(S)-2-amino-4-methylsulfanyl-butyric acid 2-{4-[(4-methoxy-phenyl)-methyl-amino]-quinazolin-2-ylamino}-ethyl ester;
(S)-2-{4-[(4-methoxy-phenyl)-methyl-amino]-quinazolin-2-ylamino}-propionic acid tert-butyl ester;
(S)-2-benzyloxycarbonylamino-3-(1H-indol-3-yl)-propionic acid 2-{4-[(4-methoxy-phenyl)-methyl-amino]-quinazolin-2-ylamino}-ethyl ester;
(2-{4-[(4-methoxy-phenyl)-methyl-amino]-6-methyl-quinazolin-2-ylamino}-ethyl)-carbamic acid methyl ester;
[(2-{4-[(4-methoxy-phenyl)-methyl-amino]-quinazolin-2-ylamino}-ethylcarbamoyl)-methyl]-carbamic acid tent-butyl ester;
2-{4-[(4-methoxy-phenyl)-methyl-amino]-quinazolin-2-ylamino}-3-methyl-butyric acid ethyl ester;
(S)-2-amino-succinamic acid 2-{4-[(4-methoxy-phenyl)-methyl-amino]-quinazolin-2-ylamino}-ethyl ester;
(S)-3-(4-hydroxy-phenyl)-2-{4-[(4-methoxy-phenyl)-methyl-amino]-quinazolin-2-ylamino}-propionic acid methyl ester;
(R)-2-amino-3-(1H-indol-3-yl)-propionic acid 2-{4-[(4-methoxy-phenyl)-methyl-amino]-quinazolin-2-ylamino}-ethyl ester;
(S)-2-{4-[(4-methoxy-phenyl)-methyl-amino]-quinazolin-2-ylamino}-propionic acid;
1-(2-{6-methoxy-4-[(4-methoxy-phenyl)-methyl-amino]-quinazolin-2-ylamino}-ethyl)-3-methyl-urea;
(2-{4-[(4-methoxy-phenyl)-methyl-amino]-quinazolin-2-ylamino}-ethyl)-carbamic acid methyl ester;
N-[2-(acetyl-{4-[(4-methoxy-phenyl)-methyl-amino]-quinazolin-2-yl}-amino)-ethyl]-acetamide;
(2-{4-[(4-methoxy-phenyl)-methyl-amino]-quinazolin-2-ylamino}-ethyl)-urea;
2-Amino-N-(2-{4-[(4-methoxy-phenyl)-methyl-amino]-quinazolin-2-ylamino}-ethyl)-acetamide;
1,3-bis-{4-[(4-methoxy-phenyl)-methyl-amino]-quinazolin-2-ylamino}-propan-2-ol;
{6-fluoro-4-[(4-methoxy-phenyl)-methyl-amino]-quinazolin-2-ylmethyl}-carbamic acid phenyl ester;
1-amino-3-{4-[(4-methoxy-phenyl)-methyl-amino]-quinazolin-2-ylamino}-propan-2-ol;
4-methoxy-N-{4-[(4-methoxy-phenyl)-methyl-amino]-quinazolin-2-ylmethyl}-benzenesulfonamide;
{4-[(4-methoxy-phenyl)-methyl-amino]-quinazolin-2-ylmethyl}-carbamic acid phenyl ester;
{4-[(4-methoxy-phenyl)-methyl-amino]-quinazolin-2-ylmethyl}-carbamic acid benzyl ester;
3-{4-[(4-methoxy-phenyl)-methyl-amino]-quinazolin-2-ylamino}-1-({4-[(4-methoxy-phenyl)-methyl-amino]-quinazolin-2-yl}-methyl-amino)-propane;
isobutyric acid 1-{4-[(4-methoxy-phenyl)-methyl-amino]-quinazolin-2-ylmethylcarbamoyloxy}-ethyl ester;
[1-({4-[(4-methoxy-phenyl)-methyl-amino]-quinazolin-2-ylmethyl}-carbamoyl)-ethyl]-carbamic acid tent-butyl ester;
[(S)-2-carbamoyl-1-({4-[(4-methoxy-phenyl)-methyl-amino]-quinazolin-2-ylmethyl}-carbamoyl)-ethyl]-carbamic acid tert-butyl ester;
(S)-2-amino-N*1*-{4-[(4-methoxy-phenyl)-methyl-amino]-quinazolin-2-ylmethyl}-succinamide;
[(R)-1-({4-[(4-methoxy-phenyl)-methyl-amino]-quinazolin-2-ylmethyl}-carbamoyl)-ethyl]-carbamic acid tert-butyl ester;
3-(bis-{4-([4-methoxy-phenyl)-methyl-amino]-quinazolin-2-ylmethyl}-amino)-propan-1-ol; and
acetic acid 3-acetoxy-2-{4-[(4-methoxy-phenyl)-methyl-amino]-quinazolin-2-ylmethylcarbamoyloxy}-propyl ester;
or a pharmaceutically acceptable salt thereof.

14. A pharmaceutical composition comprising a pharmaceutically acceptable carrier and an effective amount of a compound according to claim 13.

15. A method of treating cancer comprising treating cells in vitro or a warm-blooded animal with an effective amount of a compound according to Formula I: 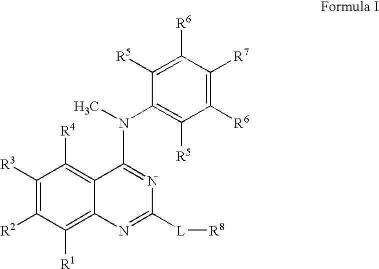
or a pharmaceutically acceptable salt thereof, wherein,
R¹, R², R³, R⁴, R⁵, and R⁶ are each independently chosen from the group consisting of H, C₁₋₃alkyl, C₁₋₃alkoxy, and halo;
R⁷ is C₁₋₃alkoxy; and
L is chosen from —OCH₂CH₂—, —NHCH₂CH₂—, —CH₂NHC(═O)—, —CH₂NHS(═O)₂—, or —CH₂OS(═O)₂—; each optionally substituted with one or more H or C₁₋₃alkyl; with R⁸ chosen from H, OH, amino, 2,6-diamino-hexanoic acid, 2-amino-3-(1H-indol-3-yl)-propionic acid, 2,6-diamino-hexanoic acid, 2-amino-4-methylsulfanyl-butyric acid, 2-amino-succinamic acid, 2-benzyloxycarbonylamino-3-(1H-indol-3-yl)-propionic acid, 2-(2-amino-ethoxy)-ethanol, 2-(2-ethoxy-ethoxy)-ethanol, 2-(2-methoxy-ethoxy)-ethanol, 2-amino-acetamide, 2-methoxy-ethanol, 2-piperazin-1-yl-ethanol, 8-methyl-1,3-dioxo-2,3,5,8-tetrahydro-1H-[1,2,4]triazolo[1,2-a]pyridazine-5-carboxylic acid ethyl ester, acetamide, amino-acetic acid, carbamic acid methyl ester, carbamic acid tert-butyl ester, carbamoylmethyl-carbamic acid tert-butyl ester, carbonic acid mono-[2-(2-methoxy-ethoxy)-ethyl]ester, ethyl-carbamic acid tert-butyl ester, isobutyric acid 1-hydroxy-ethyl ester, methoxy-benzene, methyl-urea, N¹-(2-amino-ethyl)-ethane-1,2-diamine, phenol, phenyl-methanol, phosphoric acid di-tent-butyl ester, phosphoric acid mono-tent-butyl ester, (2-carbamoyl-ethyl)-carbamic acid tert-butyl ester, 3-amino-propionamide, acetic acid 3-acetoxy-2-hydroxy-propyl ester, or urea; each optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle; or
L is a direct bond; with R⁸ chosen from 3-({4-[(4-methoxy-phenyl)-methyl-amino]-quinazolin-2-ylmethyl}-methyl-amino)-propan-1-ol, N⁴-(4-methoxy-phenyl)-N⁴-methyl-N²-(3-methylamino-propyl)-quinazoline-2,4-diamine, 2-amino-1-phenyl-propan-1-ol, 2-amino-2-phenyl-ethanol, 2-amino-3-(4-hydroxy-phenyl)-propionic acid methyl ester, 2-amino-3-phenyl-propan-1-ol, 2-amino-propionic acid, 2-amino-propionic acid tert-butyl ester, 2-amino-3-methyl-butyric acid ethyl ester, N-(2-acetylamino-ethyl)-acetamide, 1-amino-3-{4-[(4-methoxy-phenyl)-methyl-amino]-quinazolin-2-ylamino}-propan-2-ol, 1,3-diamino-propan-2-ol, or piperazine; optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle.

16. The method of claim 15, wherein R', R², R⁴, and R⁵ are each independently H or C₁₋₃ alkyl.

17. The method of claim 15, wherein L is chosen from —OCH₂CH₂—, —NHCH₂CH₂—, —CH₂NHC(═O)—, or —CH₂NHS(═O)₂—; with R⁸ chosen from H, (R)-2,6-diamino-hexanoic acid, (R)-2-amino-3-(1H-indol-3-yl)-propionic acid, (S)-2,6-diamino-hexanoic acid, (S)-2-amino-4-methylsulfanyl-butyric acid, (S)-2-amino-succinamic acid, (S)-2-benzyloxycarbonylamino-3-(1H-indol-3-yl)-propionic acid, 2-(2-amino-ethoxy)-ethanol, 2-(2-ethoxy-ethoxy)-ethanol, 2-(2-methoxy-ethoxy)-ethanol, 2-amino-acetamide, 2-methoxy-ethanol, 2-piperazin-1-yl-ethanol, 8-methyl-1,3-dioxo-2,3,5,8-tetrahydro-1H-[1,2,4]triazolo[1,2-a]pyridazine-5-carboxylic acid ethyl ester, acetamide, amino-acetic acid, carbamic acid methyl ester, carbamic acid tert-butyl ester, carbamoylmethyl-carbamic acid tert-butyl ester, carbonic acid mono-[2-(2-methoxy-ethoxy)-ethyl]ester, ethyl-carbamic acid tert-butyl ester, isobutyric acid 1-hydroxy-ethyl ester, methoxy-benzene, methyl-urea, phenol, phenyl-methanol, phosphoric acid di-tent-butyl ester, phosphoric acid mono-tent-butyl ester, (2-carbamoyl-ethyl)-carbamic acid tert-butyl ester, 3-amino-propionamide, acetic acid 3-acetoxy-2-hydroxy-propyl ester, or urea; optionally substituted with one or more halo, N₃, OH, thiol, nitro, CN, C₁₋₆alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle.

18. The method of claim 15, wherein L is a direct bond and R⁸ chosen from N⁴-(4-methoxy-phenyl)-N⁴-methyl-N²-(3-methylamino-propyl)-quinazoline-2,4-diamine, (1R,2S)-2-amino-1-phenyl-propan-1-ol, (S)-2-amino-2-phenyl-ethanol, (S)-2-amino-3-(4-hydroxy-phenyl)-propionic acid methyl ester, (S)-2-amino-3-phenyl-propan-1-ol, (S)-2-amino-propionic acid, (S)-2-amino-propionic acid tert-butyl ester, 2-amino-3-methyl-butyric acid ethyl ester, N-(2-acetylamino-ethyl)-acetamide, 1-amino-3-{4-[(4-methoxy-phenyl)-methyl-amino]-quinazolin-2-ylamino}-propan-2-ol, 1,3-diamino-propan-2-ol, or piperazine; optionally substituted with one or more halo, N₃, OH, thiol, nitro, CN, C₁₋₆alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle.

19. The method of claim 15, wherein L is —NHCH₂CH₂—, and R⁸ is chosen from 2-(2-amino-ethoxy)-ethanol, N¹-(2-Amino-ethyl)-ethane-1,2-diamine, 8-methyl-1,3-dioxo-2,3,5,8-tetrahydro-1-[1,2,4]triazolo[1,2-a]pyridazine-5-carboxylic acid ethyl ester, carbonic acid mono-[2-(2-methoxy-ethoxy)-ethyl]ester, phosphoric acid di-tent-butyl ester, (S)-2,6-diamino-hexanoic acid, phosphoric acid mono-tent-butyl ester, amino-acetic acid, carbamic acid tert-butyl ester, (R)-2,6-diamino-hexanoic acid, carbamic acid benzyl ester, carbamic acid methyl ester, (S)-2-amino-4-methylsulfanyl-butyric acid, (S)-2-benzyloxycarbonylamino-3-(1H-indol-3-yl)-propionic acid, carbamoylmethyl-carbamic acid tert-butyl ester, (S)-2-amino-succinamic acid, (R)-2-amino-3-(1H-indol-3-yl)-propionic acid, methyl-urea, 2-amino-acetamide, acetamide, and urea.

20. A process for the manufacture of a compound according to Formula I: 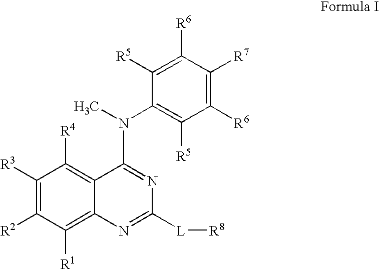
or a pharmaceutically acceptable salt thereof, wherein,
R¹, R², R³, R⁴, R⁵, and R⁶ are each independently chosen from the group consisting of H, C₁₋₃alkyl, C₁₋₃alkoxy, and halo;
R⁷ is C₁₋₃alkoxy; and
L is chosen from —OCH₂CH₂—, —NHCH₂CH₂—, —CH₂NHC(═O)—, —CH₂NHS(═O)₂—, or —CH₂OS(═O)₂—; each optionally substituted with one or more H or C₁₋₃alkyl; with R⁸ chosen from H, OH, amino, 2,6-diamino-hexanoic acid, 2-amino-3-(1H-indol-3-yl)-propionic acid, 2,6-diamino-hexanoic acid, 2-amino-4-methylsulfanyl-butyric acid, 2-amino-succinamic acid, 2-benzyloxycarbonylamino-3-(1H-indol-3-yl)-propionic acid, 2-(2-amino-ethoxy)-ethanol, 2-(2-ethoxy-ethoxy)-ethanol, 2-(2-methoxy-ethoxy)-ethanol, 2-amino-acetamide, 2-methoxy-ethanol, 2-piperazin-1-yl-ethanol, 8-methyl-1,3-dioxo-2,3,5,8-tetrahydro-1H-[1,2,4]triazolo[1,2-a]pyridazine-5-carboxylic acid ethyl ester, acetamide, amino-acetic acid, carbamic acid methyl ester, carbamic acid tert-butyl ester, carbamoylmethyl-carbamic acid tert-butyl ester, carbonic acid mono-[2-(2-methoxy-ethoxy)-ethyl]ester, ethyl-carbamic acid tert-butyl ester, isobutyric acid 1-hydroxy-ethyl ester, methoxy-benzene, methyl-urea, N¹-(2-amino-ethyl)-ethane-1,2-diamine, phenol, phenyl-methanol, phosphoric acid di-tent-butyl ester, phosphoric acid mono-tent-butyl ester, (2-carbamoyl-ethyl)-carbamic acid tert-butyl ester, 3-amino-propionamide, acetic acid 3-acetoxy-2-hydroxy-propyl ester, or urea; each optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH(R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle; or
L is a direct bond; with R⁸ chosen from 3-({4-[(4-methoxy-phenyl)-methyl-amino]-quinazolin-2-ylmethyl}-methyl-amino)-propan-1-ol, N⁴-(4-methoxy-phenyl)-N⁴-methyl-N²-(3-methylamino-propyl)-quinazoline-2,4-diamine, 2-amino-1-phenyl-propan-1-ol, 2-amino-2-phenyl-ethanol, 2-Amino-3-(4-hydroxy-phenyl)-propionic acid methyl ester, 2-amino-3-phenyl-propan-1-ol, 2-amino-propionic acid, 2-amino-propionic acid tert-butyl ester, 2-amino-3-methyl-butyric acid ethyl ester, N-(2-acetylamino-ethyl)-acetamide, 1-amino-3-{4-[(4-methoxy-phenyl)-methyl-amino]-quinazolin-2-ylamino}-propan-2-ol, 1,3-diamino-propan-2-ol, or piperazine; optionally substituted with one or more halo, N₃, OH, thiol, nitro, CN, C₁₋₆ alkyl, C₂₋₆alkenyl, C₂₋₆alkynyl, C₁₋₆alkoxy, C₁₋₆alkylthiol, C₂₋₆alkenyl-O—, C₂₋₆alkynyl-O—, hydroxy-C₁₋₆alkyl, C₁₋₆alkoxy-C₁₋₆alkyl, C₁₋₆acyl, C₁₋₆acyloxy, —C₁₋₆alkyl-C(═O)O—C₁₋₆alkyl, —C(═O)O—C₁₋₆alkyl, C₁₋₆alkyl-C(═O)O—C₁₋₆alkyl-, C₁₋₆acylamido, —N(R^{a})(R^{b}), —C₁₋₆alkyl-C(═O)N(R^{a})(R^{b}), —C(═O)N(R^{a})(R^{b}), N(R^{a})(R^{b})—C₁₋₆alkyl-, wherein R^{a} and R^{b} are independently H, OH (R^{a} and R^{b} are not both OH), C₂₋₆hydroxyalkyl, or C₁₋₆alkyl or R^{a} and R^{b} together with the nitrogen atom to which they are both linked form a 3, 4, 5 or 6-membered heterocycle,
said method comprising reacting a first compound of the following formula: 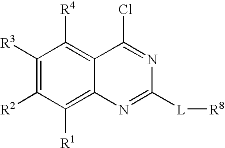
wherein R¹, R², R³, R⁴, L, and R⁸ are as defined above;
with a second compound of the following formula: 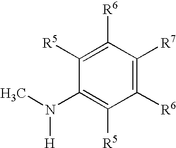
wherein R⁵, R⁶, and R⁷ are as defined above;
to form a product according to Formula I.
